(19) United States
(12) Patent Application Publication
(10) Pub. No.: US 2025/0250574 A1
(43) Pub. Date: Aug. 7, 2025

(54) SIRNA OR SALT THEREOF AND MEDICAMENT FOR INHIBITING EXPRESSION OF TMPRSS6 GENE, AND USE THEREOF

(71) Applicant: BeBetter Med Inc., Guangdong (CN)

(72) Inventors: Xinjian LIU, Guangdong (CN); Fushun FAN, Guangdong (CN); Changgeng QIAN, Guangdong (CN)

(73) Assignee: BeBetter Med Inc., Guangdong (CN)

(21) Appl. No.: 19/093,270

(22) Filed: Mar. 28, 2025

Related U.S. Application Data
(63) Continuation of application No. PCT/CN2023/095104, filed on May 18, 2023.

(30) Foreign Application Priority Data
Sep. 29, 2022 (CN) 202211203337.3
Jan. 19, 2023 (CN) 202310088165.8

(51) Int. Cl. C12N 15/113; A61P 3/00

(52) U.S. Cl. CPC C12N 15/1137; A61P 3/00; C12N 2310/11; C12N 2310/14; C12N 2310/312; C12N 2310/315; C12N 2310/321; C12N 2310/322; C12N 2310/351; C12Y 304/21109

(57) ABSTRACT

The present invention provides an siRNA or a pharmaceutically acceptable salt thereof for inhibiting TMPRSS6 expression in human cells, as well as appropriate modifications of the siRNA to enhance target silencing efficiency and minimize off-target activity. The invention further provides biological agent or pharmaceutical compositions containing the foregoing siRNA for inhibiting TMPRSS6 expression. Through a series of in vitro and in vivo experiments, the present invention has identified siRNA sequences with potent biological activity in suppressing TMPRSS6 expression, demonstrating higher efficacy than TMPRSS6-HCM-9, which is currently the most advanced siRNA in clinical development. These findings support the potential clinical application of the invention in treating disorders associated with dysregulated TMPRSS6 expression or diseases related to iron excess or iron overload, demonstrating significant therapeutic potential.

### CROSS-REFERENCE TO RELATED APPLICATION

This application is a continuation of international application of PCT application serial no. PCT/CN2023/095104, filed on May 18, 2023, which claims the priority benefit of China application no. 202211203337.3, filed on Sep. 29, 2022 and China application no. 202310088165.8, filed on Jan. 19, 2023. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Mar. 21, 2025, is named 136832US-sequencing_listing and is 167,072 bytes in size.

### BACKGROUND

### Technical Field

The present disclosure related to the field of biomedical sciences, and in particular to an siRNA or a pharmaceutically acceptable salt thereof for inhibiting the expression of the TMPRSS6 gene, medicaments, and applications thereof.

### Description of Related Art

RNA interference (RNAi) is a process in which messenger RNA (mRNA) is cleaved by small interfering RNA (siRNA), and is widely used for studying the regulation of gene expression in various cells. Delivery of siRNA into cells in vitro can significantly inhibit gene expression (Jackson et al., 2006, RNA 12:1179-1187). In recent years, RNA interference (RNAi) has become one of the most widely used gene knockout technologies (Hu et al., 2020, Signal Transduct Target Ther 5:101). Small interfering RNA (siRNA) or short single-stranded RNA such as microRNA (miRNA) forms complexes with the RNA-induced silencing complex (RISC), and subsequently binds to complementary mRNA, which is then cleaved with the help of ribonucleases to inhibit the expression of specific genes. The mechanism of RNAi can protect cells from viral infections and transposable elements (Weingartner et al., 2020, Mol Ther Nucleic Acids 21:242-250).

Beta-thalassemia is one of the most common genetic diseases, typically caused by mutations in the beta-globin gene or its promoter, leading to reduced or absent synthesis of beta-globin (Cao and Kan, 2013, Cold Spring Harb Perspect Med 3: a011775; Rivella, 2015, Haematologica 100:418-430). More than 180 different beta-globin gene mutations have been identified in beta-thalassemia patients, which may affect gene function at any level of transcription, processing of primary messenger RNA transcripts, translation, or post-translational stability of the gene product. Beta-thalassemia typically manifests as ineffective erythropoiesis accompanied by extramedullary hematopoiesis, splenomegaly, and systemic iron overload. Affected individuals exhibit extreme clinical heterogeneity, ranging from nearly asymptomatic to life-threatening severe anemia. Beta-thalassemia can be divided into beta-thalassemia major and relatively less severe anemia (non-transfusion-dependent thalassemia or thalassemia intermedia) (Musallam et al., 2021, Am J Hematol 96:1518-1531).

In thalassemia patients, the main regulator of iron homeostasis, hepcidin, is chronically suppressed. This suppression leads to uncontrolled dietary iron absorption and insufficient iron retention in the reticuloendothelial system. Patients absorb abnormally high levels of iron, exacerbating ineffective erythropoiesis and anemia, which necessitates iron chelation to prevent clinical sequelae associated with iron overload (Richard et al., 2020, Am J Hematol 95:68-77). Regulation of iron homeostasis in patients is necessary to prevent toxicity due to iron excess (Nualkaew et al., 2021, Mol Ther 29:2841-2853). If left untreated, iron overload can lead to organ failure and death. Consequently, management of iron overload in β-thalassemia and other iron-related diseases has become a major focus (Beliveau et al., 2019, Cell Chem Biol 26:1559-1572). The production of hepcidin is primarily regulated by a transmembrane serine protease encoded by the TMPRSS6 gene. TMPRSS6 (transmembrane protease, serine 6), also known as matriptase-2, is a type II serine protease. It is predominantly expressed in the liver, but high levels of TMPRSS6 mRNA have also been detected in the kidney. TMPRSS6 plays a role in iron homeostasis by inhibiting the activation of hepcidin through cleavage of hemojuvelin, and inhibition of TMPRSS6 expression can regulate iron homeostasis, reduce iron overload, and thus alleviate symptoms of thalassemia (Ginzburg and Rivella, 2011, Blood 118:4321-4330).

TMPRSS6-HCM-9 is an siRNA targeting TMPRSS6, which is currently undergoing a Phase 1 clinical trials for the treatment of thalassemia. Previous phase 1 clinical trials of SLN124 in healthy volunteers have demonstrated that SLN124 effectively reduced plasma iron levels, exhibited a favorable safety profile, and had a prolonged duration of action. The sequence of SLN124 is disclosed in patent US_11174483_B2. Currently, there are limited therapeutic options available for the treatment of thalassemia, and an siRNA-based therapy has good market prospects.

Ineffective erythropoiesis and chronic anemia result in iron metabolism disorders and systemic iron overload, which in turn leads to cardiac, hepatic, and endocrine dysfunction. Treatment options for thalassemia remain limited, with red blood cell transfusion being the primary therapeutic approach (Longo et al.,2021, Int J Mol Sci 22). There is an urgent need for novel methods to effectively treat disorders associated with iron overload.

Polycythemia is classified into secondary polycythemia and polycythemia vera. Secondary polycythemia can be alleviated by addressing the underlying cause. Polycythemia vera (PV) is a myeloproliferative disorder (MPDS) that often leads to abnormally high hematocrit (Hct) and hemoglobin (Hb) concentrations, which can result in thromboembolic complications. Loss of function of the TMPRSS6 gene leads to increased production of hepcidin, thereby improving abnormal erythropoiesis and preventing or limiting iron overload. Targeting TMPRSS6 with oligonucleotides provides an approach for preventing or treating polycythemia. Currently, an antisense oligonucleotide drug targeting TMPRSS6, IONIS-TMPRSS6-LRx (ISIS 702843), is in phase 2 clinical trials for the treatment of polycythemia vera. Developing additional siRNA-based therapies will contribute to treating these disorders and provide more clinical options.

### SUMMARY

Based on this, the objective of the present invention provides a double-stranded siRNA or a pharmaceutically acceptable salt thereof for inhibiting TMPRSS6 expression, as well as its applications, which exhibits robust in vitro and in vivo efficacy with reduced off-target activity.

The first aspect of the present invention provides an siRNA or a pharmaceutically acceptable salt thereof for inhibiting TMPRSS6 expression. The siRNA comprises a sense strand and an antisense strand, which form a double-stranded structure through base pairing. The nucleotide sequence is selected from any one of the sequences (a) to (e) as set forth herein, or a sequence differing by one, two, or three nucleotides from any one of the sequences (a) to (e):
(a) The double-stranded siRNA designated BBD-2051, comprising a sense strand having the nucleotide sequence as set forth in SEQ ID NO: 103 (UGGGAACUUACUACAACUCCA), and an antisense strand having the nucleotide sequence as set forth in SEQ ID NO:104 (UGGAGUUGUAGUAAGUUCCCAGG).
(b) The double-stranded siRNA designated BBD-2083, comprising a sense strand having the nucleotide sequence as set forth in SEQ ID NO:167 (UGCUACUCUGGUAUUUCCUAA), and an antisense strand having the nucleotide sequence as set forth in SEQ ID NO:168 (UUAGGAAAUACCAGAGUAGCACC).
(c) The double-stranded siRNA designated BBD-2047, comprising a sense strand having the nucleotide sequence as set forth in SEQ ID NO:95 (CGCCUGGGAACUUACUACAAU), and an antisense strand having the nucleotide sequence as set forth in SEQ ID NO:96 (AUUGUAGUAAGUUCCCAGGCGGG).
(d) The double-stranded siRNA designated BBD-2086, comprising a sense strand having the nucleotide sequence as set forth in SEQ ID NO:173 (GGGUGCUACUCUGGUAUUUCA), and an antisense strand having the nucleotide sequence as set forth in SEQ ID NO:174 (UGAAAUACCAGAGUAGCACCCCC).
(e) The double-stranded siRNA designated BBD-2087, comprising a sense strand having the nucleotide sequence as set forth in SEQ ID NO:175 (GGGGUGCUACUCUGGUAUUUA), and an antisense strand having the nucleotide sequence as set forth in SEQ ID NO:176 (UAAAUACCAGAGUAGCACCCCCG).

In certain embodiments, the sense strand comprises no more than 3, 2, 1, or 0 unmodified nucleotides, and the modified nucleotides in the sense strand are selected from 2′-O-methyl-modified nucleotides, 2′-deoxynucleotides, and 2′-fluoro-modified nucleotides. The sense strand comprises 1 to 3 phosphorothioate linkages at the 5′-terminus.

In certain embodiments, the antisense strand comprises no more than 3, 2, 1, or 0 unmodified nucleotides. The modified nucleotides in the antisense strand are selected from 2′-O-methyl-modified nucleotides, 2′-deoxynucleotides, 2′-fluoro-modified nucleotides, 5′-(E)-vinylphosphonate nucleotides, and isomer of glycerol nucleic acid (isoGNA). The antisense strand comprises 1 to 3 phosphorothioate linkages at both the 5′-terminus and the 3′-terminus. The double-stranded RNAi agent comprises at least one strand with a 3′-overhang of 1 to 3 nucleotides.

In some preferred embodiments, the isomer of glycerol nucleic acid (isoGNA) nucleotide is located in at least one position between position 4 and 8 counted from the 5′-terminus of the antisense strand, so as to reduce off-target activity of an siRNA or a pharmaceutically acceptable salt thereof and enhance safety.

In certain embodiments, antisense strand of the complementary siRNA at positions 11 to 13 of the 5′-terminus and/or the sense strand of the complementary siRNA at positions 9 to 11 of the 5′-terminus together comprise a total of 1 to 6 2′-deoxynucleotides.

Wherein, the isomer of glycerol nucleic acid (isoGNA) nucleotide is selected from at least one of the following structures:

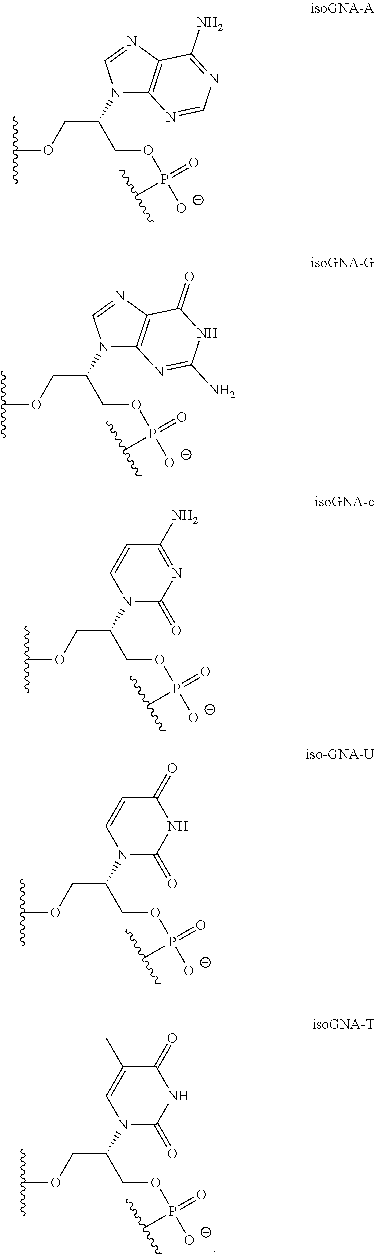

Wherein, the 2′-deoxy modified nucleotides is selected from at least one of the following structures:

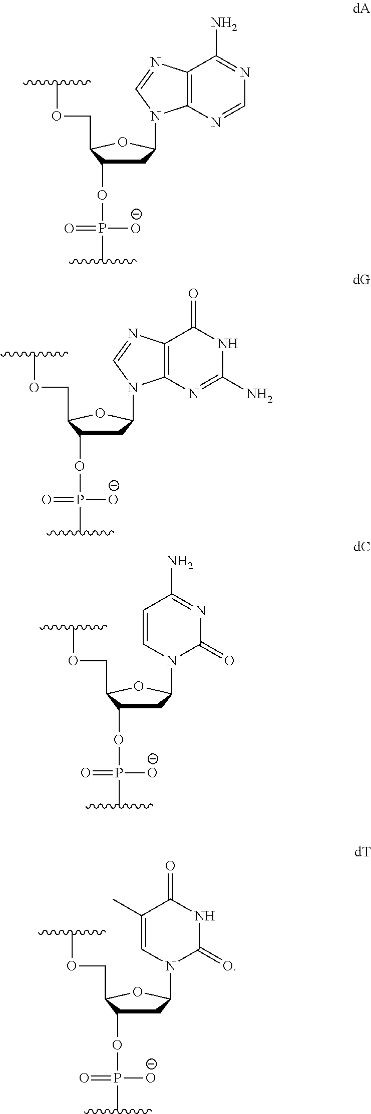

Wherein, the structure of the uridine-5′-(E)-vinylphosphonate nucleotide is as follows:

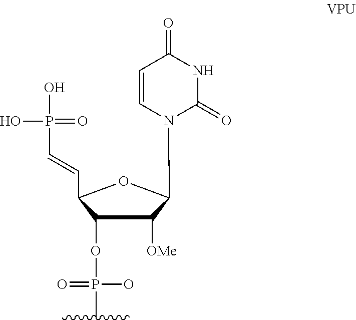

In some preferred embodiments, the modified duplex ID is selected from the group consisting BBD-2051.210, BBD-2051.211, BBD-2051.28, BBD-2083.313, BBD-2083.413, BBD-2051.25, BBD-2051.26, BBD-2051.27, BBD-2051.28, BBD-2051.29, BBD-2051.213, BBD-2051.214, BBD-2051.215, BBD-2051.217, BBD-2051.218, and BBD-2051.219; BBD-2083.41, BBD-2083.410, BBD-2083.411, BBD-2083.412, BBD-2083.414, BBD-2083.415, BBD-2083.416, BBD-2083.418, BBD-2083.419, BBD-2083.420, BBD-2083.422, BBD-2083.423, and BBD-2083.424.

In some more preferred embodiments, the modified siRNA is selected from the group consisting:
BBD-2051.210:
sense strand: mU*mG*mGmGmAmAfCmUfUfAfCmUmAmCmAmAmCmUmCmCmA,
Antisense strand: VPU*fG*mGmAmGisoGNA-TmUmGmUfAmGdTmAfAmGfUmUmCmCmCmA*mG*mG.
BBD-2051.211:
sense strand: mU*mG*mGmGmAmAfCmUfUfAfCmUmAmCmAmAmCmUmCmCmA,
Antisense strand: VPU*fG*mGmAmGmUisoGNA-TmGmUfAmGdTmAfAmGfUmUmCmCmCmA*mG*mG
BBD-2051.28:
sense strand: mU*mG*mGmGmAmAfCmUfUfAfCmUmAmCmAmAmCmUmCmCmA,
Antisense strand: VPU*fG*mGmAmGmUmUmGmUfAmGdTmAfAmGfUmUmCmCmCmA*mG*mG
BBD-2083.313:
sense strand: mU*mG*mCmUmAmCfUmCfUfGfGmUmAmUmUmUmCmCmUmAmA,
Antisense strand: VPU*fU*mAmGmGmAmAmAmUfAmCdCmAfGmAfGmUmAmGmCmA*mC*mC>
BBD-2083.413:
sense strand: mU*mG*mCmUmAmCfUmCfUfGdGmUmAmUmUmUmCmCmUmAmA,
Antisense strand: VPU*fU*mAmGmGmAmAmAmUfAmCdCmAfGmAfGmUmAmGmCmA*mC*mC;

The second aspect of the present invention provides a pharmaceutical composition for inhibiting TMPRSS6 expression, comprising any one of the siRNAs or a pharmaceutically acceptable salt thereof as described above, and a targeting delivery ligand or delivery vehicle conjugated to or encapsulating the siRNA.

In certain embodiments, the ligand is an N-acetylgalactosamine (GalNAc) derivative.

In certain embodiments, the structure of the ligand is as follows:

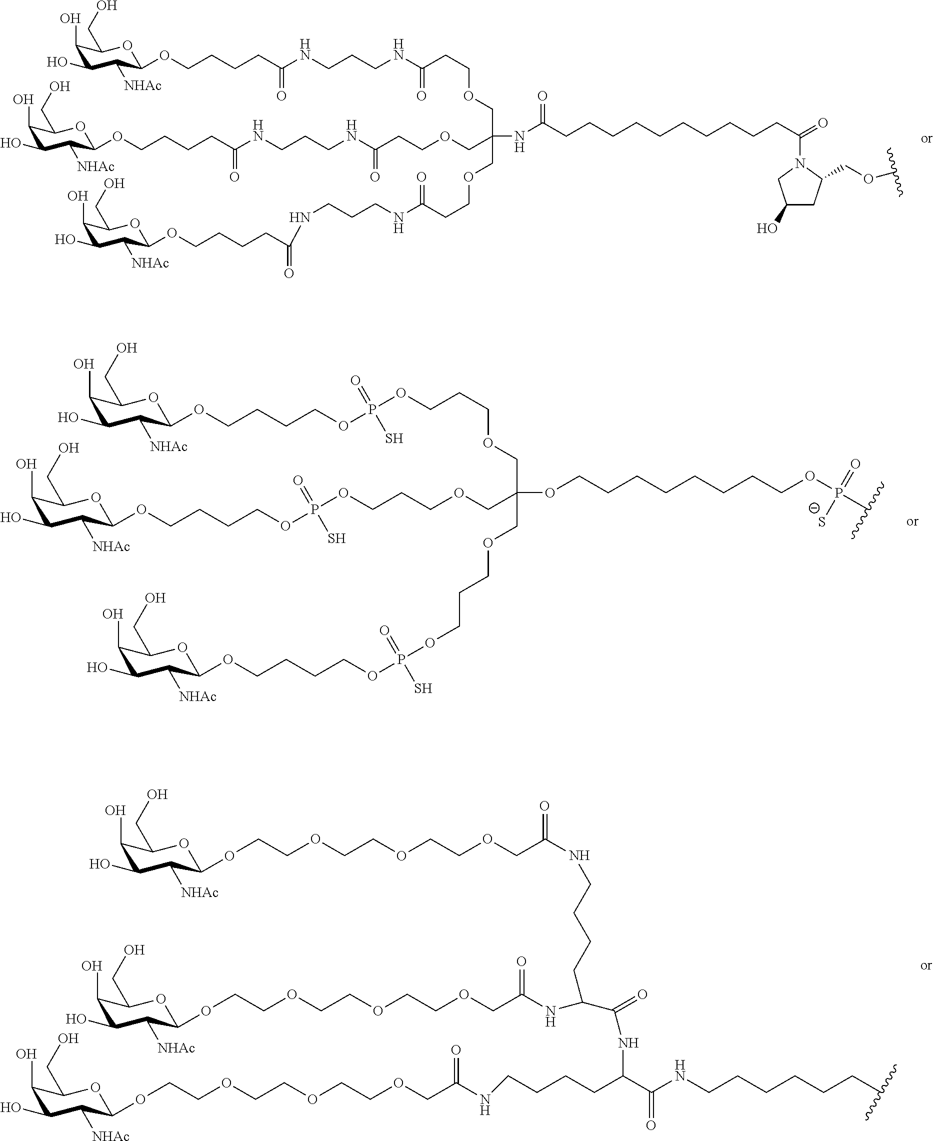

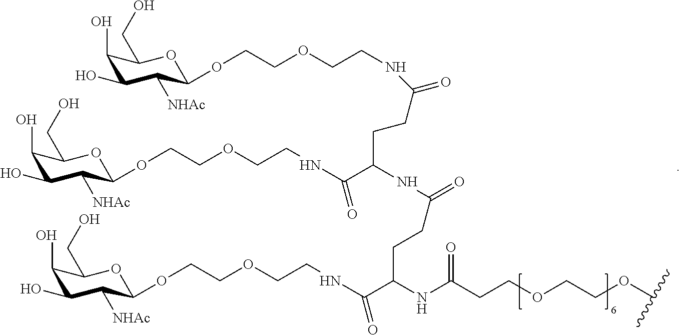

In certain embodiments, the ligand is conjugated to the 3′-terminus of the sense strand of the siRNA.

In certain embodiments, the ligand is conjugated to the 3′-terminus of the sense strand of the siRNA, as illustrated in the following schematic:

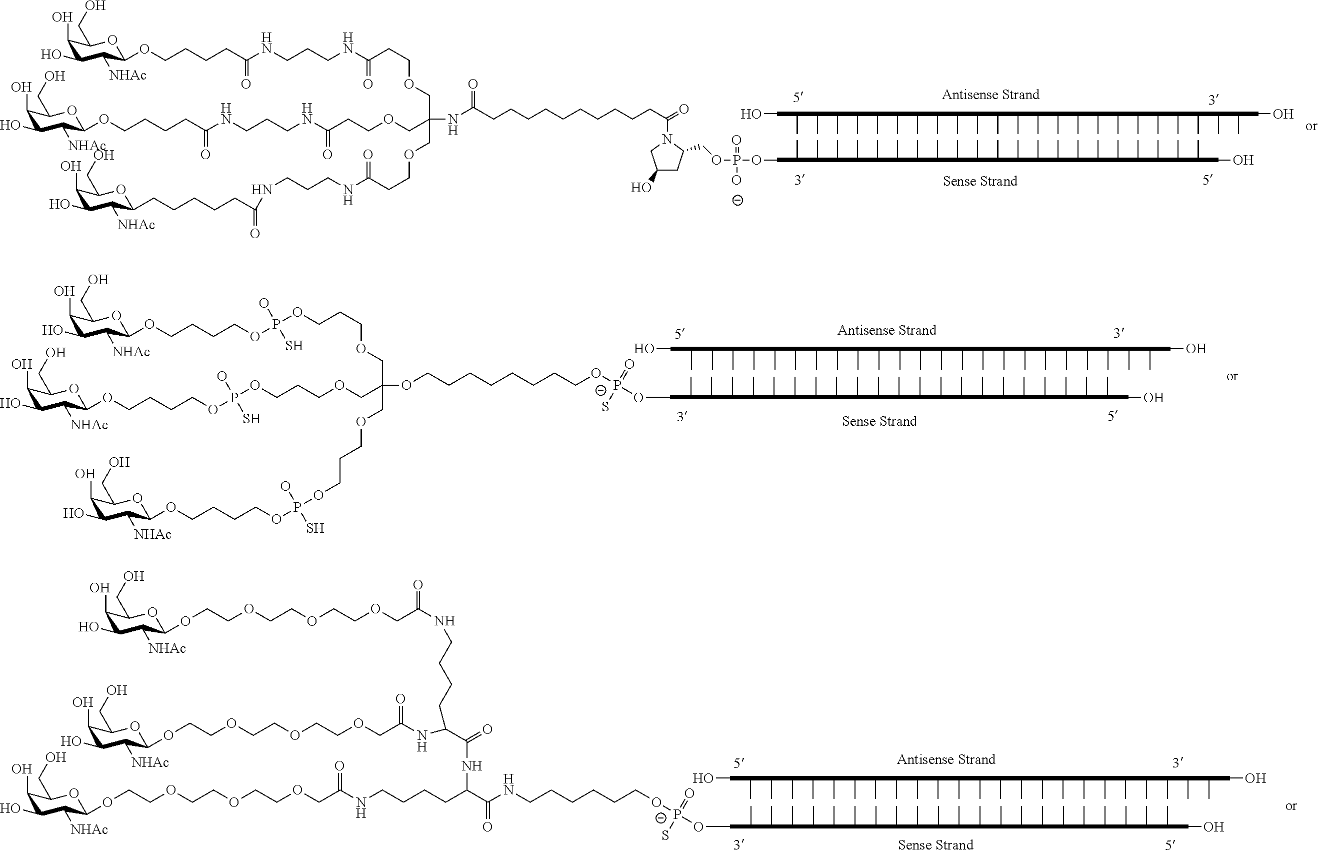

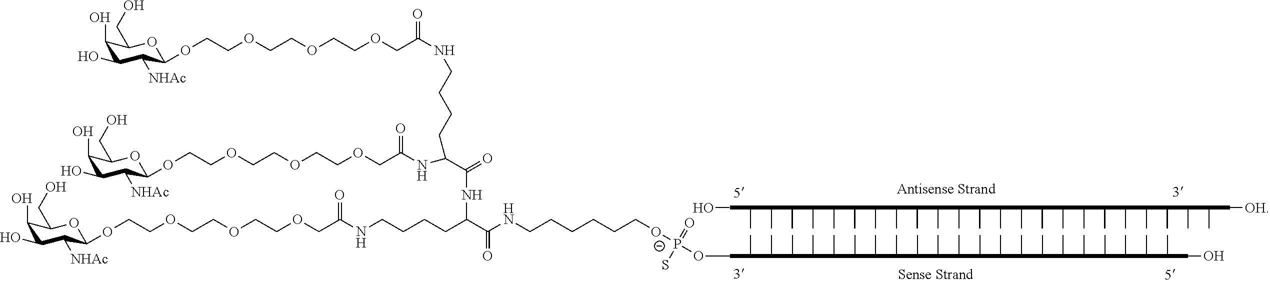

The third aspect of the present invention provides the use of the above-mentioned siRNA or the pharmaceutically acceptable salt thereof in the manufacture of a biological agent or pharmaceutical composition for inhibiting TMPRSS6 expression.

The fourth aspect of the present invention provides the use of the biological agent or the pharmaceutical composition in the manufacture of a medicament for preventing and treating a disorder associated with TMPRSS6 or a disorder associated with iron excess or iron overload.

The fifth aspect of the present invention provides a method of inhibiting TMPRSS6 expression in a cell, the method comprising:
(a) contacting the cell with any one of the foregoing siRNA or a pharmaceutically acceptable salt thereof or any of the foregoing biological agents or pharmaceutical compositions; and (b) maintaining the cells produced in step (a) for a time sufficient to obtain degradation of the mRNA transcript of a TMPRSS6 gene, thereby inhibiting expression of the TMPRSS6 gene in the cell

The sixth aspect of the present invention provides a method of treating a subject suffering from a TMPRSS6-related disorder or a disease associated with iron excess or iron overload, comprising administering to the subject a therapeutically effective amount of any one of the foregoing siRNAs or pharmaceutically acceptable salts thereof, or any one of the foregoing biological agents or pharmaceutical compositions, thereby treating the subject.

In certain embodiments, the cell is within the subject.

In certain embodiments, the subject is a human.

In certain embodiments, the TMPRSS6 expression is inhibited by at least about 30%, about 40%, about 50%, about 60%, about 70%, about 80%, about 90%, about 95%, about 98%, or about 100%.

In certain embodiments, the serum hepcidin concentration of the subject is increased by at least about 10%; and/or the serum iron concentration of the subject is decreased by at least about 20%; and/or a percent transferrin saturation of the subject is decreased by at least about 20%.

In certain embodiments, a disorder associated with TMPRSS6 or a disease associated with iron excess or iron overload is selected from polycythemia, thalassemia, hemochromatosis, myelodysplastic syndrome, porphyria cutanea tarda, aplastic anemia, sideroblastic anemia, iron-refractory iron deficiency anemia, hereditary anemia, severe chronic hemolytic anemia, Parkinson's disease, Alzheimer's disease, Friedreich's ataxia, or microcytic anemia, transfusion-induced iron overload, and iron overload associated with non-alcoholic fatty liver disease (NAFLD).

In certain embodiments, the thalassemia is selected from α-thalassemia, β-thalassemia, and 8-thalassemia.

In certain embodiments, the polycythemia is polycythemia vera.

In certain embodiments, the hereditary anemia is selected from sickle-cell anemia, thalassemia, Fanconi anemia, Diamond-Blackfan anemia, Shwachman-Diamond syndrome, red blood cell membrane disorders, glucose-6-phosphate dehydrogenase deficiency, and hereditary hemorrhagic telangiectasia.

The inventors of the present application have conducted research on siRNA sequences and, through extensive experiments, identified multiple siRNA sequences capable of inhibiting TMPRSS6 expression. Based on these findings, appropriate modifications were further made to enhance target silencing efficiency and reduce off-target activity. Through a series of in vitro and in vivo experiments, siRNA sequences with robust biological activity in inhibiting TMPRSS6 expression were obtained, demonstrating superior activity compared to the most advanced clinical candidate, TMPRSS6-HCM-9. These siRNA sequences are expected to be applied in the clinical treatment of TMPRSS6-related disorders or diseases associated with iron overload or iron excess.

### BRIEF DESCRIPTION OF THE DRAWINGS

FIG. 1 illustrates the evaluation of the effects of different modified BBD-2047 sequences, BBD-2051 sequences, BBD-2083 sequences, and TMPRSS6-HCM-9 sequence on hepatic human TMPRSS6 expression in mice expressing human TMPRSS6 (1 mg/kg).

FIG. 2 illustrates the evaluation of the effects of different modified BBD-2083 sequences and TMPRSS6-HCM-9 sequence on hepatic hTMPRSS6 expression in mice expressing human TMPRSS6 (3 mg/kg).

FIG. 3 illustrates the evaluation of the effects of different modified BBD-2051 sequences and TMPRSS6-HCM-9 sequence on hepatic hTMPRSS6 expression in mice expressing human TMPRSS6 (3 mg/kg).

FIG. 4 illustrates the evaluation of the effects of different modified BBD-2051 sequences on hepatic mTMPRSS6 expression in wild-type mice (10 mg/kg).

FIG. 5 illustrates the evaluation of the effects of different modified BBD-2051 sequences on serum iron concentration in wild-type mice (10 mg/kg).

FIG. 6 illustrates the evaluation of the off-target activity of different modified BBD-2051 sequences.

FIG. 7 illustrates the evaluation of the off-target activity of different modified BBD-2083 sequences and AD-1554911.

### DESCRIPTION OF THE EMBODIMENTS

To facilitate understanding of the present invention, a more detailed description is provided below. The invention can be implemented in numerous forms and is not limited to the embodiments described herein. Rather, these embodiments are provided to enable a thorough and comprehensive understanding of the disclosed content of the invention.

In the following embodiments, experimental methods, unless otherwise specified, are typically conducted under conventional conditions, such as those described in Sambrook et al., Molecular Cloning: A Laboratory Manual (New York: Cold Spring Harbor Laboratory Press, 1989), or according to the conditions recommended by the manufacturer. Various commonly used chemical reagents employed in the embodiments are commercially available products.

Unless otherwise defined, all technical and scientific terms used herein have the same meanings as commonly understood by one of ordinary skill in the art to which the present invention belongs. The terminology used in the specification is provided solely for the purpose of describing specific embodiments only and is not intended to limit the invention. The term ‘and/or’ as used in the present invention encompasses any and all combinations of the listed items.

In order that the present invention may be more readily understood, certain terms are defined.

Iron overload refers to the excessive accumulation of iron in the body, resulting in structural damage and functional impairment of vital organs, particularly the heart, liver, pituitary gland, pancreas, and joints.

Polycythemia refers to an elevated erythrocyte count and hemoglobin concentration in a given volume of blood, exceeding the upper limit of the reference range.

Hemochromatosis is a disorder of iron metabolism characterized by excessive iron accumulation in the body, resulting from a high-iron diet, frequent blood transfusions, or systemic diseases.

Thalassemia, also known as “marine anemia”, is officially termed “globin synthesis disorder anemia” as designated by China National Committee for Terminology in Sciences and Technologies. This disease is characterized by the impairment or complete inhibition of one or more globin chain syntheses, leading to abnormal hemoglobin (Hb) composition and subsequent chronic hemolytic anemia.

The expression of the TMPRSS6 gene can be assessed based on the level of any variable associated with TMPRSS6 gene expression, e.g., TMPRSS6 mRNA level in tissues or serum, TMPRSS6 protein level, hepcidin mRNA level, hepcidin protein level, transferrin saturation level, or iron level. Inhibition can be assessed by a decrease in an absolute or relative level of one or more of these variables compared with a control level. The control level may be any type of control level that used in the art, e.g., pre-dose baseline level, or a level measured from a similar subject, cell, or sample that is untreated or treated with a control.

In certain embodiments, the diseases related to TMPRSS6 expression or associated with iron excess or iron overload are selected from polycythemia, thalassemia, hemochromatosis, myelodysplastic syndrome, porphyria cutanea tarda, aplastic anemia, sideroblastic anemia, iron-refractory iron deficiency anemia, hereditary anemia, severe chronic hemolysis, Parkinson's disease, Alzheimer's disease, Friedreich's ataxia, or microcytic anemia, transfusion-dependent iron overload, and NAFLD-associated iron overload.

The siRNA agent of the present invention can be delivered to cells, such as those within a subject (e.g., a human subject, such as one suffering from a TMPRSS6-related disorder such as hemochromatosis), via various routes. For instance, delivery can be accomplished by bringing the cells into contact with the siRNA of the present invention either ex vivo or in vivo. In vivo delivery can also be achieved by administering to the subject a composition comprising the siRNA or its salt.

Generally, any method of delivering a nucleic acid molecule (either ex vivo or in vivo) can be adapted for use with existing delivery technologies. For in vivo delivery, factors to consider in order to deliver an siRNA molecule include, for example, biological stability of the delivered molecule, prevention of non-specific effects, and accumulation of the delivered molecule in the target tissue. The non-specific effects of an iRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered.

For systemic iRNA administration to treat diseases, the RNA can be modified or delivered via a drug delivery system; both methods act to prevent the rapid degradation of dsRNA by endonucleases and exonucleases in vivo. Modifications to RNA or pharmaceutical carriers can also enable the siRNA composition to target the target specific tissues and avoid off-target effects. siRNA molecules can be chemically conjugated to lipophilic moieties such as cholesterol, for example, lipid particles, to enhance cellular uptake and prevent degradation.

The present invention also includes pharmaceutical compositions and formulations

which include the iRNAs of the invention. In one embodiment, provided herein are

pharmaceutical compositions containing an iRNA, as described herein, and a pharmaceutically acceptable carrier. The pharmaceutical compositions containing the iRNA are useful for treating a TMPRSS6 associated disease or disorder, e.g. hemochromatosis. Such pharmaceutical compositions are formulated based on the mode of delivery.

In certain embodiments, the compositions are formulated for systemic administration via parenteral routes, e.g., intravenous (IV) delivery or subcutaneous (SC) delivery. In the methods of the present invention, the siRNA or its salt pharmaceutical compositions can be administered in a solution, preferably in a sterile solution, e.g., by injection.

In the present invention, the term “Therapeutically effective amount,” as used herein, is intended to include the amount of an RNAi agent that, when administered to a patient for treating a TMPRSS6 associated disease, is sufficient to effect treatment of the disease (e.g., by diminishing, ameliorating or maintaining the existing disease or one or more symptoms of disease). The “therapeutically effective amount” may vary depending on the RNAi agent, how the agent is administered, the disease and its severity and the history, age, weight, family history, genetic makeup, stage of pathological processes mediated by TMPRSS6 expression, the types of preceding or concomitant treatments, if any, and other individual characteristics of the patient to be treated.

In the present invention, the term “Prophylactically effective amount,” as used herein, is intended to include the amount of an RNAi agent that, when administered to a subject who does not yet experience or display symptoms of a TMPRSS6-associated disease, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the siRNA agent, how the agent is administered, the degree of risk of disease,

and the history, age, weight, family history, genetic makeup, the types of preceding or concomitant treatments, if any, and other individual characteristics of the patient to be treated.

The “therapeutically effective amount” or “prophylactically effective amount” also refer to an amount of an siRNA that produces some desired local or systemic effect at a reasonable benefit/risk ratio applicable to any treatment.

The targeted delivery ligand or other types of delivery vehicles of the present invention, wherein the ligand is preferably being an N-acetylgalactosamine derivative (GalNAc carrier), or other types of delivery vehicles, such as liposomes specifically targeting hepatocytes, may also be used in the present invention, provided that they are capable of delivering siRNA.

In recent years, the GalNAc delivery vehicle has been extensively studied. GalNAc-nucleic acid are conjugates of carbohydrate compounds and nucleic acids, where N-acetylgalactosamine is covalently attached in a trivalent manner to the 3′- and/or 5′-ends of RNA sense strands of different sequences, forming GalNAc-siRNA drugs enabling specific delivery to liver cells, and allowing cellular uptake via endocytosis to deliver the drug and exert its function.

In the following embodiments, the structure of the GalNAc delivery vehicle and its conjugation with siRNA are illustrated in the following schematic:

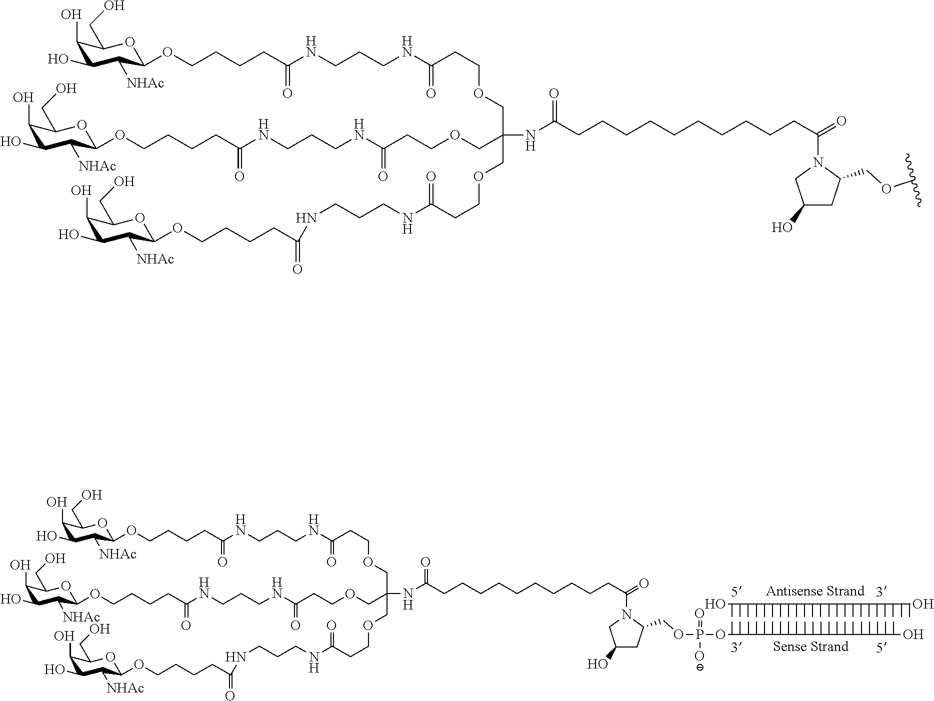

The abbreviations and structures of nucleotide monomers used in the representation of nucleic acid sequences are as follows:

| | |
|---|---|
| A | Adenosine-3′-phosphate |
| T | 5′-methyluridine-3′-phosphate |
| C | cytidine-3′-phosphate |
| G | guanosine-3′-phosphate |
| U | Uridine-3′-phosphate |
| dA | 2′-Deoxyadenosine-3′-phosphate |
| dT | 2′-deoxythymidine-3′-phosphate |
| dC | 2′-Deoxycytidine-3′-phosphate |
| dG | 2′-Deoxyguanosine-3′-phosphate |
| dU | 2′-Deoxyuridine-3′-phosphate |
| mA | 2′-O-Methyladenosine-3′-phosphate |
| mC | 2′-O-Methylcytidine-3′-phosphate |
| mG | 2′-O-Methylguanosine-3′-phosphate |
| mU | 2′-O-Methyluridine-3′-phosphate |
| fA | 2′-Fluoroadenosine-3′-phosphate |
| fC | 2′-Fluorocytidine-3′-phosphate |
| fG | 2′-Fluoroguanosine-3′-phosphate |
| fU | 2′-Fluorouridine-3′-phosphate |
| VPU | Uridine-5′-(E)-vinylphosphonate nucleotide |
| isoGNA-A | Adenosine-isoglycerol nucleotide |
| isoGNA-T | Thymine-isoglycerol nucleotide |
| isoGNA-C | Cytosine-isoglycerol nucleotide |
| isoGNA-G | Guanosine-isoglycerol nucleotide |
| isoGNA-U | Uridine-isoglycerol nucleotide |
| * | Phosphorothioate linkage |

The specific structure is as follows:

[chemical structure]

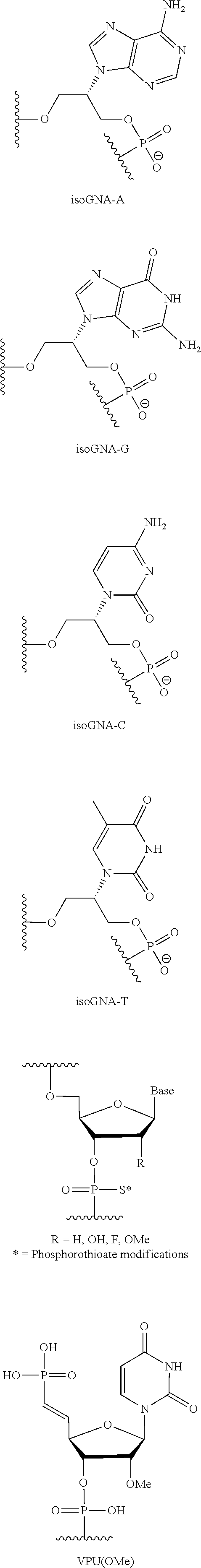

Those skilled in the art will appreciate that the pharmaceutically acceptable salts of the siRNA may include sodium salts or potassium salts; for example, a sodium salt of the siRNA may be generated during the purification process.

The present invention is further described in detail by reference to the following specific embodiments.

### Example 1: Synthesis of siRNA

0.2-1 μmol of oligonucleotides were synthesized using the solid-phase oligonucleotide synthesis protocol on a 12-channel nucleic acid synthesizer of Beijing Qingke Biotechnology Co., Ltd. For siRNA sequences containing GalNAc, synthesis was carried out CPG column preloaded with GalNAc. The synthesized oligonucleotides were treated with an ammonolysis reagent and incubated at 45-80° C. to release them from the solid-phase support. The crude oligonucleotides were then precipitated with ethanol, centrifuged at high speed to remove the supernatant, and the precipitation process was repeated twice. The resulting precipitate was resuspended in DEPC-treated water. The crude oligonucleotides were purified using ion-pair HPLC, and the collected product was lyophilized in a vacuum centrifugal dryer until a powdered form was obtained. The purified product was dissolved in DEPC-treated water and analyzed using TOF LC-MS. The concentration of oligonucleotides was determined, and the volumes of equimolar sense and antisense strands were calculated accordingly. Equimolar amounts of sense and antisense strands were mixed thoroughly and annealed by heating at 95° C. for 5 minutes and then cooling to room temperature naturally to form duplex siRNA.

TABLE 1
Sense and antisense sequences of unmodified siRNA. TMPRSS6-HCM-9 is a
clinical-stage sequence developed by Silence Therapeutics
(Publication No.: CN113164768A) and serves as a positive control sequence.

| | | | | | Antisense | |
|---|---|---|---|---|---|---|
| | Sense | Sense strand | Range | Antisense | strand | Range |
| Duplex | strand | sequence | within | strand | sequence | within |
| ID | sequence ID | 5′-3′ | NM_153609.4 | Sequence ID | 5′-3′ | NM_153609.4 |
| TMPR | S0 (1) | UCACCUGC | 560_578 | AS0 (2) | AACCAGAA | 560_578 |
| SS6- | | UUCUUCUG | | | GAAGCAGG | |
| hcm-9 | | GUU | | | UGA | |
| BBD- | S1(3) | GCCAAAGC | 463_483 | AS01 (4) | UAGCAUCU | 461_483 |
| 2001 | | CCAGAAGA | | | UCUGGGCU | |
| | | UGCUA | | | UUGGCGG | |
| BBD- | S2(5) | UCCUUUGG | 541_561 | AS02(6) | UAGGGGUC | 539_561 |
| 2002 | | GGAGGGAC | | | CCUCCCCA | |
| | | CCCUA | | | AAGGAAU | |
| BBD- | S3(7) | GUGAUCCU | 724_744 | AS03(8) | UACACUGG | 722_744 |
| 2003 | | GGAAGCCA | | | CUUCCAGG | |
| | | GUGUA | | | AUCACUA | |
| BBD- | S4(9) | UGAUCCUG | 725_745 | AS04(10) | UCACACUG | 723_745 |
| 2004 | | GAAGCCAG | | | GCUUCCAG | |
| | | UGUGA | | | GAUCACC | |
| BBD- | S5(11) | GAUCCUGG | 726_746 | AS05(12) | UUCACACU | 724_746 |
| 2005 | | AAGCCAGU | | | GGCUUCCA | |
| | | GUGAA | | | GGAUCAC | |
| BBD- | S6(13) | CCCUCUCU | 1234_1244 | AS06(14) | UAAGCCGU | 1232_1244 |
| 2006 | | GGACUACG | | | AGUCCAGA | |
| | | GCUUA | | | GAGGGCA | |
| BBD- | S7(15) | GAUCCAGA | 1326_1346 | AS07(16) | UACAGCCU | 1324_1346 |
| 2007 | | ACAGGAGG | | | CCUGUUCU | |
| | | CUGUA | | | GGAUCGU | |
| BBD- | S8(17) | AUCCAGAA | 1327_1347 | AS08(18) | ACACAGCC | 1325_1347 |
| 2008 | | CAGGAGGC | | | UCCUGUUC | |
| | | UGUGU | | | UGGAUCG | |
| BBD- | S9(19) | UCCAGAAC | 1328_1348 | AS09(20) | UACACAGC | 1326_1348 |
| 2009 | | AGGAGGCU | | | CUCCUGUU | |
| | | GUGUA | | | CUGGAUC | |
| BBD- | S10(21) | CCAGAACA | 1329_1349 | AS10(22) | UCACACAG | 1327_1349 |
| 2010 | | GGAGGCUG | | | CCUCCUGU | |
| | | UGUGA | | | UCUGGAU | |
| BBD- | S11(23) | CAGAACAG | 1330_1350 | AS11(24) | UCCACACA | 1328_1350 |
| 2011 | | GAGGCUGU | | | GCCUCCUG | |
| | | GUGGA | | | UUCUGGA | |
| BBD- | S12(25) | GAACAGGA | 1332_1352 | AS12(26) | AAGCCACA | 1330_1352 |
| 2012 | | GGCUGUGU | | | CAGCCUCC | |
| | | GGCUU | | | UGUUCUG | |
| BBD- | S13(27) | UCACCAUC | 1406_1426 | AS13(28) | UGGAGGUG | 1404_1426 |
| 2013 | | AACUUCAC | | | AAGUUGAU | |
| | | CUCCA | | | GGUGAUC | |
| BBD- | S14(29) | CACCAUCA | 1407_1427 | AS14(30) | UGGGAGGU | 1405_1427 |
| 2014 | | ACUUCACC | | | GAAGUUGA | |
| | | UCCCA | | | UGGUGAU | |
| BBD- | S15(31) | ACCAUCAA | 1408_1428 | AS15(32) | UUGGGAGG | 1406_1428 |
| 2015 | | CUUCACCU | | | UGAAGUUG | |
| | | CCCAA | | | AUGGUGA | |
| BBD- | S16(33) | CCAUCAAC | 1409_1429 | AS16(34) | UCUGGGAG | 1407_1429 |
| 2016 | | UUCACCUC | | | GUGAAGUU | |
| | | CCAGA | | | GAUGGUG | |
| BBD- | S17(35) | CAUCAACU | 1410_1430 | AS17(36) | AUCUGGGA | 1408_1430 |
| 2017 | | UCACCUCC | | | GGUGAAGU | |
| | | CAGAU | | | UGAUGGU | |
| BBD- | S18(37) | AUCAACUU | 1411 1431 | AS18(38) | UAUCUGGG | 1409_1431 |
| 2018 | | CACCUCCC | | | AGGUGAAG | |
| | | AGAUA | | | UUGAUGG | |
| BBD- | S19(39) | UCAACUUC | 1412_1432 | AS19(40) | AGAUCUGG | 1410_1432 |
| 2019 | | ACCUCCCA | | | GAGGUGAA | |
| | | GAUCU | | | GUUGAUG | |
| BBD- | S20(41) | CAACUUCA | 1413_1433 | AS20(42) | UAGAUCUG | 1411_1433 |
| 2020 | | CCUCCCAG | | | GGAGGUGA | |
| | | AUCUA | | | AGUUGAU | |
| BBD- | S21(43) | AACUUCAC | 1414_1434 | AS21(44) | UGAGAUCU | 1412_1434 |
| 2021 | | CUCCCAGA | | | GGGAGGUG | |
| | | UCUCA | | | AAGUUGA | |
| BBD- | S22(45) | ACUUCACC | 1415_1435 | AS22(46) | UGGAGAUC | 1413_1435 |
| 2022 | | UCCCAGAU | | | UGGGAGGU | |
| | | CUCCA | | | GAAGUUG | |
| BBD- | S23(47) | CUUCACCU | 1416_1436 | AS23(48) | AGGGAGAU | 1414_1436 |
| 2023 | | CCCAGAUC | | | CUGGGAGG | 1436 |
| | | UCCCU | | | UGAAGUU | |
| BBD- | S24(49) | UUCACCUC | 1417_1437 | AS24(50) | UAGGGAGA | 1415_1437 |
| 2024 | | CCAGAUCU | | | UCUGGGAG | |
| | | CCCUA | | | GUGAAGU | |
| BBD- | S25(51) | UCACCUCC | 1418_1438 | AS25(52) | UGAGGGAG | 1416_1438 |
| 2025 | | CAGAUCUC | | | AUCUGGGA | |
| | | CCUCA | | | GGUGAAG | |
| BBD- | S26(53) | CACCUCCC | 1419_1439 | AS26(54) | UUGAGGGA | 1417_1439 |
| 2026 | | AGAUCUCC | | | GAUCUGGG | |
| | | CUCAA | | | AGGUGAA | |
| BBD- | S27(55) | CCAGUGUG | 1725_1745 | AS27(56) | UAGCUCCG | 1723_1745 |
| 2027 | | AGGACCGG | | | GUCCUCAC | |
| | | AGCUA | | | ACUGGAA | |
| BBD- | S28(57) | CACUGUGA | 1810_1830 | AS28(58) | UUGGAGGC | 1808_1830 |
| 2028 | | CUGUGGCC | | | CACAGUCA | |
| | | UCCAA | | | CAGUGCU | |
| BBD- | S29(59) | UUCGGGGU | 1904_1924 | AS29(60) | UACAGAUG | 1902_1924 |
| 2029 | | CGACACAU | | | UGUCGACC | |
| | | CUGUA | | | CCGAACC | |
| BBD- | S30(61) | UCGGGGUC | 1905_1925 | AS30(62) | UCACAGAU | 1903_1925 |
| 2030 | | GACACAUC | | | GUGUCGAC | |
| | | UGUGA | | | CCCGAAC | |
| BBD- | S31(63) | CGGGGUCG | 1906_1926 | AS31(64) | UCCACAGA | 1904_1926 |
| 2031 | | ACACAUCU | | | UGUGUCGA | |
| | | GUGGA | | | CCCCGAA | |
| BBD- | S32(65) | CCAGGAGG | 1974-1994 | AS32(66) | UAGGCCAU | 1972-1994 |
| 2032 | | ACAGCAUG | | | GCUGUCCU | |
| | | GCCUA | | | CCUGGAA | |
| BBD- | S33(67) | CUUCCAGG | 1971-1991 | AS33(68) | UCCAUGCU | 1969-1991 |
| 2033 | | AGGACAGC | | | GUCCUCCU | |
| | | AUGGA | | | GGAAGCA | |
| BBD- | S34(69) | GCUUCCAG | 1970-1990 | AS34(70) | UCAUGCUG | 1968-1990 |
| 2034 | | GAGGACAG | | | UCCUCCUG | |
| | | CAUGA | | | GAAGCAG | |
| BBD- | S35(71) | UGCUUCCA | 1969-1989 | AS35(72) | UAUGCUGU | 1967-1989 |
| 2035 | | GGAGGACA | | | CCUCCUGG | |
| | | GCAUA | | | AAGCAGU | |
| BBD- | S36(73) | CUGCUUCC | 1968-1988 | AS36(74) | AUGCUGUC | 1966-1988 |
| 2036 | | AGGAGGAC | | | CUCCUGGA | |
| | | AGCAU | | | AGCAGUG | |
| BBD- | S37(75) | GUGUCCUU | 2056-2076 | AS37(76) | UCGGCUCA | 2054-2076 |
| 2037 | | CAAGGUGA | | | CCUUGAAG | |
| | | GCCGA | | | GACACCU | |
| BBD- | S38(77) | GGUGUCCU | 2055-2075 | AS38(78) | UGGCUCAC | 2053-2075 |
| 2038 | | UCAAGGUG | | | CUUGAAGG | |
| | | AGCCA | | | ACACCUC | |
| BBD- | S39(79) | AGGUGUCC | 2054-2074 | AS39(80) | UGCUCACC | 2052-2074 |
| 2039 | | UUCAAGGU | | | UUGAAGGA | |
| | | GAGCA | | | CACCUCU | |
| BBD- | S40(81) | AGCCAUGA | 2107-2127 | AS40(82) | UGCCACGU | 2105-2127 |
| 2040 | | CUACGACG | | | CGUAGUCA | |
| | | UGGCA | | | UGGCUGU | |
| BBD- | S41(83) | CAGCCAUG | 2106-2126 | AS41(84) | UCCACGUC | 2104-2126 |
| 2041 | | ACUACGAC | | | GUAGUCAU | |
| | | GUGGA | | | GGCUGUC | |
| BBD- | S42(85) | ACAGCCAU | 2105-2125 | AS42(86) | UCACGUCG | 2103-2125 |
| 2042 | | GACUACGA | | | UAGUCAUG | |
| | | CGUGA | | | GCUGUCC | |
| BBD- | S43(87) | GACAGCCA | 2104-2124 | AS43(88) | UACGUCGU | 2102-2124 |
| 2043 | | UGACUACG | | | AGUCAUGG | |
| | | ACGUA | | | CUGUCCU | |
| BBD- | S44(89) | GGACAGCC | 2103-2123 | AS44(90) | ACGUCGUA | 2101-2123 |
| 2044 | | AUGACUAC | | | GUCAUGGC | |
| | | GACGU | | | UGUCCUC | |
| BBD- | S45(91) | AGGACAGC | 2102-2122 | AS45(92) | UGUCGUAG | 2100-2122 |
| 2045 | | CAUGACUA | | | UCAUGGCU | |
| | | CGACA | | | GUCCUCU | |
| BBD- | S46(93) | GAGGACAG | 2101-2122 | AS46(94) | UUCGUAGU | 2099-2121 |
| 2046 | | CCAUGACU | | | CAUGGCUG | |
| | | ACGAA | | | UCCUCUU | |
| BBD- | S47(95) | CGCCUGGG | 505_525 | AS47(96) | AUUGUAGU | 503_525 |
| 2047 | | AACUUACU | | | AAGUUCCC | |
| | | ACAAU | | | AGGCGGG | |
| BBD- | S48(97) | GCCUGGGA | 506_526 | AS48(98) | AGUUGUAG | 504_526 |
| 2048 | | ACUUACUA | | | UAAGUUCC | |
| | | CAACU | | | CAGGCGG | |
| BBD- | S49(99) | CCUGGGAA | 507_527 | AS49(100) | UAGUUGUA | 505_527 |
| 2049 | | CUUACUAC | | | GUAAGUUC | |
| | | AACUA | | | CCAGGCG | |
| BBD- | S50(101) | CUGGGAAC | 508_528 | AS50(102) | UGAGUUGU | 506_528 |
| 2050 | | UUACUACA | | | AGUAAGUU | |
| | | ACUCA | | | CCCAGGC | |
| BBD- | S51(103) | UGGGAACU | 509_529 | AS51(104) | UGGAGUUG | 507_529 |
| 2051 | | UACUACAA | | | UAGUAAGU | |
| | | CUCCA | | | UCCCAGG | |
| BBD- | S52(105) | GGGAACUU | 510_530 | AS52(106) | UUGGAGUU | 508_530 |
| 2052 | | ACUACAAC | | | GUAGUAAG | |
| | | UCCAA | | | UUCCCAG | |
| BBD- | S53(107) | GGAACUUA | 511_531 | AS53(108) | UCUGGAGU | 509_531 |
| 2053 | | CUACAACU | | | UGUAGUAA | |
| | | CCAGA | | | GUUCCCA | |
| BBD- | S54(109) | GAACUUAC | 512_532 | AS54(110) | AGCUGGAG | 510_532 |
| 2054 | | UACAACUC | | | UUGUAGUA | |
| | | CAGCU | | | AGUUCCC | |
| BBD- | S55(111) | AACUUACU | 513_533 | AS55(112) | UAGCUGGA | 511_533 |
| 2055 | | ACAACUCC | | | GUUGUAGU | |
| | | AGCUA | | | AAGUUCC | |
| BBD- | S56(113) | ACUUACUA | 514_534 | AS56(114) | UGAGCUGG | 512_534 |
| 2056 | | CAACUCCA | | | AGUUGUAG | |
| | | GCUCA | | | UAAGUUC | |
| BBD- | S57(115) | CUUACUAC | 515_535 | AS57(116) | UGGAGCUG | 513_535 |
| 2057 | | AACUCCAG | | | GAGUUGUA | |
| | | CUCCA | | | GUAAGUU | |
| BBD- | S58(117) | UUACUACA | 516_536 | AS58(118) | ACGGAGCU | 514_536 |
| 2058 | | ACUCCAGC | | | GGAGUUGU | |
| | | UCCGU | | | AGUAAGU | |
| BBD- | S59(119) | UACUACAA | 517_537 | AS59(120) | UACGGAGC | 515_537 |
| 2059 | | CUCCAGCU | | | UGGAGUUG | |
| | | CCGUA | | | UAGUAAG | |
| BBD- | S60(121) | ACUACAAC | 518_538 | AS60(122) | AGACGGAG | 516_538 |
| 2060 | | UCCAGCUC | | | CUGGAGUU | |
| | | CGUCU | | | GUAGUAA | |
| BBD- | S61(123) | CUACAACU | 519_539 | AS61(124) | UAGACGGA | 517_539 |
| 2061 | | CCAGCUCC | | | GCUGGAGU | |
| | | GUCUA | | | UGUAGUA | |
| BBD- | S62(125) | UACAACUC | 520_540 | AS62(126) | AUAGACGG | 518_540 |
| 2062 | | CAGCUCCG | | | AGCUGGAG | |
| | | UCUAU | | | UUGUAGU | |
| BBD- | S63(127) | ACAACUCC | 521_541 | AS63(128) | AAUAGACG | 519_541 |
| 2063 | | AGCUCCGU | | | GAGCUGGA | |
| | | CUAUU | | | GUUGUAG | |
| BBD- | S64(129) | CAACUCCA | 522_542 | AS64(130) | UAAUAGAC | 520_542 |
| 2064 | | GCUCCGUC | | | GGAGCUGG | |
| | | UAUUA | | | AGUUGUA | |
| BBD- | S65(131) | AACUCCAG | 523_543 | AS65(132) | UGAAUAGA | 521_543 |
| 2065 | | CUCCGUCU | | | CGGAGCUG | |
| | | AUUCA | | | GAGUUGU | |
| BBD- | S66(133) | ACUCCAGC | 524_544 | AS66(134) | AGGAAUAG | 522_544 |
| 2066 | | UCCGUCUA | | | ACGGAGCU | |
| | | UUCCU | | | GGAGUUG | |
| BBD- | S67(135) | CUCCAGCU | 525_545 | AS67(136) | AAGGAAUA | 523_545 |
| 2067 | | CCGUCUAU | | | GACGGAGC | |
| | | UCCUU | | | UGGAGUU | |
| BBD- | S68(137) | UCCAGGCC | 1112_1132 | AS68(138) | UGUUCACU | 1110_1132 |
| 2068 | | UGUGAAGU | | | UCACAGGC | |
| | | GAACA | | | CUGGAAG | |
| BBD- | S69(139) | CCAGGCCU | 1113_1133 | AS69(140) | AGGUUCAC | 1111_1133 |
| 2069 | | GUGAAGUG | | | UUCACAGG | |
| | | AACCU | | | CCUGGAA | |
| BBD- | S70(141) | CAGGCCUG | 1114_1134 | AS70(142) | UAGGUUCA | 1112_1134 |
| 2070 | | UGAAGUGA | | | CUUCACAG | |
| | | ACCUA | | | GCCUGGA | |
| BBD- | S71(143) | AGGCCUGU | 1115_1135 | AS71(144) | UCAGGUUC | 1113_1135 |
| 2071 | | GAAGUGAA | | | ACUUCACA | |
| | | CCUGA | | | GGCCUGG | |
| BBD- | S72(145) | GGCCUGUG | 1116_1136 | AS72(146) | UUCAGGUU | 1114_1136 |
| 2072 | | AAGUGAAC | | | CACUUCAC | |
| | | CUGAA | | | AGGCCUG | |
| BBD- | S73(147) | GCCUGUGA | 1117_1137 | AS73(148) | UGUCAGGU | 1115_1137 |
| 2073 | | AGUGAACC | | | UCACUUCA | |
| | | UGACA | | | CAGGCCU | |
| BBD- | S74(149) | CUGUGAAG | 1119_1139 | AS74(150) | AGCGUCAG | 1117_1139 |
| 2074 | | UGAACCUG | | | GUUCACUU | |
| | | ACGCU | | | CACAGGC | |
| BBD- | S75(151) | UGUGAAGU | 1120_1140 | AS75(152) | UAGCGUCA | 1118_1140 |
| 2075 | | GAACCUGA | | | GGUUCACU | |
| | | CGCUA | | | UCACAGG | |
| BBD- | S76(153) | GUGAAGUG | 1121_1141 | AS76(154) | UCAGCGUC | 1119_1141 |
| 2076 | | AACCUGAC | | | AGGUUCAC | |
| | | GCUGA | | | UUCACAG | |
| BBD- | S77(155) | AUCGCUGA | 1936_1956 | AS77(156) | UAUCACCC | 1934_1956 |
| 2077 | | CCGCUGGG | | | AGCGGUCA | |
| | | UGAUA | | | GCGAUGA | |
| BBD- | S78(157) | UCGCUGAC | 1937_1957 | AS78(158) | UUAUCACC | 1935_1957 |
| 2078 | | CGCUGGGU | | | CAGCGGUC | |
| | | GAUAA | | | AGCGAUG | |
| BBD- | S79(159) | CGCUGACC | 1938_1958 | AS79(160) | AUUAUCAC | 1936_1958 |
| 2079 | | GCUGGGUG | | | CCAGCGGU | |
| | | AUAAU | | | CAGCGAU | |
| BBD- | S80(161) | GCUGACCG | 1939_1959 | AS80(162) | UGUUAUCA | 1937_1959 |
| 2080 | | CUGGGUGA | | | CCCAGCGG | |
| | | UAACA | | | UCAGCGA | |
| BBD- | S81(163) | GACCGCUG | 1942_1962 | AS81(164) | AGCUGUUA | 1940_1962 |
| 2081 | | GGUGAUAA | | | UCACCCAG | |
| | | CAGCU | | | CGGUCAG | |
| BBD- | S82(165) | ACCGCUGG | 1943_1963 | AS82(166) | UAGCUGUU | 1941_1963 |
| 2082 | | GUGAUAAC | | | AUCACCCA | |
| | | AGCUA | | | GCGGUCA | |
| BBD- | S83(167) | UGCUACUC | 326-345 | AS83(168) | UUAGGAAA | 324-345 |
| 2083 | | UGGUAUUU | | | UACCAGAG | |
| | | CCUAA | | | UAGCACC | |
| BBD- | S84(169) | GUGCUACU | 325-345 | AS84(170) | UAGGAAAU | 323-345 |
| 2084 | | CUGGUAUU | | | ACCAGAGU | |
| | | UCCUA | | | AGCACCC | |
| BBD- | S85(171) | GGUGCUAC | 324-344 | AS85(172) | AGGAAAUA | 322-344 |
| 2085 | | UCUGGUAU | | | CCAGAGUA | |
| | | UUCCU | | | GCACCCC | |
| BBD- | S86(173) | GGGUGCUA | 323-343 | AS86(174) | UGAAAUAC | 321-343 |
| 2086 | | CUCUGGUA | | | CAGAGUAG | |
| | | UUUCA | | | CACCCCC | |
| BBD- | S87(175) | GGGGUGCU | 322-342 | AS87(176) | UAAAUACC | 320-342 |
| 2087 | | ACUCUGGU | | | AGAGUAGC | |
| | | AUUUA | | | ACCCCCG | |
| BBD- | S88(177) | GGGGGUGC | 321-341 | AS88(178) | AAAUACCA | 319-341 |
| 2088 | | UACUCUGG | | | GAGUAGCA | |
| | | UAUUU | | | CCCCCGC | |
| BBD- | S89(179) | CGGGGGUG | 320-340 | AS89(180) | AAUACCAG | 318-360 |
| 2089 | | CUACUCUG | | | AGUAGCAC | |
| | | GUAUU | | | CCCCGCC | |
| BBD- | S90(181) | GCUACUCU | 327-347 | AS90(182) | CCUAGGAA | 325-347 |
| 2090 | | GGUAUUUC | | | AUACCAGA | |
| | | CUAGG | | | GUAGCAC | |
| BBD- | S91(183) | CUACUCUG | 328-348 | AS91(184) | CCCUAGGA | 326-348 |
| 2091 | | GUAUUUCC | | | AAUACCAG | |
| | | UAGGG | | | AGUAGCA | |
| BBD- | S92(185) | UACUCUGG | 329-349 | AS92 (186) | ACCCUAGG | 327-349 |
| 2092 | | UAUUUCCU | | | AAAUACCA | |
| | | AGGGU | | | GAGUAGC | |
| AD- | (187) | UGCUACUC | 326-345 | (188) | AUAGGAAA | 324-345 |
| 1554911 | | UGGUAUUU | | | UACCAGAG | |
| | | CCUAU | | | UAGCACC | |

### Example 2: In Vitro Screening of siRNA Using Liposome Transfection in Hep3B Cells

Cell culture and transfection in 96-Well plate: In vitro experiments were conducted using Hep3B cells, which were cultured in MEM supplemented with 10% FBS, 1X penicillin-streptomycin, and 1X non-essential amino acids. When the cell confluence reached 80%, the cells were digested with trypsin and the cell concentration was measured using a Scepter automated cell counter (Millipore, #PHCC00000). Meanwhile, siRNA, Opti-MEM, and INTERFERin (Polyplus Transfection) were mixed and incubated in a 96-well plate and incubated at room temperature for 10 minutes. Subsequently, the mixture was added to each well containing Hep3B cells in complete culture medium, and the plate was incubated at 37° C. with 5% CO₂ for 24 hours.

Unmodified siRNA was screened at a final concentration of 1 nM.

RNA extraction and reverse transcription from a 96-well plate: mRNA was extracted from cells in the 96-well plate using the Dynabeads mRNA DIRECT Kit (Ambion). The culture medium was aspirated, and the wells were rinsed once with DPBS. Then, 50-300 μL of cell lysis buffer was added to each well, followed by 20-100 μL of beads. The plate was placed on a shaker for thorough mixing. Next, the 96-well plate was placed on a magnetic stand, and the lysis buffer was aspirated. Each well was then washed with 50-300 μL of Wash Buffer A, mixed thoroughly, and placed on the magnetic stand to remove the buffer. The beads were then resuspended in Wash Buffer B, transferred to a new 96-well plate, and placed on the magnetic stand to remove the buffer. The beads were once again resuspended in Wash Buffer B and transferred to a 96-well PCR plate. Meanwhile, reverse transcription reagents were prepared. The 96-well PCR plate was placed on a magnetic stand, and the wash buffer was aspirated. Then, 20 μL of reverse transcription reagent was added to each well, and the plate was sealed with sealing film. The plate was incubated at 25° C. for 10 minutes on a PCR instrument, then at 37° C. for 2 hours, followed by 85° C. for 5 minutes, and finally cooled to 4° C. to complete the reverse transcription process.

Real-time fluorescent quantitative PCR (qPCR): After reverse transcription, the 96-well plate was placed on a magnetic stand until the beads were completely adsorbed to the bottom. The reverse transcription reagent was then aspirated, and the prepared qPCR reaction mixture was added to the 96-well PCR plate. The plate was sealed with a sealing film and subjected to qPCR analysis using the StepOnePlus Real-Time PCR System (Applied Biosystems). 5

The ΔΔCt method was used to analyze the data, and the test was normalized using cells transfected with a 1 nM negative control sequence.

The negative control AD-1955 sequence was: Sense strand: CUUACGCUGAGUACUUCGAdTdT (SEQ ID NO.187) Antisense strand: UCGAAGUACUCAGCGUAAGdTdT (SEQ ID NO.188)

Table 2 shows the screening results of unmodified siRNA transfected at a concentration of 1 nM in Hep3B cells.

| | |
|---|---|
| | mRNA level in Hep3B cells (%) |
| siRNA | (relative to negative control cells) |

| Duplex | 1 nM avg | SD |
|---|---|---|
| TMPRSS6- | 25.85 | 4.68 |
| hcm-9 | | |
| BBD-2001 | 44.51 | 0.39 |
| BBD-2002 | 90.44 | 4.29 |
| BBD-2003 | 33.34 | 3.75 |
| BBD-2004 | 68.18 | 5.57 |
| BBD-2005 | 26.47 | 1.40 |
| BBD-2006 | 53.87 | 3.90 |
| BBD-2007 | 69.38 | 8.36 |
| BBD-2008 | 61.20 | 8.62 |
| BBD-2009 | 53.58 | 8.57 |
| BBD-2010 | 62.46 | 13.61 |
| BBD-2011 | 74.77 | 5.71 |
| BBD-2012 | 51.13 | 4.14 |
| BBD-2013 | 32.25 | 3.20 |
| BBD-2014 | 29.14 | 4.84 |
| BBD-2015 | 50.11 | 9.25 |
| BBD-2016 | 25.86 | 2.37 |
| BBD-2017 | 40.37 | 2.03 |
| BBD-2018 | 30.26 | 5.31 |
| BBD-2019 | 33.95 | 4.77 |
| BBD-2020 | 36.15 | 3.19 |
| BBD-2021 | 65.42 | 6.82 |
| BBD-2022 | 56.01 | 5.34 |
| BBD-2023 | 45.64 | 7.76 |
| BBD-2024 | 83.71 | 7.26 |
| BBD-2025 | 87.45 | 12.37 |
| BBD-2026 | 67.00 | 6.13 |
| BBD-2027 | 80.47 | 3.76 |
| BBD-2028 | 63.95 | 10.03 |
| BBD-2029 | 99.49 | 17.48 |
| BBD-2030 | 89.02 | 7.22 |
| BBD-2031 | 84.44 | 10.02 |
| BBD-2032 | 88.48 | 5.46 |
| BBD-2033 | 100.49 | 12.70 |
| BBD-2034 | 99.74 | 23.78 |
| BBD-2035 | 67.76 | 9.77 |
| BBD-2036 | 87.30 | 8.49 |
| BBD-2037 | 59.57 | 2.84 |
| BBD-2038 | 63.93 | 2.83 |
| BBD-2039 | 63.40 | 3.87 |
| BBD-2040 | 88.84 | 4.33 |
| BBD-2041 | 74.42 | 7.48 |
| BBD-2042 | 44.17 | 7.48 |
| BBD-2043 | 41.89 | 2.87 |
| BBD-2044 | 37.04 | 4.81 |
| BBD-2045 | 53.40 | 6.91 |
| BBD-2046 | 47.74 | 9.28 |
| BBD-2047 | 18.88 | 2.71 |
| BBD-2048 | 15.07 | 3.47 |
| BBD-2049 | 21.18 | 3.87 |
| BBD-2050 | 18.90 | 2.95 |
| BBD-2051 | 11.19 | 3.86 |
| BBD-2052 | 54.69 | 4.51 |
| BBD-2053 | 71.27 | 5.90 |
| BBD-2054 | 61.06 | 15.96 |
| BBD-2055 | 55.83 | 7.12 |
| BBD-2056 | 35.62 | 7.07 |
| BBD-2057 | 42.90 | 7.61 |
| BBD-2058 | 49.78 | 11.87 |
| BBD-2059 | 49.10 | 11.03 |
| BBD-2060 | 65.92 | 13.24 |
| BBD-2061 | 51.45 | 12.52 |
| BBD-2062 | 14.49 | 0.83 |
| BBD-2063 | 51.63 | 14.27 |
| BBD-2064 | 17.71 | 3.05 |
| BBD-2065 | 60.30 | 7.25 |
| BBD-2066 | 46.68 | 7.23 |
| BBD-2067 | 45.39 | 7.07 |
| BBD-2068 | 56.96 | 4.30 |
| BBD-2069 | 60.30 | 14.22 |
| BBD-2070 | 67.24 | 10.25 |
| BBD-2071 | 58.32 | 8.63 |
| BBD-2072 | 57.56 | 9.09 |
| BBD-2073 | 43.46 | 6.72 |
| BBD-2074 | 36.34 | 7.23 |
| BBD-2075 | 57.95 | 11.28 |
| BBD-2076 | 46.99 | 10.47 |
| BBD-2077 | 63.98 | 12.57 |
| BBD-2078 | 20.54 | 5.66 |
| BBD-2079 | 44.91 | 17.92 |
| BBD-2080 | 57.08 | 13.55 |
| BBD-2081 | 73.76 | 8.10 |
| BBD-2082 | 77.17 | 6.88 |
| BBD-2083 | 20.94 | 2.64 |

The experimental results indicate that the siRNA sequences in Table 1 inhibited TMPRSS6 expression in Hep3B cells at varying levels at a concentration of 1 nM. Following comparison and comprehensive evaluation, some of the more active sequences were selected for modification to enhance their in vivo stability and activity.

Table 3 describes multiple modified sense strand sequences of siRNA.

| modified sense strand ID | modified sense strand sequence 5′-3′ |
|---|---|
| TMPRSS6-hcm-9B | fUmCfAmCfCmUfGmCfUmUfCmUfUmCfUmGfG*mU*fU |
| BBD-2003SM1 | mG*mU*mGmAmUmCfCmUdGfGfAmAfGmCmCmAmGmUmGmUmA |
| BBD-2005SM1 | mG*mA*mUmCmCmUfGmGdAfAfGmCfCmAmGmUmGmUmGmAmA |
| BBD-2013SM1 | mU*mC*mAmCmCmAfUmCdAfAfCmUfUmCmAmCmCmUmCmCmA |
| BBD-2014SM1 | mC*mA*mCmCmAmUfCmAdAfCfUmUfCmAmCmCmUmCmCmCmA |
| BBD-2015SM1 | mA*mC*mCmAmUmCfAmAdCfUfUmCfAmCmCmUmCmCmCmAmA |
| BBD-2016SM1 | mC*mC*mAmUmCmAfAmCdTfUfCmAfCmCmUmCmCmCmAmGmA |
| BBD-2017SM1 | mC*mA*mUmCmAmAfCmUdTfCfAmCfCmUmCmCmCmAmGmAmU |
| BBD-2018SM1 | mA*mU*mCmAmAmCfUmUdCfAfCmCfUmCmCmCmAmGmAmUmA |
| BBD-2019SM1 | mU*mC*mAmAmCmUfUmCdAfCfCmUfCmCmCmAmGmAmUmCmU |
| BBD-2020SM1 | mC*mA*mAmCmUmUfCmAdCfCfUmCfCmCmAmGmAmUmCmUmA |
| BBD-2023SM1 | mC*mU*mUmCmAmCfCmUdCfCfCmAfGmAmUmCmUmCmCmCmU |
| BBD-2042SM1 | mA*mC*mAmGmCmCfAmUdGfAfCmUfAmCmGmAmCmGmUmGmA |
| BBD-2043SM1 | mG*mA*mCmAmGmCfCmAdTfGfAmCfUmAmCmGmAmCmGmUmA |
| BBD-2044SM1 | mG*mG*mAmCmAmGfCmCdAfUfGmAfCmUmAmCmGmAmCmGmU |
| BBD-2047SM1 | mC*mG*mCmCmUmGfGmGdAfAfCmUfUmAmCmUmAmCmAmAmU |
| BBD-2048SM1 | mG*mC*mCmUmGmGfGmAdAfCfUmUfAmCmUmAmCmAmAmCmU |
| BBD-2049SM1 | mC*mC*mUmGmGmGfAmAdCfUfUmAfCmUmAmCmAmAmCmUmA |
| BBD-2050SM1 | mC*mU*mGmGmGmAfAmCdTfUfAmCfUmAmCmAmAmCmUmCmA |
| BBD-2051SM1 | mU*mG*mGmGmAmAfCmUdTfAfCmUfAmCmAmAmCmUmCmCmA |
| BBD-2056SM1 | mA*mC*mUmUmAmCfUmAdCfAfAmCfUmCmCmAmGmCmUmCmA |
| BBD-2057SM1 | mC*mU*mUmAmCmUfAmCdAfAfCmUfCmCmAmGmCmUmCmCmA |
| BBD-2058SM1 | mU*mU*mAmCmUmAfCmAdAfCfUmCfCmAmGmCmUmCmCmGmU |
| BBD-2059SM1 | mU*mA*mCmUmAmCfAmAdCfUfCmCfAmGmCmUmCmCmGmUmA |
| BBD-2062SM1 | mU*mA*mCmAmAmCfUmCdCfAfGmCfUmCmCmGmUmCmUmAmU |
| BBD-2064SM1 | mC*mA*mAmCmUmCfCmAdGfCfUmCfCmGmUmCmUmAmUmUmA |
| BBD-2074SM1 | mC*mU*mGmUmGmAfAmGdTfGfAmAfCmCmUmGmAmCmGmCmU |
| BBD-2078SM1 | mU*mC*mGmCmUmGfAmCdCfGfCmUfGmGmGmUmGmAmUmAmA |
| BBD-2078SM2 | mU*mC*mGmCmUmGfAmCfCfGdCmUfGmGmGmUmGmAmUmAmA |
| BBD-2078SM3 | mU*mC*mGmCmUmGfAmCfCfGfCmUfGmGmGmUmGmAmUmAmA |
| BBD-2083SM1 | mU*mG*mCmUmAmCfUmCdTfGfGmUfAmUmUmUmCmCmUmAmA |
| BBD-2084SM1 | mG*mU*mGmCmUmAfCmUfCfUfGmGmUmAmUmUmUmCmCmUmA |
| BBD-2085SM1 | mG*mG*mUmGmCmUfAmCfUfCfUmGmGmUmAmUmUmUmCmCmU |
| BBD-2086SM1 | mG*mG*mGmUmGmCfUmAfCfUfCmUmGmGmUmAmUmUmUmCmA |
| BBD-2087SM1 | mG*mG*mGmGmUmGfCmUfAfCfUmCmUmGmGmUmAmUmUmUmA |
| BBD-2088SM1 | mG*mG*mGmGmGmUfGmCfUfAfCmUmCmUmGmGmUmAmUmUmU |
| BBD-2089SM1 | mC*mG*mGmGmGmGfUmGfCfUfAmCmUmCmUmGmGmUmAmUmU |
| BBD-2090SM1 | mG*mC*mUmAmCmUfCmUfGfGfUmAmUmUmUmCmCmUmAmGmG |
| BBD-2091SM1 | mC*mU*mAmCmUmCfUmGfGfUfAmUmUmUmCmCmUmAmGmGmG |
| BBD-2092SM1 | mU*mA*mCmUmCmUfGmGfUfAfUmUmUmCmCmUmAmGmGmGmU |

Table 4 describes multiple modified antisense strand sequences of siRNA.

| modified | |
|---|---|
| antisense strand ID | modified antisense strand sequence 5′-3′ |
| TMPRSS6-hcm-9A | mA*fA*mCfCmAfGmAfAmGfArnAfGmCfAmGfGmU*fG*mA |
| BBD-2003AM1 | mU*fA*mCmAmCmUmGmGmCfUmUdCmCfAmGfGmAmUmCmAmC* |
| | mU*mA |
| BBD-2005AM1 | mU*fU*mCmAmCmAmCmUmGfGmCdTmUfCmCfAmGmGmAmUmC* |
| | mA*mC |
| BBD-2013AM1 | mU*fG*mGmAmGmGmUmGmAfAmGdTmUfGmAfUmGmGmUmGmA |
| | *mU*mC |
| BBD-2014AM1 | mU*fG*mGmGmAmGmGmUmGfAmAdGmUfUmGfAmUmGmGmUmG |
| | *mA*mU |
| BBD-2015AM1 | mU*fU*mGmGmGmAmGmGmUfGmAdAmGfUmUfGmAmUmGmGmU |
| | *mG*mA |
| BBD-2016AM1 | mU*fC*mUmGmGmGmAmGmGfUmGdAmAfGmUfUmGmAmUmGmG |
| | *mU*mG |
| BBD-2017AM1 | mA*fU*mCmUmGmGmGmAmGfGmUdGmAfAmGfUmUmGmAmUmG |
| | *mG*mU |
| BBD-2018AM1 | mU*fA*mUmCmUmGmGmGmAfGmGdTmGfAmAfGmUmUmGmAmU |
| | *mG*mG |
| BBD-2019AM1 | mA*fG*mAmUmCmUmGmGmGfAmGdGmUfGmAfAmGmUmUmGmA |
| | *mU*mG |
| BBD-2020AM1 | mU*fA*mGmAmUmCmUmGmGfGmAdGmGfUmGfAmAmGmUmUmG |
| | *mA*mU |
| BBD-2023AM1 | mA*fG*mGmGmAmGmAmUmCfUmGdGmGfAmGfGmUmGmAmAmG |
| | *mU*mU |
| BBD-2042AM1 | mU*fC*mAmCmGmUmCmGmUfAmGdTmCfAmUfGmGmCmUmGmU* |
| | mC*mC |
| BBD-2043AM1 | mU*fA*mCmGmUmCmGmUmAfGmUdCmAfUmGfGmCmUmGmUmC |
| | *mC*mU |
| BBD-2044AM1 | mA*fC*mGmUmCmGmUmAmGfUmCdAmUfGmGfCmUmGmUmCmC |
| | *mU*mC |
| BBD-2047AM1 | mA*fU*mUmGmUmAmGmUmAfAmGdTmUfCmCfCmAmGmGmCmG |
| | *mG*mG |
| BBD-2048AM1 | mA*fG*mUmUmGmUmAmGmUfAmAdGmUfUmCfCmCmAmGmGmC |
| | *mG*mG |
| BBD-2049AM1 | mU*fA*mGmUmUmGmUmAmGfUmAdAmGfUmUfCmCmCmAmGmG |
| | *mC*mG |
| BBD-2050AM1 | mU*fG*mAmGmUmUmGmUmAfGmUdAmAfGmUfUmCmCmCmAmG |
| | *mG*mC |
| BBD-2051AM1 | mU*fG*mGmAmGmUmUmGmUfAmGdTmAfAmGfUmUmCmCmCmA |
| | *mG*mG |
| BBD-2056AM1 | mU*fG*mAmGmCmUmGmGmAfGmUdTmGfUmAfGmUmAmAmGmU |
| | *mU*mC |
| BBD-2057AM1 | mU*fG*mGmAmGmCmUmGmGfAmGdTmUfGmUfAmGmUmAmAmG |
| | *mU*mU |
| BBD-2058AM1 | mA*fC*mGmGmAmGmCmUmGfGmAdGmUfUmGfUmAmGmUmAmA |
| | *mG*mU |
| BBD-2059AM1 | mU*fA*mCmGmGmAmGmCmUfGmGdAmGfUmUfGmUmAmGmUmA |
| | *mA*mG |
| BBD-2062AM1 | mA*fU*mAmGmAmCmGmGmAfGmCdTmGfGmAfGmUmUmGmUmA |
| | *mG*mU |
| BBD-2064AM1 | mU*fA*mAmUmAmGmAmCmGfGmAdGmCfUmGfGmAmGmUmUmG |
| | *mU*mA |
| BBD-2074AM1 | mA*fG*mCmGmUmCmAmGmGfUmUdCmAfCmUfUmCmAmCmAmG |
| | *mG*mC |
| BBD-2078AM1 | mU*fU*mAmUmCmAmCmCmCfAmGdCmGfGmUfCmAmGmCmGmA* |
| | mU*mG |
| BBD-2078AM2 | VPU*fU*mAmUisoGNA-C |
| | mAmCmCmCfAmGdCmGfGmUfCmAmGmCmGmA*mU*mG |
| BBD-2078AM3 | VPU*fU*mAmUmCisoGNA-A |
| | mCmCmCfAmGdCmGfGmUfCmAmGmCmGmA*mU*mG |
| BBD-2078AM4 | VPU*fU*mAmUmCmAisoGNA-C |
| | mCmCfAmGdCmGfGmUfCmAmGmCmGmA*mU*mG |
| BBD-2083AM1 | mU*fU*mAmGmGmAmAmAmUfAmCdCmAfGmAfGmUmAmGmCmA |
| | *mC*mC |
| BBD-2084AM1 | mU*fA*mGmGmAmAmAmUmAfCmCdAmGfAmGfUmAmGmCmAmC |
| | *mC*mC |
| BBD-2085AM1 | mA*fG*mGmAmAmAmUmAmCfCmAdGmAfGmUfAmGmCmAmCmC |
| | *mC*mC |
| BBD-2086AM1 | mU*fG*mAmAmAmUmAmCmCfAmGdAmGfUmAfGmCmAmCmCmC |
| | *mC*mC |
| BBD-2087AM1 | mU*fA*mAmAmUmAmCmCmAfGmAdGmUfAmGfCmAmCmCmCmC* |
| | mC*mG |
| BBD-2088AM1 | mA*fA*mAmUmAmCmCmAmGfAmGdTmAfGmCfAmCmCmCmCmC* |
| | mG*mC |
| BBD-2089AM1 | mA*fA*mUmAmCmCmAmGmAfGmUdAmGfCmAfCmCmCmCmCmG* |
| | mC*mC |
| BBD-2090AM1 | mC*fC*mUmAmGmGmAmAmAfUmAdCmCfAmGfAmGmUmAmGmC |
| | *mA*mC |
| BBD-2091AM1 | mC*fC*mCmUmAmGmGmAmAfAmUdAmCfCmAfGmAmGmUmAmG |
| | *mC*mA |
| BBD-2092AM1 | mA*fC*mCmCmUmAmGmGmAfAmAdTmAfCmCfAmGmAmGmUmA* |
| | mG*mC |

Table 5 describes multiple modified double-stranded siRNA sequences.

| | modified | | modified | |
|---|---|---|---|---|
| Duplex | sense | modified sense | antisense | modified antisense |
| ID | strand ID | strand sequence 5′-3′ | strand ID | strand sequence 5′-3′ |
| TMPRSS6- | TMPRSS6- | fUmCfAmCfCmUfGmCfUmUf | TMPRSS6- | mA*fA*mCfCmAfGmAfAmGfArn |
| hcm-9 | hcm-9B | CmUfUmCfUmGfG*mU*fU | hcm-9A | AfGmCfAmGfGmU*fG*mA |
| TMPRSS6- | TMPRSS6- | mG*mU*mGmAmUmCfCmUd | TMPRSS6- | mU*fA*mCmAmCmUmGmGmCf |
| hcm-9 | hcm-9B | GfGfAmAfGmCmCmAmGmU | hcm-9A | UmUdCmCfAmGfGmAmUmCmA |
| | | mGmUmA | | mC*mU*mA |
| BBD- | BBD-2005 | mG*mA*mUmCmCmUfGmGd | BBD-2005 | mU*fU*mCmAmCmAmCmUmGf |
| 2005.11 | SM1 | AfAfGmCfCmAmGmUmGmU | AM1 | GmCdTmUfCmCfAmGmGmAmU |
| | | mGmAmA | | mC*mA*mC |
| BBD- | BBD-2013 | mU*mC*mAmCmCmAfUmCd | BBD-2013 | mU*fG*mGmAmGmGmUmGmAf |
| 2013.11 | DM1 | AfAfCmUfUmCmAmCmCmU | AM1 | AmGdTmUfGmAfUmGmGmUmG |
| | | mCmCmA | | mA*mU*mC |
| BBD- | BBD- | mC*mA*mCmCmAmUfCmAd | BBD-2014 | mU*fG*mGmGmAmGmGmUmGf |
| 2014.11 | 2014 | AfCfUmUfCmAmCmCmUmC | AM1 | AmAdGmUfUmGfAmUmGmGmU |
| | SM1 | mCmCmA | | mG*mA*mU |
| BBD- | BBD- | mA*mC*mCmAmUmCfAmAd | BBD-2015 | mU*fU*mGmGmGmAmGmGmUf |
| 2015.11 | 2015 | CfUfUmCfAmCmCmUmCmC | AM1 | GmAdAmGfUmUfGmAmUmGmG |
| | SM1 | mCmAmA | | mU*mG*mA |
| BBD- | BBD- | mC*mC*mAmUmCmAfAmCd | BBD-2016 | mU*fC*mUmGmGmGmAmGmGf |
| 2016.11 | 2016 | TfUfCmAfCmCmUmCmCmC | AM1 | UmGdAmAfGmUfUmGmAmUmG |
| | SM1 | mAmGmA | | mG*mU*mG |
| BBD- | BBD- | mC*mA*mUmCmAmAfCmUd | BBD-2017 | mA*fU*mCmUmGmGmGmAmGf |
| 2017.11 | 2017 | TfCfAmCfCmUmCmCmCmA | AM1 | GmUdGmAfAmGfUmUmGmAmU |
| | SM1 | mGmAmU | | mG*mG*mU |
| BBD- | BBD- | mA*mU*mCmAmAmCfUmUd | BBD-2018 | mU*fA*mUmCmUmGmGmGmAf |
| 2018.11 | 2018 | CfAfCmCfUmCmCmCmAmG | AM1 | GmGdTmGfAmAfGmUmUmGmA |
| | SM1 | mAmUmA | | mU*mG*mG |
| BBD- | BBD- | mU*mC*mAmAmCmUfUmCd | BBD-2019 | mA*fG*mAmUmCmUmGmGmGf |
| 2019.11 | 2019 | AfCfCmUfCmCmCmAmGmA | AM1 | AmGdGmUfGmAfAmGmUmUmG |
| | SM1 | mUmCmU | | mA*mU*mG |
| BBD- | BBD- | mC*mA*mAmCmUmUfCmAd | BBD-2020 | mU*fA*mGmAmUmCmUmGmGf |
| 2020.11 | 2020 | CfCfUmCfCmCmAmGmAmU | AM1 | GmAdGmGfUmGfAmAmGmUmU |
| | SM1 | mCmUmA | | mG*mA*mU |
| BBD- | BBD- | mC*mU*mUmCmAmCfCmUd | BBD-2023 | mA*fG*mGmGmAmGmAmUmCf |
| 2023.11 | 2023 | CfCfCmAfGmAmUmCmUmC | AM1 | UmGdGmGfAmGfGmUmGmAmA |
| | SM1 | mCmCmU | | mG*mU*mU |
| BBD- | BBD- | mA*mC*mAmGmCmCfAmUd | BBD-2042 | mU*fC*mAmCmGmUmCmGmUf |
| 2042.11 | 2042 | GfAfCmUfAmCmGmAmCmG | AM1 | AmGdTmCfAmUfGmGmCmUmG |
| | SM1 | mUmGmA | | mU*mC*mC |
| BBD- | BBD- | mG*mA*mCmAmGmCfCmAd | BBD-2043 | mU*fA*mCmGmUmCmGmUmAf |
| 2043.11 | 2043 | TfGfAmCfUmAmCmGmAmC | AM1 | GmUdCmAfUmGfGmCmUmGmU |
| | SM1 | mGmUmA | | mC*mC*mU |
| BBD- | BBD- | mG*mG*mAmCmAmGfCmCd | BBD-2044 | mA*fC*mGmUmCmGmUmAmGf |
| 2044.11 | 2044 | AfUfGmAfCmUmAmCmGmA | AM1 | UmCdAmUfGmGfCmUmGmUmC |
| | SM1 | mCmGmU | | mC*mU*mC |
| BBD- | BBD- | mC*mG*mCmCmUmGfGmGd | BBD-2047 | mA*fU*mUmGmUmAmGmUmAf |
| 2047.11 | 2047 | AfAfCmUfUmAmCmUmAmC | AM1 | AmGdTmUfCmCfCmAmGmGmC |
| | SM1 | mAmAmU | | mG*mG*mG |
| BBD- | BBD- | mG*mC*mCmUmGmGfGmAd | BBD-2048 | mA*fG*mUmUmGmUmAmGmUf |
| 2048.11 | 2048 | AfCfUmUfAmCmUmAmCmA | AM1 | AmAdGmUfUmCfCmCmAmGmG |
| | SM1 | mAmCmU | | mC*mG*mG |
| BBD- | BBD- | mC*mC*mUmGmGmGfAmAd | BBD-2049 | mU*fA*mGmUmUmGmUmAmGf |
| 2049.11 | 2049 | CfUfUmAfCmUmAmCmAmA | AM1 | UmAdAmGfUmUfCmCmCmAmG |
| | SM1 | mCmUmA | | mG*mC*mG |
| BBD- | BBD- | mC*mU*mGmGmGmAfAmCd | BBD-2050 | mU*fG*mAmGmUmUmGmUmAf |
| 2050.11 | 2050 | TfUfAmCfUmAmCmAmAmC | AM1 | GmUdAmAfGmUfUmCmCmCmA |
| | SM1 | mUmCmA | | mG*mG*mC |
| BBD- | BBD- | mU*mG*mGmGmAmAfCmUd | BBD-2051 | mU*fG*mGmAmGmUmUmGmUf |
| 2051.11 | 2051 | TfAfCmUfAmCmAmAmCmU | AM1 | AmGdTmAfAmGfUmUmCmCmC |
| | SM1 | mCmCmA | | mA*mG*mG |
| BBD- | BBD- | mA*mC*mUmUmAmCfUmAd | BBD-2056 | mU*fG*mAmGmCmUmGmGmAf |
| 2056.11 | 2056 | CfAfAmCfUmCmCmAmGmC | AM1 | GmUdTmGfUmAfGmUmAmAmG |
| | SM1 | mUmCmA | | mU*mU*mC |
| BBD- | BBD- | mC*mU*mUmAmCmUfAmCd | BBD-2057 | mU*fG*mGmAmGmCmUmGmGf |
| 2057.11 | 2057 | AfAfCmUfCmCmAmGmCmU | AM1 | AmGdTmUfGmUfAmGmUmAmA |
| | SM1 | mCmCmA | | mG*mU*mU |
| BBD- | BBD- | mU*mU*mAmCmUmAfCmAd | BBD-2058 | mA*fC*mGmGmAmGmCmUmGf |
| 2058.11 | 2058 | AfCfUmCfCmAmGmCmUmC | AM1 | GmAdGmUfUmGfUmAmGmUmA |
| | SM1 | mCmGmU | | mA*mG*mU |
| BBD- | BBD- | mU*mA*mCmUmAmCfAmAd | BBD-2059 | mU*fA*mCmGmGmAmGmCmUf |
| 2059.11 | 2059 | CfUfCmCfAmGmCmUmCmC | AM1 | GmGdAmGfUmUfGmUmAmGmU |
| | SM1 | mGmUmA | | mA*mA*mG |
| BBD- | BBD- | mU*mA*mCmAmAmCfUmCd | BBD-2062 | mA*fU*mAmGmAmCmGmGmAf |
| 2062.11 | 2062 | CfAfGmCfUmCmCmGmUmC | AM1 | GmCdTmGfGmAfGmUmUmGmU |
| | SM1 | mUmAmU | | mA*mG*mU |
| BBD- | BBD- | mC*mA*mAmCmUmCfCmAd | BBD-2064 | mU*fA*mAmUmAmGmAmCmGf |
| 2064.11 | 2064 | GfCfUmCfCmGmUmCmUmA | AM1 | GmAdGmCfUmGfGmAmGmUmU |
| | SM1 | mUmUmA | | mG*mU*mA |
| BBD- | BBD- | mC*mU*mGmUmGmAfAmGd | BBD-2074 | mA*fG*mCmGmUmCmAmGmGf |
| 2074.11 | 2074 | TfGfAmAfCmCmUmGmAmC | AM1 | UmUdCmAfCmUfUmCmAmCmA |
| | SM1 | mGmCmU | | mG*mG*mC |
| BBD- | BBD- | mU*mC*mGmCmUmGfAmCd | BBD-2078 | mU*fU*mAmUmCmAmCmCmCf |
| 2078.11 | 2078 | CfGfCmUfGmGmGmUmGmA | AM1 | AmGdCmGfGmUfCmAmGmCmG |
| | SM1 | mUmAmA | | mA*mU*mG |
| BBD- | BBD- | mU*mC*mGmCmUmGfAmCd | BBD-2078 | VPU*fU*mAmUisoGNA-C |
| 2078.12 | 2078 | CfGfCmUfGmGmGmUmGmA | AM2 | mAmCmCmCfAmGdCmGfGmUfC |
| | SM1 | mUmAmA | | mAmGmCmGmA*mU*mG |
| BBD- | BBD- | mU*mC*mGmCmUmGfAmCd | BBD-2078 | VPU*fU*mAmUmCisoGNA-A |
| 2078.13 | 2078 | CfGfCmUfGmGmGmUmGmA | AM3 | mCmCmCfAmGdCmGfGmUfCmA |
| | SM1 | mUmAmA | | mGmCmGmA*mU*mG |
| BBD- | BBD- | mU*mC*mGmCmUmGfAmCd | BBD-2078 | VPU*fU*mAmUmCmAisoGNA-C |
| 2078.14 | 2078 | CfGfCmUfGmGmGmUmGmA | AM4 | mCmCfAmGdCmGfGmUfCmAmG |
| | SM1 | mUmAmA | | mCmGmA*mU*mG |
| BBD- | BBD- | mU*mC*mGmCmUmGfAmCf | BBD-2078 | mU*fU*mAmUmCmAmCmCmCf |
| 2078.21 | 2078 | CfGdCmUfGmGmGmUmGmA | AM1 | AmGdCmGfGmUfCmAmGmCmG |
| | SM2 | mUmAmA | | mA*mU*mG |
| BBD- | BBD- | mU*mC*mGmCmUmGfAmCf | BBD-2078 | VPU*fU*mAmUisoGNA-C |
| 2078.22 | 2078 | CfGdCmUfGmGmGmUmGmA | AM2 | mAmCmCmCfAmGdCmGfGmUfC |
| | SM2 | mUmAmA | | mAmGmCmGmA*mU*mG |
| BBD- | BBD- | mU*mC*mGmCmUmGfAmCf | BBD-2078 | VPU*fU*mAmUmCisoGNA-A |
| 2078.23 | 2078 | CfGdCmUfGmGmGmUmGmA | AM3 | mCmCmCfAmGdCmGfGmUfCmA |
| | SM2 | mUmAmA | | mGmCmGmA*mU*mG |
| BBD- | BBD- | mU*mC*mGmCmUmGfAmCf | BBD-2078 | VPU*fU*mAmUmCmAisoGNA-C |
| 2078.24 | 2078 | CfGdCmUfGmGmGmUmGmA | AM4 | mCmCfAmGdCmGfGmUfCmAmG |
| | SM2 | mUmAmA | | mCmGmA*mU*mG |
| BBD- | BBD- | mU*mC*mGmCmUmGfAmCf | BBD-2078 | mU*fU*mAmUmCmAmCmCmCf |
| 2078.31 | 2078 | CfGfCmUfGmGmGmUmGmA | AM1 | AmGdCmGfGmUfCmAmGmCmG |
| | SM3 | mUmAmA | | mA*mU*mG |
| BBD- | BBD- | mU*mC*mGmCmUmGfAmCf | BBD-2078 | VPU*fU*mAmUisoGNA-C |
| 2078.32 | 2078 | CfGfCmUfGmGmGmUmGmA | AM2 | mAmCmCmCfAmGdCmGfGmUfC |
| | SM3 | mUmAmA | | mAmGmCmGmA*mU*mG |
| BBD- | BBD- | mU*mC*mGmCmUmGfAmCf | BBD-2078 | VPU*fU*mAmUmCisoGNA-A |
| 2078.33 | 2078 | CfGfCmUfGmGmGmUmGmA | AM3 | mCmCmCfAmGdCmGfGmUfCmA |
| | SM3 | mUmAmA | | mGmCmGmA*mU*mG |
| BBD- | BBD- | mU*mC*mGmCmUmGfAmCf | BBD-2078 | VPU*fU*mAmUmCmAisoGNA-C |
| 2078.34 | 2078 | CfGfCmUfGmGmGmUmGmA | AM4 | mCmCfAmGdCmGfGmUfCmAmG |
| | SM3 | mUmAmA | | mCmGmA*mU*mG |
| BBD- | BBD- | mU*mG*mCmUmAmCfUmCd | BBD-2083 | mU*fU*mAmGmGmAmAmAmUf |
| 2083.11 | 2083 | TfGfGmUfAmUmUmUmCmC | AM1 | AmCdCmAfGmAfGmUmAmGmC |
| | SM1 | mUmAmA | | mA*mC*mC |
| BBD- | BBD- | mG*mU*mGmCmUmAfCmUf | BBD-2084 | mU*fA*mGmGmAmAmAmUmAf |
| 2084.11 | 2084 | CfUfGmGmUmAmUmUmUm | AM1 | CmCdAmGfAmGfUmAmGmCmA |
| | SM1 | CmCmUmA | | mC*mC*mC |
| BBD- | BBD- | mG*mG*mUmGmCmUfAmCf | BBD-2085 | mA*fG*mGmAmAmAmUmAmCf |
| 2085.11 | 2085 | UfCfUmGmGmUmAmUmUm | AM1 | CmAdGmAfGmUfAmGmCmAmC |
| | SM1 | UmCmCmU | | mC*mC*mC |
| BBD- | BBD- | mG*mG*mGmUmGmCfUmAf | BBD-2086 | mU*fG*mAmAmAmUmAmCmCf |
| 2086.11 | 2086 | CfUfCmUmGmGmUmAmUm | AM1 | AmGdAmGfUmAfGmCmAmCmC |
| | SM1 | UmUmCmA | | mC*mC*mC |
| BBD- | BBD- | mG*mG*mGmGmUmGfCmUf | BBD-2087 | mU*fA*mAmAmUmAmCmCmAf |
| 2087.11 | 2087 | AfCfUmCmUmGmGmUmAm | AM1 | GmAdGmUfAmGfCmAmCmCmC |
| | SM1 | UmUmUmA | | mC*mC*mG |
| BBD- | BBD- | mG*mG*mGmGmGmUfGmCf | BBD-2088 | mA*fA*mAmUmAmCmCmAmGf |
| 2088.11 | 2088 | UfAfCmUmCmUmGmGmUm | AM1 | AmGdTmAfGmCfAmCmCmCmC |
| | SM1 | AmUmUmU | | mC*mG*mC |
| BBD- | BBD- | mC*mG*mGmGmGmGfUmGf | BBD-2089 | mA*fA*mUmAmCmCmAmGmAf |
| 2089.11 | 2089 | CfUfAmCmUmCmUmGmGm | AM1 | GmUdAmGfCmAfCmCmCmCmC |
| | SM1 | UmAmUmU | | mG*mC*mC |
| BBD- | BBD- | mG*mC*mUmAmCmUfCmUf | BBD-2090 | mC*fC*mUmAmGmGmAmAmAf |
| 2090.11 | 2090 | GfGfUmAmUmUmUmCmCm | AM1 | UmAdCmCfAmGfAmGmUmAmG |
| | SM1 | UmAmGmG | | mC*mA*mC |
| BBD- | BBD- | mC*mU*mAmCmUmCfUmGf | BBD-2091 | mC*fC*mCmUmAmGmGmAmAf |
| 2091.11 | 2091 | GfUfAmUmUmUmCmCmUm | AM1 | AmUdAmCfCmAfGmAmGmUmA |
| | SM1 | AmGmGmG | | mG*mC*mA |
| BBD- | BBD- | mU*mA*mCmUmCmUfGmGf | BBD-2092 | mA*fC*mCmCmUmAmGmGmAf |
| 2092.11 | 2092 | UfAfUmUmUmCmCmUmAm | AM1 | AmAdTmAfCmCfAmGmAmGmU |
| | SM1 | GmGmGmU | | mA*mG*mC |

Table 6 describes the experimental results of modified double-stranded siRNA screened at 1 nM transfection concentration in Hep3B cells.

| | |
|---|---|
| | mRNA level in Hep3B cells (%) |
| siRNA | (relative to negative control cells) |

| Duplex | 1 nM avg | SD |
|---|---|---|
| TMPRSS6-hcm-9 | 47.00 | 16.38 |
| BBD-2003.11 | 81.59 | 1.17 |
| BBD-2005.11 | 58.15 | 1.80 |
| BBD-2013.11 | 64.28 | 12.64 |
| BBD-2014.11 | 72.25 | 10.82 |
| BBD-2015.11 | 57.40 | 2.93 |
| BBD-2016.11 | 94.41 | 4.46 |
| BBD-2017.11 | 54.37 | 7.90 |
| BBD-2018.11 | 62.84 | 6.74 |
| BBD-2019.11 | 60.63 | 8.20 |
| BBD-2020.11 | 62.93 | 2.90 |
| BBD-2023.11 | 66.26 | 15.88 |
| BBD-2042.11 | 80.91 | 1.56 |
| BBD-2043.11 | 53.02 | 4.03 |
| BBD-2044.11 | 81.53 | 17.27 |
| BBD-2047.11 | 35.02 | 9.98 |
| BBD-2048.11 | 42.86 | 6.25 |
| BBD-2049.11 | 56.69 | 23.22 |
| BBD-2050.11 | 41.65 | 11.26 |

Table 7 describes the experimental results of modified double-stranded siRNA screened at 10 nM transfection concentration in Hep3B cells.

| | |
|---|---|
| | mRNA level in Hep3B cells (%) |
| siRNA | (relative to negative control cells) |

| Duplex | 10 nM avg | SD |
|---|---|---|
| TMPRSS6-hcm-9 | 24.30 | 3.93 |
| BBD-2005.11 | 36.69 | 4.05 |
| BBD-2015.11 | 32.09 | 8.36 |
| BBD-2017.11 | 32.70 | 2.87 |
| BBD-2019.11 | 33.95 | 5.49 |
| BBD-2043.11 | 43.49 | 2.69 |
| BBD-2047.11 | 20.24 | 1.84 |
| BBD-2048.11 | 23.29 | 2.37 |
| BBD-2049.11 | 24.35 | 4.23 |
| BBD-2050.11 | 22.60 | 2.49 |
| BBD-2051.11 | 19.69 | 2.09 |
| BBD-2056.11 | 34.01 | 8.13 |
| BBD-2057.11 | 35.74 | 4.25 |
| BBD-2058.11 | 31.67 | 6.16 |
| BBD-2059.11 | 26.23 | 1.98 |
| BBD-2062.11 | 20.22 | 2.50 |
| BBD-2064.11 | 15.02 | 1.88 |
| BBD-2074.11 | 35.48 | 1.93 |
| BBD-2078.11 | 18.56 | 1.59 |
| BBD-2083.11 | 25.09 | 3.81 |
| BBD-2084.11 | 61.88 | 9.44 |
| BBD-2085.11 | 67.91 | 3.21 |
| BBD-2086.11 | 22.26 | 3.33 |
| BBD-2087.11 | 26.69 | 1.43 |
| BBD-2088.11 | 49.22 | 3.00 |
| BBD-2089.11 | 45.02 | 4.75 |
| BBD-2090.11 | 104.19 | 16.66 |
| BBD-2091.11 | 155.06 | 15.06 |
| BBD-2092.11 | 75.44 | 2.58 |

Next, the three sequences BBD-2051.11, BBD-2083.11, and BBD-2047.11, with relatively high activity, were further modified to identify the most optimal modification pattern.

Table 8 describes multiple modified sense strand sequences of BBD-2051.

| modified | |
|---|---|
| sense | |
| strand ID | modified sense strand sequence 5′-3′ |
| BBD-2051SM1 | mU*mG*mGmGmAmAfCmUdTfAfCmUfAmCmAmAm |
| | CmUmCmCmA |
| BBD-2051SM2 | mU*mG*mGmGmAmAfCmUfUfAfCmUmAmCmAmAm |
| | CmUmCmCmA |
| BBD-2051SM3 | mU*mG*mGmGmAmAfCmUfUfAdCmUmAmCmAmAm |
| | CmUmCmCmA |
| BBD-2051SM4 | mU*mG*mGmGmAmAfCmUfUdAfCmUmAmCmAmAm |
| | CmUmCmCmA |
| BBD-2051SM5 | mU*mG*mGmGmAmAfCmUdTfAfCmUmAmCmAmAm |
| | CmUmCmCmA |

Table 9 describes multiple modified antisense strand sequences of BBD-2051.

| modified | |
|---|---|
| antisense strand ID | modified antisense strand sequence 5′-3′ |
| BBD- | mU*fG*mGmAmGmUmUmGmUfAmGdTmAfAmGfUmUmCmCmCmA*m |
| 2051AM1 | G*mG |
| BBD- | mU*fG*mGmAmGmUmUmGmUfAdGmUmAfAmGfUmUmCmCmCmA*m |
| 2051AM2 | G*mG |
| BBD- | mU*fG*mGmAmGmUmUmGmUfAmGmUdAfAmGfUmUmCmCmCmA*m |
| 2051AM3 | G*mG |
| BBD- | mU*fG*mGmAmGmUmUmGmUfAmGmUmAfAmGfUmUmCmCmCmA* |
| 2051AM4 | mG*mG |
| BBD- | mU*fG*mGmAisoGNA- |
| 2051AM5 | GmUmUmGmUfAmGdTmAfAmGfUmUmCmCmCmA*mG*mG |
| BBD- | mU*fG*mGmAmGisoGNA- |
| 2051AM6 | TmUmGmUfAmGdTmAfAmGfUmUmCmCmCmA*mG*mG |
| BBD- | mU*fG*mGmAmGmUisoGNA- |
| 2051AM7 | TmGmUfAmGdTmAfAmGfUmUmCmCmCmA*mG*mG |
| BBD- | VPU*fG*mGmAmGmUmUmGmUfAmGdTmAfAmGfUmUmCmCmCmA* |
| 2051AM8 | mG*mG |
| BBD- | VPU*fG*mGmAisoGNA- |
| 2051AM9 | GmUmUmGmUfAmGdTmAfAmGfUmUmCmCmCmA*mG*mG |
| BBD- | VPU*fG*mGmAmGisoGNA- |
| 2051AM10 | TmUmGmUfAmGdTmAfAmGfUmUmCmCmCmA*mG*mG |
| BBD- | VPU*fG*mGmAmGmUisoGNA- |
| 2051AM11 | TmGmUfAmGdTmAfAmGfUmUmCmCmCmA*mG*mG |
| BBD- | mU*dG*mGmAmGmUmUmGmUmAmGdTmAfAmGfUmUmCmCmCmA* |
| 2051AM12 | mG*mG |
| BBD- | mU*dG*mGmAisoGNA- |
| 2051AM13 | GmUmUmGmUmAmGdTmAfAmGfUmUmCmCmCmA*mG*mG |
| BBD- | mU*dG*mGmAmGisoGNA- |
| 2051AM14 | TmUmGmUmAmGdTmAfAmGfUmUmCmCmCmA*mG*mG |
| BBD- | mU*dG*mGmAmGmUisoGNA- |
| 2051AM15 | TmGmUmAmGdTmAfAmGfUmUmCmCmCmA*mG*mG |
| BBD- | VPU*dG*mGmAmGmUmUmGmUmAmGdTmAfAmGfUmUmCmCmCmA |
| 2051AM16 | *mG*mG |
| BBD- | VPU*dG*mGmAisoGNA- |
| 2051AM17 | GmUmUmGmUmAmGdTmAfAmGfUmUmCmCmCmA*mG*mG |
| BBD- | VPU*dG*mGmAmGisoGNA- |
| 2051AM18 | TmUmGmUmAmGdTmAfAmGfUmUmCmCmCmA*mG*mG |
| BBD- | VPU*dG*mGmAmGmUisoGNA- |
| 2051AM19 | TmGmUmAmGdTmAfAmGfUmUmCmCmCmA*mG*mG |

Table 10 describes multiple modified double-stranded siRNA sequences of BBD-2051.

| | modified | | modified | |
|---|---|---|---|---|
| | sense | modified sense strand | antisense | modified antisense strand |
| duplex ID | strand ID | sequence 5′-3′ | strand ID | sequence 5′-3′ |
| BBD- | BBD- | mU*mG*mGmGmAmAfC | BBD- | mU*fG*mGmAmGmUmUmGmUfA |
| 2051.2 | 2051SM | mUfUfAfCmUmAmCmA | 2051A | mGdTmAfAmGfUmUmCmCmCmA |
| 1 | 2 | mAmCmUmCmCmA | M1 | *mG*mG |
| BBD- | BBD- | mU*mG*mGmGmAmAfC | BBD- | mU*fG*mGmAisoGNA- |
| 2051.2 | 2051SM | mUfUfAfCmUmAmCmA | 2051A | GmUmUmGmUfAmGdTmAfAmGf |
| 5 | 2 | mAmCmUmCmCmA | M5 | UmUmCmCmCmA*mG*mG |
| BBD- | BBD- | mU*mG*mGmGmAmAfC | BBD- | mU*fG*mGmAmGisoGNA- |
| 2051.2 | 2051SM | mUfUfAfCmUmAmCmA | 2051A | TmUmGmUfAmGdTmAfAmGfUm |
| 6 | 2 | mAmCmUmCmCmA | M6 | UmCmCmCmA*mG*mG |
| BBD- | BBD- | mU*mG*mGmGmAmAfC | BBD- | mU*fG*mGmAmGmUisoGNA- |
| 2051.2 | 2051SM | mUfUfAfCmUmAmCmA | 2051A | TmGmUfAmGdTmAfAmGfUmUm |
| 7 | 2 | mAmCmUmCmCmA | M7 | CmCmCmA*mG*mG |
| BBD- | BBD- | mU*mG*mGmGmAmAfC | BBD- | VPU*fG*mGmAmGmUmUmGmUf |
| 2051.2 | 2051SM | mUfUfAfCmUmAmCmA | 2051A | AmGdTmAfAmGfUmUmCmCmCm |
| 8 | 2 | mAmCmUmCmCmA | M8 | A*mG*mG |
| BBD- | BBD- | mU*mG*mGmGmAmAfC | BBD- | VPU*fG*mGmAisoGNA- |
| 2051.2 | 2051SM | mUfUfAfCmUmAmCmA | 2051A | GmUmUmGmUfAmGdTmAfAmGf |
| 9 | 2 | mAmCmUmCmCmA | M9 | UmUmCmCmCmA*mG*mG |
| BBD- | BBD- | mU*mG*mGmGmAmAfC | BBD- | VPU*fG*mGmAmGisoGNA- |
| 2051.2 | 2051SM | mUfUfAfCmUmAmCmA | 2051A | TmUmGmUfAmGdTmAfAmGfUm |
| 10 | 2 | mAmCmUmCmCmA | M10 | UmCmCmCmA*mG*mG |
| BBD- | BBD- | mU*mG*mGmGmAmAfC | BBD- | VPU*fG*mGmAmGmUisoGNA- |
| 2051.2 | 2051SM | mUfUfAfCmUmAmCmA | 2051A | TmGmUfAmGdTmAfAmGfUmUm |
| 11 | 2 | mAmCmUmCmCmA | M11 | CmCmCmA*mG*mG |
| BBD- | BBD- | mU*mG*mGmGmAmAfC | BBD- | mU*dG*mGmAmGmUmUmGmUm |
| 2051.2 | 2051SM | mUfUfAfCmUmAmCmA | 2051A | AmGdTmAfAmGfUmUmCmCmCm |
| 12 | 2 | mAmCmUmCmCmA | M12 | A*mG*mG |
| BBD- | BBD- | mU*mG*mGmGmAmAfC | BBD- | mU*dG*mGmAisoGNA- |
| 2051.2 | 2051SM | mUfUfAfCmUmAmCmA | 2051A | GmUmUmGmUmAmGdTmAfAmGf |
| 13 | 2 | mAmCmUmCmCmA | M13 | UmUmCmCmCmA*mG*mG |
| BBD- | BBD- | mU*mG*mGmGmAmAfC | BBD- | mU*dG*mGmAmGisoGNA- |
| 2051.2 | 2051SM | mUfUfAfCmUmAmCmA | 2051A | TmUmGmUmAmGdTmAfAmGfUm |
| 14 | 2 | mAmCmUmCmCmA | M14 | UmCmCmCmA*mG*mG |
| BBD- | BBD- | mU*mG*mGmGmAmAfC | BBD- | mU*dG*mGmAmGmUisoGNA- |
| 2051.2 | 2051SM | mUfUfAfCmUmAmCmA | 2051A | TmGmUmAmGdTmAfAmGfUmUm |
| 15 | 2 | mAmCmUmCmCmA | M15 | CmCmCmA*mG*mG |
| BBD- | BBD- | mU*mG*mGmGmAmAfC | BBD- | VPU*dG*mGmAmGmUmUmGmU |
| 2051.2 | 2051SM | mUfUfAfCmUmAmCmA | 2051A | mAmGdTmAfAmGfUmUmCmCmC |
| 16 | 2 | mAmCmUmCmCmA | M16 | mA*mG*mG |
| BBD- | BBD- | mU*mG*mGmGmAmAfC | BBD- | VPU*dG*mGmAisoGNA- |
| 2051.2 | 2051SM | mUfUfAfCmUmAmCmA | 2051A | GmUmUmGmUmAmGdTmAfAmGf |
| 17 | 2 | mAmCmUmCmCmA | M17 | UmUmCmCmCmA*mG*mG |
| BBD- | BBD- | mU*mG*mGmGmAmAfC | BBD- | VPU*dG*mGmAmGisoGNA- |
| 2051.2 | 2051SM | mUfUfAfCmUmAmCmA | 2051A | TmUmGmUmAmGdTmAfAmGfUm |
| 18 | 2 | mAmCmUmCmCmA | M18 | UmCmCmCmA*mG*mG |
| BBD- | BBD- | mU*mG*mGmGmAmAfC | BBD- | VPU*dG*mGmAmGmUisoGNA- |
| 2051.2 | 2051SM | mUfUfAfCmUmAmCmA | 2051A | TmGmUmAmGdTmAfAmGfUmUm |
| 19 | 2 | mAmCmUmCmCmA | M19 | CmCmCmA*mG*mG |
| BBD- | BBD- | mU*mG*mGmGmAmAfC | BBD- | mU*fG*mGmAmGmUmUmGmUfA |
| 2051.3 | 2051SM | mUfUfAdCmUmAmCmA | 2051A | mGdTmAfAmGfUmUmCmCmCmA |
| 1 | 3 | mAmCmUmCmCmA | M1 | *mG*mG |
| BBD- | BBD- | mU*mG*mGmGmAmAfC | BBD- | mU*fG*mGmAisoGNA- |
| 2051.3 | 2051SM | mUfUfAdCmUmAmCmA | 2051A | GmUmUmGmUfAmGdTmAfAmGf |
| 5 | 3 | mAmCmUmCmCmA | M5 | UmUmCmCmCmA*mG*mG |
| BBD- | BBD- | mU*mG*mGmGmAmAfC | BBD- | mU*fG*mGmAmGisoGNA- |
| 2051.3 | 2051SM | mUfUfAdCmUmAmCmA | 2051A | TmUmGmUfAmGdTmAfAmGfUm |
| 6 | 3 | mAmCmUmCmCmA | M6 | UmCmCmCmA*mG*mG |
| BBD- | BBD- | mU*mG*mGmGmAmAfC | BBD- | mU*fG*mGmAmGmUisoGNA- |
| 2051.3 | 2051SM | mUfUfAdCmUmAmCmA | 2051A | TmGmUfAmGdTmAfAmGfUmUm |
| 7 | 3 | mAmCmUmCmCmA | M7 | CmCmCmA*mG*mG |
| BBD- | BBD- | mU*mG*mGmGmAmAfC | BBD- | VPU*fG*mGmAmGmUmUmGmUf |
| 2051.3 | 2051SM | mUfUfAdCmUmAmCmA | 2051A | AmGdTmAfAmGfUmUmCmCmCm |
| 8 | 3 | mAmCmUmCmCmA | M8 | A*mG*mG |
| BBD- | BBD- | mU*mG*mGmGmAmAfC | BBD- | VPU*fG*mGmAisoGNA- |
| 2051.3 | 2051SM | mUfUfAdCmUmAmCmA | 2051A | GmUmUmGmUfAmGdTmAfAmGf |
| 9 | 3 | mAmCmUmCmCmA | M9 | UmUmCmCmCmA*mG*mG |
| BBD- | BBD- | mU*mG*mGmGmAmAfC | BBD- | VPU*fG*mGmAmGisoGNA- |
| 2051.3 | 2051SM | mUfUfAdCmUmAmCmA | 2051A | TmUmGmUfAmGdTmAfAmGfUm |
| 10 | 3 | mAmCmUmCmCmA | M10 | UmCmCmCmA*mG*mG |
| BBD- | BBD- | mU*mG*mGmGmAmAfC | BBD- | VPU*fG*mGmAmGmUisoGNA- |
| 2051.3 | 2051SM | mUfUfAdCmUmAmCmA | 2051A | TmGmUfAmGdTmAfAmGfUmUm |
| 11 | 3 | mAmCmUmCmCmA | M11 | CmCmCmA*mG*mG |
| BBD- | BBD- | mU*mG*mGmGmAmAfC | BBD- | mU*dG*mGmAmGmUmUmGmUm |
| 2051.3 | 2051SM | mUfUfAdCmUmAmCmA | 2051A | AmGdTmAfAmGfUmUmCmCmCm |
| 12 | 3 | mAmCmUmCmCmA | M12 | A*mG*mG |
| BBD- | BBD- | mU*mG*mGmGmAmAfC | BBD- | mU*dG*mGmAisoGNA- |
| 2051.3 | 2051SM | mUfUfAdCmUmAmCmA | 2051A | GmUmUmGmUmAmGdTmAfAmGf |
| 13 | 3 | mAmCmUmCmCmA | M13 | UmUmCmCmCmA*mG*mG |
| BBD- | BBD- | mU*mG*mGmGmAmAfC | BBD- | mU*dG*mGmAmGisoGNA- |
| 2051.3 | 2051SM | mUfUfAdCmUmAmCmA | 2051A | TmUmGmUmAmGdTmAfAmGfUm |
| 14 | 3 | mAmCmUmCmCmA | M14 | UmCmCmCmA*mG*mG |
| BBD- | BBD- | mU*mG*mGmGmAmAfC | BBD- | mU*dG*mGmAmGmUisoGNA- |
| 2051.3 | 2051SM | mUfUfAdCmUmAmCmA | 2051A | TmGmUmAmGdTmAfAmGfUmUm |
| 15 | 3 | mAmCmUmCmCmA | M15 | CmCmCmA*mG*mG |
| BBD- | BBD- | mU*mG*mGmGmAmAfC | BBD- | VPU*dG*mGmAmGmUmUmGmU |
| 2051.3 | 2051SM | mUfUfAdCmUmAmCmA | 2051A | mAmGdTmAfAmGfUmUmCmCmC |
| 16 | 3 | mAmCmUmCmCmA | M16 | mA*mG*mG |
| BBD- | BBD- | mU*mG*mGmGmAmAfC | BBD- | VPU*dG*mGmAisoGNA- |
| 2051.3 | 2051SM | mUfUfAdCmUmAmCmA | 2051A | GmUmUmGmUmAmGdTmAfAmGf |
| 17 | 3 | mAmCmUmCmCmA | M17 | UmUmCmCmCmA*mG*mG |
| BBD- | BBD- | mU*mG*mGmGmAmAfC | BBD- | VPU*dG*mGmAmGisoGNA- |
| 2051.3 | 2051SM | mUfUfAdCmUmAmCmA | 2051A | TmUmGmUmAmGdTmAfAmGfUm |
| 18 | 3 | mAmCmUmCmCmA | M18 | UmCmCmCmA*mG*mG |
| BBD- | BBD- | mU*mG*mGmGmAmAfC | BBD- | VPU*dG*mGmAmGmUisoGNA- |
| 2051.3 | 2051SM | mUfUfAdCmUmAmCmA | 2051A | TmGmUmAmGdTmAfAmGfUmUm |
| 19 | 3 | mAmCmUmCmCmA | M19 | CmCmCmA*mG*mG |

Example 3: Integration of the hTMPRSS6 (human TMPRSS6) gene into a liver-targeted AAV8 expression vector, followed by production of the virus (VectorBuilder, AAV8LP), and subsequent infection of mice to generate transgenic mice with stabe hTMPRSS6 expression. Primary mouse hepatocyte extraction: Primary hepatocytes from mouse were isolated

using inferior vena cava perfusion, followed by collagenase digestion. The digested liver tissue was filtered through a tissue cell strainer (BIOLOGIX, 15-1070) to obtain viable and healthy primary mouse hepatocytes, which were resuspended in DMEM supplemented with 10% FBS and 1X penicillin-streptomycin. The cell density was determined using a Scepter automated cell counter (Millipore).

96-Well plate transfection: In vitro experiments were conducted using primary mouse hepatocytes. siRNA, Opti-MEM, and INTERFERin (Polyplus Transfection) were combined and incubated in a 96-well plate and incubated at room temperature for 10 minutes (for free uptake siRNA, INTERFERin was not required). Complete culture medium containing primary mouse hepatocytes was then added to each well. The 96-well plate was incubated at 37° C. in a 5% CO₂ incubator for 24 hours.

RNA extraction and reverse transcription from a 96-well plate: mRNA was extracted from cells in the 96-well plate using the Dynabeads mRNA DIRECT Kit (Ambion). The culture medium was aspirated, and the wells were rinsed once with DPBS. Then, 50-300 μL of cell lysis buffer was added to each well, followed by 20-100 μL of beads. The plate was placed on a shaker for thorough mixing. Next, the 96-well plate was placed on a magnetic stand, and the lysis buffer was aspirated. Each well was then washed with 50-300 μL of Wash Buffer A, mixed thoroughly, and placed on the magnetic stand to remove the buffer. The beads were then resuspended in Wash Buffer B, transferred to a new 96-well plate, and placed on the magnetic stand to remove the buffer. The beads were once again resuspended in Wash Buffer B and transferred to a 96-well PCR plate. Meanwhile, reverse transcription reagents were prepared. The 96-well PCR plate was placed on a magnetic stand, and the wash buffer was aspirated. Then, 20 μL of reverse transcription reagent was added to each well, and the plate was sealed with sealing film. The plate was incubated at 25° C. for 10 minutes on a PCR instrument, then at 37° C. for 2 hours, followed by 85° C. for 5 minutes, and finally cooled to 4° C. to complete the reverse transcription process.

Real-time fluorescent quantitative PCR (qPCR): After reverse transcription, the 96-well plate was placed on a magnetic stand until the beads were completely adsorbed to the bottom. The reverse transcription reagent was then aspirated, and the prepared qPCR reaction mixture was added to the 96-well PCR plate. The plate was sealed with a sealing film and subjected to qPCR analysis using the StepOnePlus Real-Time PCR System (Applied Biosystems). The ΔΔCt method was used to analyze the data, and the test was normalized using cells transfected with a 1 nM negative control sequence.

Table 11 describes the experimental results of multiple modified BBD-2051 double-stranded siRNA sequences delivered via GalNAc at a concentration of 1 nM in primary hepatocytes derived from hTMPRSS6-expressing mice.

| | |
|---|---|
| | hTMPRSS6 mRNA level in |
| | primary mouse hepatocytes (%) |
| siRNA | (relative to negative control cells) |

| Duplex | 1 nM avg | SD |
|---|---|---|
| BBD-2051.31-Galnac | 68.10 | 21.32 |
| BBD-2051.35-Galnac | 5.33 | 3.94 |
| BBD-2051.36-Galnac | 24.19 | 5.96 |
| BBD-2051.37-Galnac | 17.61 | 2.60 |
| BBD-2051.21-Galnac | 31.55 | 4.88 |
| BBD-2051.25-Galnac | 0.65 | 0.10 |
| BBD-2051.26-Galnac | 3.03 | 0.85 |

Table 12 describes the experimental results of modified BBD-2051 double-stranded siRNA sequences screened at 1 nM transfection concentration in Hep3B cells.

| | |
|---|---|
| | mRNA level in Hep3B cells (%) |
| siRNA | (relative to negative control cells) |

| Duplex | 1 nM avg | SD |
|---|---|---|
| BBD-2051.21-Galnac | 20.05 | 6.29 |
| BBD-2051.25-Galnac | 12.77 | 1.47 |
| BBD-2051.26-Galnac | 13.90 | 2.50 |
| BBD-2051.27-Galnac | 15.63 | 3.54 |
| BBD-2051.29-Galnac | 14.90 | 3.23 |
| BBD-2051.210-Galnac | 15.23 | 1.88 |
| BBD-2051.211-Galnac | 9.40 | 0.51 |

Table 13 describes the experimental results of modified BBD-2051 double-stranded siRNA sequences delivered via GalNAc and screened at various concentrations in primary hepatocytes derived from hTMPRSS6-expressing mice.

| | hTMPRSS6 mRNA level in primary | | | | | |
|---|---|---|---|---|---|---|
| | mouse hepatocytes (%) | | | | | |
| siRNA | (relative to negative control cells) | | | | | |
| Duplex | 10 nM avg | SD | 2 nM avg | SD | 0.4 nM avg | SD |
| BBD-2051.29-Galnac | 15.20 | 1.35 | 14.77 | 3.20 | 66.23 | 29.81 |
| BBD-2051.210-Galnac | 1.30 | 0.36 | 15.00 | 3.71 | 51.80 | 6.52 |
| BBD-2051.211-Galnac | 1.77 | 0.49 | 14.53 | 1.46 | 47.47 | 18.07 |
| BBD-2051.28-Galnac | 2.30 | 1.50 | 26.13 | 3.59 | 63.13 | 14.84 |

Table 14 describes multiple modified sense strand sequences of BBD-2083.

| modified | |
|---|---|
| sense | |
| strand ID | modified sense strand sequence 5′-3′ |
| AD-1554911-S | mU*mG*mCmUmAmCmUmCfUfGfGmUmAmUmUmUmCm |
| | CmUmAmU |
| BBD-2083SM1 | mU*mG*mCmUmAmCfUmCdTfGfGmUfAmUmUmUmCm |
| | CmUmAmA |
| BBD-2083SM2 | mU*mG*mCmUmAmCfUmCfUfGdGmUfAmUmUmUmCm |
| | CmUmAmA |
| BBD-2083SM3 | mU*mG*mCmUmAmCfUmCfUfGfGmUmAmUmUmUmCm |
| | CmUmAmA |
| BBD-2083SM4 | mU*mG*mCmUmAmCfUmCfUfGdGmUmAmUmUmUmCm |
| | CmUmAmA |
| BBD-2083SM5 | mU*mG*mCmUmAmCfUmCfUdGfGmUmAmUmUmUmCm |
| | CmUmAmA |
| BBD-2083SM6 | mU*mG*mCmUmAmCfUmCdTfGfGmUmAmUmUmUmCm |
| | CmUmAmA |

Table 15 describes multiple modified antisense strand sequences of BBD-2083.

| modified | |
|---|---|
| antisense | |
| strand | modified antisense strand |
| ID | sequence 5′-3′ |
| AD- | mA*dT*mAmGdGmAdAmAmUmAmCdCmAfGm |
| 1554911-A | AmGmUmAmGmCmA*mC*mC |
| BBD- | mU*fU*mAmGmGmAmAmAmUfAmCdCmAfGm |
| 2083AM1 | AfGmUmAmGmCmA*mC*mC |
| BBD- | mU*fU*mAfGfGfAmAfAmUmAmCmCmAfGm |
| 2083AM2 | AfGmUfAmGmCmA*mC*mC |
| BBD- | mU*fU*mAmGmGmAmAmAmUfAmCfCmAfGm |
| 2083AM3 | AfGmUmAmGmCmA*mC*mC |
| BBD- | mU*fU*mAmGmGmAmAmAmUfAmCmCmAfGm |
| 2083AM4 | AfGmUmAmGmCmA*mC*mC |
| BBD- | mU*fU*mAmGmGmAmAmAmUmAmCfCmAfGm |
| 2083AM5 | AfGmUmAmGmCmA*mC*mC |
| BBD- | mU*fU*mAmGmGmAmAmAmUfAfCmCmAfGm |
| 2083AM6 | AfGmUmAmGmCmA*mC*mC |
| BBD- | mU*fU*mAmGmGmAmAmAmUmAfCmCmAfGm |
| 2083AM7 | AfGmUmAmGmCmA*mC*mC |
| BBD- | mU*fU*mAmGmGmAmAmAmUfAdCmCmAfGm |
| 2083AM8 | AfGmUmAmGmCmA*mC*mC |
| BBD- | mU*fU*mAmGmGmAmAmAmUfAmCmCdAfGm |
| 2083AM9 | AfGmUmAmGmCmA*mC*mC |
| BBD- | mU*fU*mAmGisoGNA- |
| 2083AM10 | GmAmAmAmUfAmCdCmAfGmAfGmUmAmGm |
| | CmA*mC*mC |
| BBD- | mU*fU*mAmGmGisoGNA- |
| 2083AM11 | AmAmAmUfAmCdCmAfGmAfGmUmAmGmCm |
| | A*mC*mC |
| BBD- | mU*fU*mAmGmGmAisoGNA- |
| 2083AM12 | AmAmUfAmCdCmAfGmAfGmUmAmGmCm |
| | A*mC*mC |
| BBD- | VPU*fU*mAmGmGmAmAmAmUfAmCdCmA |
| 2083AM13 | fGmAfGmUmAmGmCmA*mC*mC |
| BBD- | VPU*fU*mAmGisoGNA- |
| 2083AM14 | GmAmAmAmUfAmCdCmAfGmAfGmUmAmGm |
| | CmA*mC*mC |
| BBD- | VPU*fU*mAmGmGisoGNA- |
| 2083AM15 | AmAmAmUfAmCdCmAfGmAfGmUmAmGmCm |
| | A*mC*mC |
| BBD- | VPU*fU*mAmGmGmAisoGNA- |
| 2083AM16 | AmAmUfAmCdCmAfGmAfGmUmAmGmCmA* |
| | mC*mC |
| BBD- | mU*dT*mAmGmGmAmAmAmUmAmCdCmAfGm |
| 2083AM17 | AfGmUmAmGmCmA*mC*mC |
| BBD- | mU*dT*mAmGisoGNA- |
| 2083AM18 | GmAmAmAmUmAmCdCmAfGmAfGmUmAmGmC |
| | mA*mC*mC |
| BBD- | mU*dT*mAmGmGisoGNA- |
| 2083AM19 | AmAmAmUmAmCdCmAfGmAfGmUmAmGmCm |
| | A*mC*mC |
| BBD- | mU*dT*mAmGmGmAisoGNA- |
| 2083AM20 | AmAmUmAmCdCmAfGmAfGmUmAmGmCmA* |
| | mC*mC |
| BBD- | VPU*dT*mAmGmGmAmAmAmUmAmCdCmAf |
| 2083AM21 | GmAfGmUmAmGmCmA*mC*mC |
| BBD- | VPU*dT*mAmGisoGNA- |
| 2083AM22 | GmAmAmAmUmAmCdCmAfGmAfGmUmAmGm |
| | CmA*mC*mC |
| BBD- | VPU*dT*mAmGmGisoGNA- |
| 2083AM23 | AmAmAmUmAmCdCmAfGmAfGmUmAmGmCm |
| | A*mC*mC |
| BBD- | VPU*dT*mAmGmGmAisoGNA- |
| 2083AM24 | AmAmUmAmCdCmAfGmAfGmUmAmGmCmA |
| | *mC*mC |

Table 16 describes multiple modified double-stranded siRNA sequences of BBD-2083.

| | | | modified | |
|---|---|---|---|---|
| | modified | | anti- | |
| | sense | modified sense | sense | modified antisense |
| duplex | strand | strand sequence | strand | strand sequence |
| ID | ID | 5′-3′ | ID | 5′-3′ |
| AD- | AD- | mU*mG*mCmUmAmCmU | AD- | mA*dT*mAmGdGmAdAmAmUm |
| 15549 | 15549 | mCfUfGfGmUmAmUmUm | 155491 | AmCdCmAfGmAmGmUmAmGm |
| 11 | 11-S | UmCmCmUmAmU | 1-A | CmA*mC*mC |
| BBD- | BBD- | mU*mG*mCmUmAmCfU | BBD- | mU*fU*mAmGmGmAmAmAmUf |
| 2083. | 2083S | mCfUfGdGmUfAmUmUm | 2083A | AmCdCmAfGmAfGmUmAmGmC |
| 21 | M2 | UmCmCmUmAmA | M1 | mA*mC*mC |
| BBD- | BBD- | mU*mG*mCmUmAmCfU | BBD- | mU*fU*mAmGmGmAmAmAmUf |
| 2083. | 2083S | mCfUfGfGmUmAmUmUm | 2083A | AmCdCmAfGmAfGmUmAmGmC |
| 31 | M3 | UmCmCmUmAmA | M1 | mA*mC*mC |
| BBD- | BBD- | mU*mG*mCmUmAmCfU | BBD- | mU*fU*mAmGmGmAmAmAmUf |
| 2083. | 2083S | mCfUfGfGmUmAmUmUm | 2083A | AmCfCmAfGmAfGmUmAmGmC |
| 33 | M3 | UmCmCmUmAmA | M3 | mA*mC*mC |
| BBD- | BBD- | mU*mG*mCmUmAmCfU | BBD- | mU*fU*mAmGmGmAmAmAmUf |
| 2083. | 2083S | mCfUfGdGmUmAmUmUm | 2083A | AmCfCmAfGmAfGmUmAmGmC |
| 43 | M4 | UmCmCmUmAmA | M3 | mA*mC*mC |
| BBD- | BBD- | mU*mG*mCmUmAmCfU | BBD- | mU*fU*mAmGmGmAmAmAmUf |
| 2083. | 2083S | mCfUfGfGmUmAmUmUm | 2083A | AmCmCmAfGmAfGmUmAmGm |
| 34 | M3 | UmCmCmUmAmA | M4 | CmA*mC*mC |
| BBD- | BBD- | mU*mG*mCmUmAmCfU | BBD- | mU*fU*mAmGmGmAmAmAmU |
| 2083. | 2083S | mCfUfGfGmUmAmUmUm | 2083A | mAmCfCmAfGmAfGmUmAmGm |
| 35 | M3 | UmCmCmUmAmA | M5 | CmA*mC*mC |
| BBD- | BBD- | mU*mG*mCmUmAmCfU | BBD- | mU*fU*mAmGmGmAmAmAmUf |
| 2083. | 2083S | mCfUfGfGmUmAmUmUm | 2083A | AfCmCmAfGmAfGmUmAmGmC |
| 36 | M3 | UmCmCmUmAmA | M6 | mA*mC*mC |
| BBD- | BBD- | mU*mG*mCmUmAmCfU | BBD- | mU*fU*mAmGmGmAmAmAmU |
| 2083. | 2083S | mCfUfGfGmUmAmUmUm | 2083A | mAfCmCmAfGmAfGmUmAmGm |
| 37 | M3 | UmCmCmUmAmA | M7 | CmA*mC*mC |
| BBD- | BBD- | mU*mG*mCmUmAmCfU | BBD- | mU*fU*mAmGmGmAmAmAmUf |
| 2083. | 2083S | mCfUfGfGmUmAmUmUm | 2083A | AdCmCmAfGmAfGmUmAmGmC |
| 38 | M3 | UmCmCmUmAmA | M8 | mA*mC*mC |
| BBD- | BBD- | mU*mG*mCmUmAmCfU | BBD- | mU*fU*mAmGmGmAmAmAmUf |
| 2083. | 2083S | mCfUfGfGmUmAmUmUm | 2083A | AmCmCdAfGmAfGmUmAmGmC |
| 39 | M3 | UmCmCmUmAmA | M9 | mA*mC*mC |
| BBD- | BBD- | mU*mG*mCmUmAmCfU | BBD- | mU*fU*mAmGmGmAmAmAmUf |
| 2083. | 2083S | mCfUfGdGmUmAmUmUm | 2083A | AmCmCmAfGmAfGmUmAmGm |
| 44 | M4 | UmCmCmUmAmA | M4 | CmA*mC*mC |
| BBD- | BBD- | mU*mG*mCmUmAmCfU | BBD- | mU*fU*mAmGmGmAmAmAmUf |
| 2083. | 2083S | mCfUdGfGmUmAmUmUm | 2083A | AmCmCmAfGmAfGmUmAmGm |
| 54 | M5 | UmCmCmUmAmA | M4 | CmA*mC*mC |
| BBD- | BBD- | mU*mG*mCmUmAmCfU | BBD- | mU*fU*mAmGmGmAmAmAmUf |
| 2083. | 2083S | mCdTfGfGmUmAmUmUm | 2083A | AmCmCmAfGmAfGmUmAmGm |
| 64 | M6 | UmCmCmUmAmA | M4 | CmA*mC*mC |
| BBD- | BBD- | mU*mG*mCmUmAmCfU | BBD- | mU*fU*mAmGisoGNA- |
| 2083. | 2083S | mCfUfGfGmUmAmUmUm | 2083A | GmAmAmAmUfAmCdCmAfGm |
| 310 | M3 | UmCmCmUmAmA | M10 | AfGmUmAmGmCmA*mC*mC |
| BBD- | BBD- | mU*mG*mCmUmAmCfU | BBD- | mU*fU*mAmGmGisoGNA- |
| 2083. | 2083S | mCfUfGfGmUmAmUmUm | 2083A | AmAmAmUfAmCdCmAfGmAfG |
| 311 | M3 | UmCmCmUmAmA | M11 | mUmAmGmCmA*mC*mC |
| BBD- | BBD- | mU*mG*mCmUmAmCfU | BBD- | mU*fU*mAmGmGmAisoGNA- |
| 2083. | 2083S | mCfUfGfGmUmAmUmUm | 2083A | AmAmUfAmCdCmAfGmAfGmU |
| 312 | M3 | UmCmCmUmAmA | M12 | mAmGmCmA*mC*mC |
| BBD- | BBD- | mU*mG*mCmUmAmCfU | BBD- | VPU*fU*mAmGmGmAmAmAm |
| 2083. | 2083S | mCfUfGfGmUmAmUmUm | 2083A | UfAmCdCmAfGmAfGmUmAmG |
| 313 | M3 | UmCmCmUmAmA | M13 | mCmA*mC*mC |
| BBD- | BBD- | mU*mG*mCmUmAmCfU | BBD- | VPU*fU*mAmGisoGNA- |
| 2083. | 2083S | mCfUfGfGmUmAmUmUm | 2083A | GmAmAmAmUfAmCdCmAfGm |
| 314 | M3 | UmCmCmUmAmA | M14 | AfGmUmAmGmCmA*mC*mC |
| BBD- | BBD- | mU*mG*mCmUmAmCfU | BBD- | VPU*fU*mAmGmGisoGNA- |
| 2083. | 2083S | mCfUfGfGmUmAmUmUm | 2083A | AmAmAmUfAmCdCmAfGmAfG |
| 315 | M3 | UmCmCmUmAmA | M15 | mUmAmGmCmA*mC*mC |
| BBD- | BBD- | mU*mG*mCmUmAmCfU | BBD- | VPU*fU*mAmGmGmAisoGNA- |
| 2083. | 2083S | mCfUfGfGmUmAmUmUm | 2083A | AmAmUfAmCdCmAfGmAfGmU |
| 316 | M3 | UmCmCmUmAmA | M16 | mAmGmCmA*mC*mC |
| BBD- | BBD- | mU*mG*mCmUmAmCfU | BBD- | mU*dT*mAmGmGmAmAmAmU |
| 2083. | 2083S | mCfUfGfGmUmAmUmUm | 2083A | mAmCdCmAfGmAfGmUmAmG |
| 317 | M3 | UmCmCmUmAmA | M17 | mCmA*mC*mC |
| BBD- | BBD- | mU*mG*mCmUmAmCfU | BBD- | mU*dT*mAmGisoGNA- |
| 2083. | 2083S | mCfUfGfGmUmAmUmUm | 2083A | GmAmAmAmUmAmCdCmAfGm |
| 318 | M3 | UmCmCmUmAmA | M18 | AfGmUmAmGmCmA*mC*mC |
| BBD- | BBD- | mU*mG*mCmUmAmCfU | BBD- | mU*dT*mAmGmGisoGNA- |
| 2083. | 2083S | mCfUfGfGmUmAmUmUm | 2083A | AmAmAmUmAmCdCmAfGmAf |
| 319 | M3 | UmCmCmUmAmA | M19 | GmUmAmGmCmA*mC*mC |
| BBD- | BBD- | mU*mG*mCmUmAmCfU | BBD- | mU*dT*mAmGmGmAisoGNA- |
| 2083. | 2083S | mCfUfGfGmUmAmUmUm | 2083A | AmAmUmAmCdCmAfGmAfGm |
| 320 | M3 | UmCmCmUmAmA | M20 | UmAmGmCmA*mC*mC |
| BBD- | BBD- | mU*mG*mCmUmAmCfU | BBD- | VPU*dT*mAmGmGmAmAmAm |
| 2083. | 2083S | mCfUfGfGmUmAmUmUm | 2083A | UmAmCdCmAfGmAfGmUmAm |
| 321 | M3 | UmCmCmUmAmA | M21 | GmCmA*mC*mC |
| BBD- | BBD- | mU*mG*mCmUmAmCfU | BBD- | VPU*dT*mAmGisoGNA- |
| 2083. | 2083S | mCfUfGfGmUmAmUmUm | 2083A | GmAmAmAmUmAmCdCmAfGm |
| 322 | M3 | UmCmCmUmAmA | M22 | AfGmUmAmGmCmA*mC*mC |
| BBD- | BBD- | mU*mG*mCmUmAmCfU | BBD- | VPU*dT*mAmGmGisoGNA- |
| 2083. | 2083S | mCfUfGfGmUmAmUmUm | 2083A | AmAmAmUmAmCdCmAfGmAf |
| 323 | M3 | UmCmCmUmAmA | M23 | GmUmAmGmCmA*mC*mC |
| BBD- | BBD- | mU*mG*mCmUmAmCfU | BBD- | VPU*dT*mAmGmGmAisoGNA- |
| 2083. | 2083S | mCfUfGfGmUmAmUmUm | 2083A | AmAmUmAmCdCmAfGmAfGm |
| 324 | M3 | UmCmCmUmAmA | M24 | UmAmGmCmA*mC*mC |
| BBD- | BBD- | mU*mG*mCmUmAmCfU | BBD- | mU*fU*mAmGmGmAmAmAmUf |
| 2083. | 2083S | mCfUfGdGmUmAmUmUm | 2083A | AmCdCmAfGmAfGmUmAmGmC |
| 41 | M4 | UmCmCmUmAmA | M1 | mA*mC*mC |
| BBD- | BBD- | mU*mG*mCmUmAmCfU | BBD- | mU*fU*mAmGisoGNA- |
| 2083. | 2083S | mCfUfGdGmUmAmUmUm | 2083A | GmAmAmAmUfAmCdCmAfGm |
| 410 | M4 | UmCmCmUmAmA | M10 | AfGmUmAmGmCmA*mC*mC |
| BBD- | BBD- | mU*mG*mCmUmAmCfU | BBD- | mU*fU*mAmGmGisoGNA- |
| 2083. | 2083S | mCfUfGdGmUmAmUmUm | 2083A | AmAmAmUfAmCdCmAfGmAfG |
| 411 | M4 | UmCmCmUmAmA | M11 | mUmAmGmCmA*mC*mC |
| BBD- | BBD- | mU*mG*mCmUmAmCfU | BBD- | mU*fU*mAmGmGmAisoGNA- |
| 2083. | 2083S | mCfUfGdGmUmAmUmUm | 2083A | AmAmUfAmCdCmAfGmAfGmU |
| 412 | M4 | UmCmCmUmAmA | M12 | mAmGmCmA*mC*mC |
| BBD- | BBD- | mU*mG*mCmUmAmCfU | BBD- | VPU*fU*mAmGmGmAmAmAm |
| 2083. | 2083S | mCfUfGdGmUmAmUmUm | 2083A | UfAmCdCmAfGmAfGmUmAmG |
| 413 | M4 | UmCmCmUmAmA | M13 | mCmA*mC*mC |
| BBD- | BBD- | mU*mG*mCmUmAmCfU | BBD- | VPU*fU*mAmGisoGNA- |
| 2083. | 2083S | mCfUfGdGmUmAmUmUm | 2083A | GmAmAmAmUfAmCdCmAfGm |
| 414 | M4 | UmCmCmUmAmA | M14 | AfGmUmAmGmCmA*mC*mC |
| BBD- | BBD- | mU*mG*mCmUmAmCfU | BBD- | VPU*fU*mAmGmGisoGNA- |
| 2083. | 2083S | mCfUfGdGmUmAmUmUm | 2083A | AmAmAmUfAmCdCmAfGmAfG |
| 415 | M4 | UmCmCmUmAmA | M15 | mUmAmGmCmA*mC*mC |
| BBD- | BBD- | mU*mG*mCmUmAmCfU | BBD- | VPU*fU*mAmGmGmAisoGNA- |
| 2083. | 2083S | mCfUfGdGmUmAmUmUm | 2083A | AmAmUfAmCdCmAfGmAfGmU |
| 416 | M4 | UmCmCmUmAmA | M16 | mAmGmCmA*mC*mC |
| BBD- | BBD- | mU*mG*mCmUmAmCfU | BBD- | mU*dT*mAmGmGmAmAmAmU |
| 2083. | 2083S | mCfUfGdGmUmAmUmUm | 2083A | mAmCdCmAfGmAfGmUmAmG |
| 417 | M4 | UmCmCmUmAmA | M17 | mCmA*mC*mC |
| BBD- | BBD- | mU*mG*mCmUmAmCfU | BBD- | mU*dT*mAmGisoGNA- |
| 2083. | 2083S | mCfUfGdGmUmAmUmUm | 2083A | GmAmAmAmUmAmCdCmAfGm |
| 418 | M4 | UmCmCmUmAmA | M18 | AfGmUmAmGmCmA*mC*mC |
| BBD- | BBD- | mU*mG*mCmUmAmCfU | BBD- | mU*dT*mAmGmGisoGNA- |
| 2083. | 2083S | mCfUfGdGmUmAmUmUm | 2083A | AmAmAmUmAmCdCmAfGmAf |
| 419 | M4 | UmCmCmUmAmA | M19 | GmUmAmGmCmA*mC*mC |
| BBD- | BBD- | mU*mG*mCmUmAmCfU | BBD- | mU*dT*mAmGmGmAisoGNA- |
| 2083. | 2083S | mCfUfGdGmUmAmUmUm | 2083A | AmAmUmAmCdCmAfGmAfGm |
| 420 | M4 | UmCmCmUmAmA | M20 | UmAmGmCmA*mC*mC |
| BBD- | BBD- | mU*mG*mCmUmAmCfU | BBD- | VPU*dT*mAmGmGmAmAmAm |
| 2083. | 2083S | mCfUfGdGmUmAmUmUm | 2083A | UmAmCdCmAfGmAfGmUmAm |
| 421 | M4 | UmCmCmUmAmA | M21 | GmCmA*mC*mC |
| BBD- | BBD- | mU*mG*mCmUmAmCfU | BBD- | VPU*dT*mAmGisoGNA- |
| 2083. | 2083S | mCfUfGdGmUmAmUmUm | 2083A | GmAmAmAmUmAmCdCmAfGm |
| 422 | M4 | UmCmCmUmAmA | M22 | AfGmUmAmGmCmA*mC*mC |
| BBD- | BBD- | mU*mG*mCmUmAmCfU | BBD- | VPU*dT*mAmGmGisoGNA- |
| 2083. | 2083S | mCfUfGdGmUmAmUmUm | 2083A | AmAmAmUmAmCdCmAfGmAf |
| 423 | M4 | UmCmCmUmAmA | M23 | GmUmAmGmCmA*mC*mC |
| BBD- | BBD- | mU*mG*mCmUmAmCfU | BBD- | VPU*dT*mAmGmGmAisoGNA- |
| 2083. | 2083S | mCfUfGdGmUmAmUmUm | 2083A | AmAmUmAmCdCmAfGmAfGm |
| 424 | M4 | UmCmCmUmAmA | M24 | UmAmGmCmA*mC*mC |
| AD- | AD- | mU*mG*mCmUmAmCmU | AD- | mA*dT*mAmGdGmAdAmAmUm |
| 15549 | 15549 | mCfUfGfGmUmAmUmUm | 155491 | AmCdCmAfGmAmGmUmAmGm |
| 11 | 11-S | UmCmCmUmAmU | 1-A | CmA*mC*mC |

Table 17 describes the experimental results of multiple modified BBD-2083 double-stranded siRNA sequences and AD-1554911 delivered via GalNAc at a concentration of 1 nM in primary hepatocytes derived from hTMPRSS6-expressing mice.

| | hTMPRSS6 mRNA level in primary mouse | |
|---|---|---|
| | hepatocytes (%) | |
| | (relative to negative control cells) | |
| siRNA Duplex | 1 nM avg | SD |
| TMPRSS6-hcm-9- | 27.69 | 7.00 |
| Galnac | | |
| BBD-2083.21-Galnac | 19.61 | 6.00 |
| BBD-2083.31-Galnac | 8.71 | 1.13 |
| BBD-2083.33-Galnac | 13.36 | 6.68 |
| BBD-2083.43-Galnac | 19.13 | 8.74 |
| BBD-2083.34-Galnac | 13.53 | 7.78 |
| BBD-2083.44-Galnac | 21.65 | 3.11 |
| BBD-2083.35-Galnac | 13.42 | 6.68 |
| BBD-2083.36-Galnac | 8.63 | 2.61 |
| BBD-2083.37-Galnac | 15.09 | 0.41 |
| BBD-2083.38-Galnac | 38.70 | 10.53 |
| BBD-2083.39-Galnac | 12.75 | 2.51 |
| BBD-2083.54-Galnac | 28.72 | 8.42 |
| BBD-2083.64-Galnac | 30.53 | 7.52 |
| BBD-2083.310-Galnac | 5.95 | 2.20 |
| BBD-2083.311-Galnac | 6.37 | 0.99 |
| BBD-2083.312-Galnac | 14.20 | 3.37 |
| BBD-2083.313-Galnac | 0.77 | 0.16 |
| BBD-2083.41-Galnac | 4.48 | 1.46 |
| BBD-2083.410-Galnac | 7.70 | 1.50 |
| BBD-2083.411-Galnac | 13.35 | 11.73 |
| BBD-2083.412-Galnac | 28.42 | 17.90 |
| BBD-2083.413-Galnac | 0.98 | 0.53 |
| AD-1554911-Galnac | 14.60 | 5.68 |

Table 18 describes the experimental results of modified BBD-2083 double-stranded siRNA sequences and AD-1554911 screened at 1 nM transfection concentration in Hep3B cells.

| | |
|---|---|
| | mRNA level in Hep3B cells (%) |
| siRNA | (relative to negative control cells) |

| Duplex | 1 nM avg | SD |
|---|---|---|
| BBD-2083.31-Galnac | 17.27 | 2.21 |
| BBD-2083.38-Galnac | 46.75 | 7.71 |
| BBD-2083.39-Galnac | 45.10 | 5.65 |
| BBD-2083.34-Galnac | 40.95 | 3.94 |
| BBD-2083.44-Galnac | 47.13 | 5.55 |
| BBD-2083.54-Galnac | 53.17 | 9.15 |
| BBD-2083.64-Galnac | 37.84 | 3.27 |
| BBD-2083.310-Galnac | 8.66 | 0.23 |
| BBD-2083.311-Galnac | 13.47 | 3.50 |
| BBD-2083.312-Galnac | 11.08 | 0.50 |
| BBD-2083.41-Galnac | 14.94 | 7.68 |
| AD-1554911-Galnac | 19.46 | 8.51 |

Table 19 describes the experimental results of modified BBD-2083 double-stranded siRNA sequences and AD-1554911 screened at 0.3 nM transfection concentration in Hep3B cells

mRNA level in Hep3B cells (%)
(relative to negative control cells)

| siRNA Duplex | 0.3 nM avg | SD |
|---|---|---|
| BBD-2083.313-Galnac | 33.48 | 7.71 |
| BBD-2083.413-Galnac | 42.68 | 10.02 |
| BBD-2083.414-Galnac | 43.21 | 11.53 |
| BBD-2083.415-Galnac | 52.25 | 13.02 |
| AD-1554911-Galnac | 59.07 | 4.07 |

Through the above experiments, we found that the activity of the 2083 sequence was significantly enhanced in vitro after the addition of VP modifications.

Table 20 describes multiple modified sense strand sequences of BBD-2047.

| modified | |
|---|---|
| sense | |
| strand | modified sense strand |
| ID | sequence 5′-3′ |
| BBD- | mC*mG*mCmCmUmGfGmGdAfAfCmUfUm |
| 2047SM1 | AmCmUmAmCmAmAmU |
| BBD- | mC*mG*mCmCmUmGfGmGfAfAdCmUfUm |
| 2047SM2 | AmCmUmAmCmAmAmU |
| BBD- | mC*fG*mCfCmUfGmGfGmAfAmCfUmUf |
| 2047SM3 | AmCfUmAfCmAfAmU |
| BBD- | mC*mG*mCmCmUmGfGmGfAfAfCmUfUm |
| 2047SM4 | AmCmUmAmCmAmAmU |
| BBD- | mC*mG*mCmCmUmGfGmGfAfAdCmUfUm |
| 2047SM5 | AmCmUmAmCmAmAmU |
| BBD- | mC*mG*mCmCmUmGfGmGfAmAdCmUfUm |
| 2047SM6 | AmCmUmAmCmAmAmU |
| BBD- | mC*mG*mCmCmUmGfGmGfAfAdCmUmUm |
| 2047SM7 | AmCmUmAmCmAmAmU |
| BBD- | mC*mG*mCmCmUmGfGmGfAfAfCmUmUm |
| 2047SM8 | AmCmUmAmCmAmAmU |
| BBD- | mC*mG*mCmCmUmGfGmGfAdAfCmUmUm |
| 2047SM9 | AmCmUmAmCmAmAmU |
| BBD- | mC*mG*mCmCmUmGfGmGdAfAfCmUmUm |
| 2047SM10 | AmCmUmAmCmAmAmU |

Table 21 describes multiple modified antisense strand sequences of BBD-2047.

| modified | |
|---|---|
| antisense | modified antisense strand |
| strand ID | sequence 5′-3′ |
| BBD- | mA*fU*mUmGmUmAmGmUmAfAmGdTmUf |
| 2047AM1 | CmCfCmAmGmGmCmG*mG*mG |
| BBD- | mA*fU*mUfGfUfAmGfUmAmAmGmUmUf |
| 2047AM2 | CmCfCmAfGmGmCmG*mG*mG |
| BBD- | fA*mU*fUmGfUmAfGmUmAfAmGdTmUf |
| 2047AM3 | CmCfCmAmGmGmCmG*mG*mG |
| BBD- | mA*fU*mUmGfUmAmGmUmAfAmGdTmUf |
| 2047AM4 | CmCfCmAmGmGmCmG*mG*mG |
| BBD- | fA*mU*fUmGfUmAfGmUmAfAmGdTmUf |
| 2047AM5 | CmCfCmAfGmGmCmG*mG*mG |
| BBD- | fA*mU*fUmGfUmAfGmUfAmAmGdTmUf |
| 2047AM6 | CmCfCmAfGmGmCmG*mG*mG |
| BBD- | fA*mU*fUmGfUmAfGmUfAmAdGmUmUf |
| 2047AM7 | CmCfCmAfGmGmCmG*mG*mG |
| BBD- | fA*mU*fUmGfUmAfGmUfAmAdGmUmUf |
| 2047AM8 | CmCfCmAfGmGfCmG*fG*mG |
| BBD- | fA*mU*fUmGfUmAfGmUmAfAmGmUmUf |
| 2047AM9 | CmCfCmAmGmGmCmG*mG*mG |
| BBD- | fA*mU*fUmGfUmAfGmUmAmAmGmUmUf |
| 2047AM1 | CmCfCmAmGmGmCmG*mG*mG |
| 0 | |
| BBD- | mA*fU*mUfGfUfAmGfUmAmAmGdTmUf |
| 2047AM1 | CmCfCmAfGmGmCmG*mG*mG |
| 1 | |
| BBD- | mA*fU*mUfGfUfAmGfUmAmAdGmUmUf |
| 2047AM1 | CmCfCmAfGmGmCmG*mG*mG |
| 2 | |
| BBD- | mA*fU*mUfGfUfAmGfUmAfAmGdTmUf |
| 2047AM1 | CmCfCmAfGmGmCmG*mG*mG |
| 3 | |
| BBD- | mA*fU*mUfGfUfAmGfUmAmAmGfUmUf |
| 2047AM1 | CmCfCmAfGmGmCmG*mG*mG |
| 4 | |
| BBD- | mA*fU*mUfGfUfAmGfUmAmAfGmUmUf |
| 2047AM1 | CmCfCmAfGmGmCmG*mG*mG |
| 5 | |
| BBD- | mA*fU*mUfGfUfAmGfUmAfAmGfUmUf |
| 2047AM1 | CmCfCmAfGmGmCmG*mG*mG |
| 6 | |
| BBD- | mA*fU*mUfGfUfAmGfUmAfAfGmUmUf |
| 2047AM1 | CmCfCmAfGmGmCmG*mG*mG |
| 7 | |
| BBD- | mA*fU*mUfGfUfAmGfUmAfAmGmUmUf |
| 2047AM1 | CmCfCmAfGmGmCmG*mG*mG |
| 8 | |
| BBD- | mA*fU*mUfGfUfAmGfUfAmAmGmUmUf |
| 2047AM1 | CmCfCmAfGmGmCmG*mG*mG |
| 9 | |
| BBD- | mA*fU*mUfGfUfAmGfUmAmAmGmUmUf |
| 2047AM2 | CmCfCmAmGmGmCmG*mG*mG |
| 0 | |
| BBD- | mA*fU*mUfGfUfAmGfUmAmAmGmUmUf |
| 2047AM2 | CmCfCmAfGfGmCmG*mG*mG |
| 1 | |
| BBD- | mA*fU*mUfGfUfAmGfUmAfAmGmUmUf |
| 2047AM2 | CmCfCmAmGmGmCmG*mG*mG |
| 2 | |
| BBD- | mA*fU*mUmGmUmAmGmUmAfAmGmUmUf |
| 2047AM2 | CmCfCmAmGmGmCmG*mG*mG |
| 3 | |
| BBD- | mA*fU*mUmGmUmAmGmUmAfAmGfUmUf |
| 2047AM2 | CmCfCmAmGmGmCmG*mG*mG |
| 4 | |
| BBD- | mA*fU*mUmGmUmAmGmUmAfAdGmUmUf |
| 2047AM2 | CmCfCmAmGmGmCmG*mG*mG |
| 5 | |
| BBD- | mA*fU*mUmGmUmAmGmUmAfAmGmUdTf |
| 2047AM2 | CmCfCmAmGmGmCmG*mG*mG |
| 6 | |

Table 22 describes multiple modified double-stranded siRNA sequences of BBD-2047.

| | modified | modified | modified | modified |
|---|---|---|---|---|
| | sense | sense strand | antisense | antisense strand |
| duplex ID | strand ID | sequence 5′-3′ | strand ID | sequence 5′-3′ |
| BBD-2047.14 | BBD-2047SM1 | mC*mG*mCmC | BBD-2047AM4 | mA*fU*mUmGf |
| | | mUmGfGmGdAf | | UmAmGmUmAf |
| | | AfCmUfUmAm | | AmGdTmUfCm |
| | | CmUmAmCmA | | CfCmAmGmGm |
| | | mAmU | | CmG*mG*mG |
| BBD-2047.13 | BBD-2047SM1 | mC*mG*mCmC | BBD-2047AM3 | fA*mU*fUmGfU |
| | | mUmGfGmGdAf | | mAfGmUmAfA |
| | | AfCmUfUmAm | | mGdTmUfCmCf |
| | | CmUmAmCmA | | CmAmGmGmC |
| | | mAmU | | mG*mG*mG |
| BBD-2047.12 | BBD-2047SM1 | mC*mG*mCmC | BBD-2047AM2 | mA*fU*mUfGfU |
| | | mUmGfGmGdAf | | fAmGfUmAmA |
| | | AfCmUfUmAm | | mGmUmUfCmC |
| | | CmUmAmCmA | | fCmAfGmGmC |
| | | mAmU | | mG*mG*mG |
| BBD-2047.42 | BBD-2047SM4 | mC*mG*mCmC | BBD-2047AM2 | mA*fU*mUfGfU |
| | | mUmGfGmGfAf | | fAmGfUmAmA |
| | | AfCmUfUmAm | | mGmUmUfCmC |
| | | CmUmAmCmA | | fCmAfGmGmC |
| | | mAmU | | mG*mG*mG |
| BBD-2047.43 | BBD-2047SM4 | mC*mG*mCmC | BBD-2047AM3 | fA*mU*fUmGfU |
| | | mUmGfGmGfAf | | mAfGmUmAfA |
| | | AfCmUfUmAm | | mGdTmUfCmCf |
| | | CmUmAmCmA | | CmAmGmGmC |
| | | mAmU | | mG*mG*mG |
| BBD-2047.53 | BBD-2047SM5 | mC*mG*mCmC | BBD-2047AM3 | fA*mU*fUmGfU |
| | | mUmGfGmGfAf | | mAfGmUmAfA |
| | | AdCmUfUmAm | | mGdTmUfCmCf |
| | | CmUmAmCmA | | CmAmGmGmC |
| | | mAmU | | mG*mG*mG |
| BBD-2047.55 | BBD-2047SM5 | mC*mG*mCmC | BBD-2047AM5 | fA*mU*fUmGfU |
| | | mUmGfGmGfAf | | mAfGmUmAfA |
| | | AdCmUfUmAm | | mGdTmUfCmCf |
| | | CmUmAmCmA | | CmAfGmGmCm |
| | | mAmU | | G*mG*mG |
| BBD-2047.56 | BBD-2047SM5 | mC*mG*mCmC | BBD-2047AM6 | fA*mU*fUmGfU |
| | | mUmGfGmGfAf | | mAfGmUfAmA |
| | | AdCmUfUmAm | | mGdTmUfCmCf |
| | | CmUmAmCmA | | CmAfGmGmCm |
| | | mAmU | | G*mG*mG |
| BBD-2047.57 | BBD-2047SM5 | mC*mG*mCmC | BBD-2047AM7 | fA*mU*fUmGfU |
| | | mUmGfGmGfAf | | mAfGmUfAmAd |
| | | AdCmUfUmAm | | GmUmUfCmCfC |
| | | CmUmAmCmA | | mAfGmGmCmG |
| | | mAmU | | *mG*mG |
| BBD-2047.58 | BBD-2047SM5 | mC*mG*mCmC | BBD-2047AM8 | fA*mU*fUmGfU |
| | | mUmGfGmGfAf | | mAfGmUfAmAd |
| | | AdCmUfUmAm | | GmUmUfCmCfC |
| | | CmUmAmCmA | | mAfGmGfCmG* |
| | | mAmU | | fG*mG |
| BBD-2047.59 | BBD-2047SM5 | mC*mG*mCmC | BBD-2047AM9 | fA*mU*fUmGfU |
| | | mUmGfGmGfAf | | mAfGmUmAfA |
| | | AdCmUfUmAm | | mGmUmUfCmC |
| | | CmUmAmCmA | | fCmAmGmGmC |
| | | mAmU | | mG*mG*mG |
| BBD-2047.510 | BBD-2047SM5 | mC*mG*mCmC | BBD-2047AM10 | fA*mU*fUmGfU |
| | | mUmGfGmGfAf | | mAfGmUmAmA |
| | | AdCmUfUmAm | | mGmUmUfCmC |
| | | CmUmAmCmA | | fCmAmGmGmC |
| | | mAmU | | mG*mG*mG |
| BBD-2047.511 | BBD-2047SM5 | mC*mG*mCmC | BBD-2047AM11 | mA*fU*mUfGfU |
| | | mUmGfGmGfAf | | fAmGfUmAmA |
| | | AdCmUfUmAm | | mGdTmUfCmCf |
| | | CmUmAmCmA | | CmAfGmGmCm |
| | | mAmU | | G*mG*mG |
| BBD-2047.512 | BBD-2047SM5 | mC*mG*mCmC | BBD-2047AM12 | mA*fU*mUfGfU |
| | | mUmGfGmGfAf | | fAmGfUmAmAd |
| | | AdCmUfUmAm | | GmUmUfCmCfC |
| | | CmUmAmCmA | | mAfGmGmCmG |
| | | mAmU | | *mG*mG |
| BBD-2047.513 | BBD-2047SM5 | mC*mG*mCmC | BBD-2047AM13 | mA*fU*mUfGfU |
| | | mUmGfGmGfAf | | fAmGfUmAfAm |
| | | AdCmUfUmAm | | GdTmUfCmCfC |
| | | CmUmAmCmA | | mAfGmGmCmG |
| | | mAmU | | *mG*mG |
| BBD-2047.411 | BBD-2047SM4 | mC*mG*mCmC | BBD-2047AM11 | mA*fU*mUfGfU |
| | | mUmGfGmGfAf | | fAmGfUmAmA |
| | | AfCmUfUmAm | | mGdTmUfCmCf |
| | | CmUmAmCmA | | CmAfGmGmCm |
| | | mAmU | | G*mG*mG |
| BBD-2047.52 | BBD-2047SM5 | mC*mG*mCmC | BBD-2047AM2 | mA*fU*mUfGfU |
| | | mUmGfGmGfAf | | fAmGfUmAmA |
| | | AdCmUfUmAm | | mGmUmUfCmC |
| | | CmUmAmCmA | | fCmAfGmGmC |
| | | mAmU | | mG*mG*mG |
| BBD-2047.514 | BBD-2047SM5 | mC*mG*mCmC | BBD-2047AM14 | mA*fU*mUfGfU |
| | | mUmGfGmGfAf | | fAmGfUmAmA |
| | | AdCmUfUmAm | | mGfUmUfCmCf |
| | | CmUmAmCmA | | CmAfGmGmCm |
| | | mAmU | | G*mG*mG |
| BBD-2047.516 | BBD-2047SM5 | mC*mG*mCmC | BBD-2047AM16 | mA*fU*mUfGfU |
| | | mUmGfGmGfAf | | fAmGfUmAfAm |
| | | AdCmUfUmAm | | GfUmUfCmCfC |
| | | CmUmAmCmA | | mAfGmGmCmG |
| | | mAmU | | *mG*mG |
| BBD-2047.517 | BBD-2047SM5 | mC*mG*mCmC | BBD-2047AM17 | mA*fU*mUfGfU |
| | | mUmGfGmGfAf | | fAmGfUmAfAf |
| | | AdCmUfUmAm | | GmUmUfCmCfC |
| | | CmUmAmCmA | | mAfGmGmCmG |
| | | mAmU | | *mG*mG |
| BBD-2047.518 | BBD-2047SM5 | mC*mG*mCmC | BBD-2047AM18 | mA*fU*mUfGfU |
| | | mUmGfGmGfAf | | fAmGfUmAfAm |
| | | AdCmUfUmAm | | GmUmUfCmCfC |
| | | CmUmAmCmA | | mAfGmGmCmG |
| | | mAmU | | *mG*mG |
| BBD-2047.519 | BBD-2047SM5 | mC*mG*mCmC | BBD-2047AM19 | mA*fU*mUfGfU |
| | | mUmGfGmGfAf | | fAmGfUfAmAm |
| | | AdCmUfUmAm | | GmUmUfCmCfC |
| | | CmUmAmCmA | | mAfGmGmCmG |
| | | mAmU | | *mG*mG |
| BBD-2047.520 | BBD-2047SM5 | mC*mG*mCmC | BBD-2047AM20 | mA*fU*mUfGfU |
| | | mUmGfGmGfAf | | fAmGfUmAmA |
| | | AdCmUfUmAm | | mGmUmUfCmC |
| | | CmUmAmCmA | | fCmAmGmGmC |
| | | mAmU | | mG*mG*mG |
| BBD-2047.521 | BBD-2047SM5 | mC*mG*mCmC | BBD-2047AM21 | mA*fU*mUfGfU |
| | | mUmGfGmGfAf | | fAmGfUmAmA |
| | | AdCmUfUmAm | | mGmUmUfCmC |
| | | CmUmAmCmA | | fCmAfGfGmCm |
| | | mAmU | | G*mG*mG |
| BBD-2047.611 | BBD-2047SM6 | mC*mG*mCmC | BBD-2047AM11 | mA*fU*mUfGfU |
| | | mUmGfGmGfA | | fAmGfUmAmA |
| | | mAdCmUfUmA | | mGdTmUfCmCf |
| | | mCmUmAmCm | | CmAfGmGmCm |
| | | AmAmU | | G*mG*mG |
| BBD-2047.612 | BBD-2047SM6 | mC*mG*mCmC | BBD-2047AM12 | mA*fU*mUfGfU |
| | | mUmGfGmGfA | | fAmGfUmAmAd |
| | | mAdCmUfUmA | | GmUmUfCmCfC |
| | | mCmUmAmCm | | mAfGmGmCmG |
| | | AmAmU | | *mG*mG |
| BBD-2047.613 | BBD-2047SM6 | mC*mG*mCmC | BBD-2047AM13 | mA*fU*mUfGfU |
| | | mUmGfGmGfA | | fAmGfUmAfAm |
| | | mAdCmUfUmA | | GdTmUfCmCfC |
| | | mCmUmAmCm | | mAfGmGmCmG |
| | | AmAmU | | *mG*mG |
| BBD-2047.614 | BBD-2047SM6 | mC*mG*mCmC | BBD-2047AM14 | mA*fU*mUfGfU |
| | | mUmGfGmGfA | | fAmGfUmAmA |
| | | mAdCmUfUmA | | mGfUmUfCmCf |
| | | mCmUmAmCm | | CmAfGmGmCm |
| | | AmAmU | | G*mG*mG |
| BBD-2047.615 | BBD-2047SM6 | mC*mG*mCmC | BBD-2047AM15 | mA*fU*mUfGfU |
| | | mUmGfGmGfA | | fAmGfUmAmAf |
| | | mAdCmUfUmA | | GmUmUfCmCfC |
| | | mCmUmAmCm | | mAfGmGmCmG |
| | | AmAmU | | *mG*mG |
| BBD-2047.616 | BBD-2047SM6 | mC*mG*mCmC | BBD-2047AM16 | mA*fU*mUfGfU |
| | | mUmGfGmGfA | | fAmGfUmAfAm |
| | | mAdCmUfUmA | | GfUmUfCmCfC |
| | | mCmUmAmCm | | mAfGmGmCmG |
| | | AmAmU | | *mG*mG |
| BBD-2047.617 | BBD-2047SM6 | mC*mG*mCmC | BBD-2047AM17 | mA*fU*mUfGfU |
| | | mUmGfGmGfA | | fAmGfUmAfAf |
| | | mAdCmUfUmA | | GmUmUfCmCfC |
| | | mCmUmAmCm | | mAfGmGmCmG |
| | | AmAmU | | *mG*mG |
| BBD-2047.618 | BBD-2047SM6 | mC*mG*mCmC | BBD-2047AM18 | mA*fU*mUfGfU |
| | | mUmGfGmGfA | | fAmGfUmAfAm |
| | | mAdCmUfUmA | | GmUmUfCmCfC |
| | | mCmUmAmCm | | mAfGmGmCmG |
| | | AmAmU | | *mG*mG |
| BBD-2047.713 | BBD-2047SM7 | mC*mG*mCmC | BBD-2047AM13 | mA*fU*mUfGfU |
| | | mUmGfGmGfAf | | fAmGfUmAfAm |
| | | AdCmUmUmAm | | GdTmUfCmCfC |
| | | CmUmAmCmA | | mAfGmGmCmG |
| | | mAmU | | *mG*mG |
| BBD-2047.716 | BBD-2047SM7 | mC*mG*mCmC | BBD-2047AM16 | mA*fU*mUfGfU |
| | | mUmGfGmGfAf | | fAmGfUmAfAm |
| | | AdCmUmUmAm | | GfUmUfCmCfC |
| | | CmUmAmCmA | | mAfGmGmCmG |
| | | mAmU | | *mG*mG |
| BBD-2047.72 | BBD-2047SM7 | mC*mG*mCmC | BBD-2047AM2 | mA*fU*mUfGfU |
| | | mUmGfGmGfAf | | fAmGfUmAmA |
| | | AdCmUmUmAm | | mGmUmUfCmC |
| | | CmUmAmCmA | | fCmAfGmGmC |
| | | mAmU | | mG*mG*mG |
| BBD-2047.723 | BBD-2047SM7 | mC*mG*mCmC | BBD-2047AM23 | mA*fU*mUmG |
| | | mUmGfGmGfAf | | mUmAmGmUm |
| | | AdCmUmUmAm | | AfAmGmUmUf |
| | | CmUmAmCmA | | CmCfCmAmGm |
| | | mAmU | | GmCmG*mG*m |
| | | | | G |
| BBD-2047.823 | BBD-2047SM8 | mC*mG*mCmC | BBD-2047AM23 | mA*fU*mUmG |
| | | mUmGfGmGfAf | | mUmAmGmUm |
| | | AfCmUmUmAm | | AfAmGmUmUf |
| | | CmUmAmCmA | | CmCfCmAmGm |
| | | mAmU | | GmCmG*mG*m |
| | | | | G |
| BBD-2047.824 | BBD-2047SM8 | mC*mG*mCmC | BBD-2047AM24 | mA*fU*mUmG |
| | | mUmGfGmGfAf | | mUmAmGmUm |
| | | AfCmUmUmAm | | AfAmGfUmUfC |
| | | CmUmAmCmA | | mCfCmAmGmG |
| | | mAmU | | mCmG*mG*mG |
| BBD-2047.81 | BBD-2047SM8 | mC*mG*mCmC | BBD-2047AM1 | mA*fU*mUmG |
| | | mUmGfGmGfAf | | mUmAmGmUm |
| | | AfCmUmUmAm | | AfAmGdTmUfC |
| | | CmUmAmCmA | | mCfCmAmGmG |
| | | mAmU | | mCmG*mG*mG |
| BBD-2047.522 | BBD-2047SM5 | mC*mG*mCmC | BBD-2047AM22 | mA*fU*mUfGfU |
| | | mUmGfGmGfAf | | fAmGfUmAfAm |
| | | AdCmUfUmAm | | GmUmUfCmCfC |
| | | CmUmAmCmA | | mAmGmGmCm |
| | | mAmU | | G*mG*mG |
| BBD-2047.718 | BBD-2047SM7 | mC*mG*mCmC | BBD-2047AM18 | mA*fU*mUfGfU |
| | | mUmGfGmGfAf | | fAmGfUmAfAm |
| | | AdCmUmUmAm | | GmUmUfCmCfC |
| | | CmUmAmCmA | | mAfGmGmCmG |
| | | mAmU | | *mG*mG |
| BBD-2047.720 | BBD-2047SM7 | mC*mG*mCmC | BBD-2047AM20 | mA*fU*mUfGfU |
| | | mUmGfGmGfAf | | fAmGfUmAmA |
| | | AdCmUmUmAm | | mGmUmUfCmC |
| | | CmUmAmCmA | | fCmAmGmGmC |
| | | mAmU | | mG*mG*mG |
| BBD-2047.722 | BBD-2047SM7 | mC*mG*mCmC | BBD-2047AM22 | mA*fU*mUfGfU |
| | | mUmGfGmGfAf | | fAmGfUmAfAm |
| | | AdCmUmUmAm | | GmUmUfCmCfC |
| | | CmUmAmCmA | | mAmGmGmCm |
| | | mAmU | | G*mG*mG |
| BBD-2047.82 | BBD-2047SM8 | mC*mG*mCmC | BBD-2047AM2 | mA*fU*mUfGfU |
| | | mUmGfGmGfAf | | fAmGfUmAmA |
| | | AfCmUmUmAm | | mGmUmUfCmC |
| | | CmUmAmCmA | | fCmAfGmGmC |
| | | mAmU | | mG*mG*mG |
| BBD-2047.822 | BBD-2047SM8 | mC*mG*mCmC | BBD-2047AM22 | mA*fU*mUfGfU |
| | | mUmGfGmGfAf | | fAmGfUmAfAm |
| | | AfCmUmUmAm | | GmUmUfCmCfC |
| | | CmUmAmCmA | | mAmGmGmCm |
| | | mAmU | | G*mG*mG |
| BBD-2047.923 | BBD-2047SM9 | mC*mG*mCmC | BBD-2047AM23 | mA*fU*mUmG |
| | | mUmGfGmGfAd | | mUmAmGmUm |
| | | AfCmUmUmAm | | AfAmGmUmUf |
| | | CmUmAmCmA | | CmCfCmAmGm |
| | | mAmU | | GmCmG*mG*m |
| | | | | G |
| BBD-2047.102 | BBD- | mC*mG*mCmC | BBD-2047AM23 | mA*fU*mUmG |
| 3 | 2047SM10 | mUmGfGmGdAf | | mUmAmGmUm |
| | | AfCmUmUmAm | | AfAmGmUmUf |
| | | CmUmAmCmA | | CmCfCmAmGm |
| | | mAmU | | GmCmG*mG*m |
| | | | | G |
| BBD-2047.825 | BBD-2047SM8 | mC*mG*mCmC | BBD-2047AM25 | mA*fU*mUmG |
| | | mUmGfGmGfAf | | mUmAmGmUm |
| | | AfCmUmUmAm | | AfAdGmUmUfC |
| | | CmUmAmCmA | | mCfCmAmGmG |
| | | mAmU | | mCmG*mG*mG |
| BBD-2047.826 | BBD-2047SM8 | mC*mG*mCmC | BBD-2047AM26 | mA*fU*mUmG |
| | | mUmGfGmGfAf | | mUmAmGmUm |
| | | AfCmUmUmAm | | AfAmGmUdTfC |
| | | CmUmAmCmA | | mCfCmAmGmG |
| | | mAmU | | mCmG*mG*mG |

Table 23 describes the experimental results of multiple modified BBD-2047 double-stranded siRNA sequences delivered via GalNAc at a concentration of 10 nM in primary hepatocytes derived from hTMPRSS6-expressing mice.

| | |
|---|---|
| | hTMPRSS6 mRNA level in primary |
| | mouse hepatocytes (%) (relative to |
| siRNA | negative control cells) |

| Duplex | 10 nM avg | SD |
|---|---|---|
| TMPRSS6-hcm-9- | 13.87 | 1.86 |
| Galnac | | |
| BBD-2047.14-Galnac | 6.03 | 1.13 |
| BBD-2047.13-Galnac | 17.90 | 0.79 |
| BBD-2047.12-Galnac | 4.37 | 0.89 |
| BBD-2047.42-Galnac | 4.09 | 0.76 |
| BBD-2047.43-Galnac | 31.04 | 6.71 |
| BBD-2047.53-Galnac | 34.47 | 3.22 |
| BBD-2047.55-Galnac | 18.63 | 1.62 |
| BBD-2047.56-Galnac | 18.57 | 0.32 |
| BBD-2047.57-Galnac | 43.98 | 14.65 |
| BBD-2047.58-Galnac | 48.34 | 12.99 |
| BBD-2047.59-Galnac | 40.37 | 16.07 |
| BBD-2047.510-Galnac | 33.04 | 1.95 |
| BBD-2047.511-Galnac | 4.22 | 0.49 |
| BBD-2047.512-Galnac | 6.08 | 2.02 |
| BBD-2047.513-Galnac | 4.25 | 1.89 |

Table 24 describes the experimental results of multiple modified BBD-2047 double-stranded siRNA sequences delivered via GalNAc at a concentration of 5 nM in primary hepatocytes derived from hTMPRSS6-expressing mice.

| | hTMPRSS6 mRNA level in primary mouse | |
|---|---|---|
| | hepatocytes (%) (relative to negative | |
| siRNA | control cells) | |
| Duplex | 5 nM avg | SD |
| TMPRSS6-hcm-9- | 20.97 | 1.26 |
| Galnac | | |
| BBD-2047.42-Galnac | 5.78 | 1.91 |
| BBD-2047.411-Galnac | 9.79 | 6.34 |
| BBD-2047.52-Galnac | 2.88 | 1.51 |
| BBD-2047.511-Galnac | 17.68 | 6.29 |
| BBD-2047.513-Galnac | 6.11 | 3.97 |
| BBD-2047.514-Galnac | 15.70 | 6.19 |
| BBD-2047.516-Galnac | 7.50 | 1.83 |
| BBD-2047.517-Galnac | 19.90 | 0.76 |
| BBD-2047.518-Galnac | 0.83 | 0.77 |
| BBD-2047.519-Galnac | 2.08 | 0.60 |
| BBD-2047.520-Galnac | 1.25 | 1.62 |
| BBD-2047.521-Galnac | 3.41 | 1.75 |
| BBD-2047.611-Galnac | 12.77 | 3.84 |
| BBD-2047.612-Galnac | 29.73 | 1.21 |
| BBD-2047.613-Galnac | 32.68 | 6.36 |
| BBD-2047.614-Galnac | 23.82 | 11.44 |
| BBD-2047.615-Galnac | 33.94 | 9.72 |
| BBD-2047.616-Galnac | 22.24 | 12.59 |
| BBD-2047.617-Galnac | 29.30 | 4.28 |
| BBD-2047.618-Galnac | 30.85 | 12.61 |
| BBD-2047.713-Galnac | 2.93 | 1.50 |
| BBD-2047.716-Galnac | 2.72 | 2.84 |
| BBD-2047.72-Galnac | 1.18 | 0.61 |
| BBD-2047.723-Galnac | 0.67 | 0.37 |
| BBD-2047.823-Galnac | 1.50 | 0.62 |
| BBD-2047.824-Galnac | 0.73 | 0.15 |
| BBD-2047.81-Galnac | 3.91 | 1.12 |

Table 25 describes the experimental results of multiple modified BBD-2047 double-stranded siRNA sequences delivered via GalNAc at a concentration of 1 nM in primary hepatocytes derived from hTMPRSS6-expressing mice.

| | |
|---|---|
| | hTMPRSS6 mRNA level in primary |
| | mouse hepatocytes (%) (relative to |
| siRNA | negative control cells) |

| Duplex | 1 nM avg | SD |
|---|---|---|
| TMPRSS6-hcm-9- | 27.69 | 8.97 |
| Galnac | | |
| BBD-2047.52-Galnac | 9.02 | 7.06 |
| BBD-2047.522-Galnac | 13.78 | 8.71 |
| BBD-2047.72-Galnac | 10.44 | 3.25 |
| BBD-2047.718-Galnac | 16.27 | 3.47 |
| BBD-2047.723-Galnac | 5.28 | 1.79 |
| BBD-2047.720-Galnac | 39.13 | 11.85 |
| BBD-2047.722-Galnac | 7.19 | 3.85 |
| BBD-2047.72-Galnac | 13.88 | 1.70 |
| BBD-2047.82-Galnac | 12.11 | 6.81 |
| BBD-2047.822-Galnac | 11.31 | 2.81 |
| BBD-2047.823-Galnac | 5.27 | 2.68 |
| BBD-2047.824-Galnac | 10.75 | 1.63 |

Table 26 describes the experimental results of multiple modified BBD-2047 double-stranded siRNA sequences delivered via GalNAc at a concentration of 0.5 nM in primary hepatocytes derived from hTMPRSS6-expressing mice.

| | |
|---|---|
| | hTMPRSS6 mRNA level in |
| | primary mouse hepatocytes |
| | (%) (relative to negative |
| siRNA | control cells) |

| Duplex | 0.5 nM avg | SD |
|---|---|---|
| TMPRSS6-hcm-9- | 57.20 | 16.97 |
| Galnac | | |
| BBD-2047.52-Galnac | 87.16 | 35.19 |
| BBD-2047.518-Galnac | 23.75 | 12.87 |
| BBD-2047.520-Galnac | 26.51 | 12.58 |
| BBD-2047.72-Galnac | 18.07 | 4.88 |
| BBD-2047.716-Galnac | 78.41 | 6.72 |
| BBD-2047.722-Galnac | 28.27 | 28.46 |
| BBD-2047.723-Galnac | 19.50 | 2.07 |
| BBD-2047.823-Galnac | 23.49 | 25.06 |
| BBD-2047.824-Galnac | 10.01 | 3.03 |

Table 27 describes the experimental results of modified BBD-2047 double-stranded siRNA sequences and TMPRSS6-HCM-9 screened at a concentration of 10 nM in Hep3B cells.

hTMPRSS6 mRNA level in Hep3B cells
(%) (relative to negative control cells)

| siRNA Duplex | 10 nM avg | SD |
|---|---|---|
| TMPRSS6-hcm-9 | 21.53 | 2.60 |
| BBD-2047.723 | 17.90 | 2.38 |
| BBD-2047.923 | 18.76 | 1.39 |
| BBD-2047.1023 | 18.16 | 1.78 |

### Example 4: Evaluation of IC50 for Inhibition of hTMPRSS6 Expression by Different Sequences in Hep3B Cells

Cell culture and 96-Well plate transfection: In vitro experiments were conducted using Hep3B cells, which were cultured in MEM supplemented with 10% FBS, 1X penicillin-streptomycin (Gibco, Cat.15070-063), and 1X non-essential amino acids (Cell Cook, Cat.CM1008L). When the cell confluence reached 80%, the cells were digested with trypsin, and the cell density was measured using a Scepter automated cell counter (Millipore, #PHCC00000). Meanwhile, siRNA, Opti-MEM, and INTERFERin (Polyplus Transfection) were mixed in a 96-well plate and incubated at room temperature for 10 minutes. Complete culture medium with Hep3B cells was then added to each well, and the 96-well plate was incubated at 37° C. in a 5% CO₂ incubator for 24 hours.

siRNA was tested at a maximum concentration of 100 nM, with eight concentration points prepared by three-fold serial dilution.

RNA extraction and reverse transcription from a 96-well plate: mRNA was extracted from cells in the 96-well plate using the Dynabeads mRNA DIRECT Kit (Ambion). The culture medium was aspirated, and the wells were rinsed once with DPBS. Then, 50-300 μL of cell lysis buffer was added to each well, followed by 20-100 μL of beads. The plate was placed on a shaker for thorough mixing. Next, the 96-well plate was placed on a magnetic stand, and the lysis buffer was aspirated. Each well was then washed with 50-300 μL of Wash Buffer A, mixed thoroughly, and placed on the magnetic stand to remove the buffer. The beads were then resuspended in Wash Buffer B, transferred to a new 96-well plate, and placed on the magnetic stand to remove the buffer. The beads were once again resuspended in Wash Buffer B and transferred to a 96-well PCR plate. Meanwhile, reverse transcription reagents were prepared. The 96-well PCR plate was placed on a magnetic stand, and the wash buffer was aspirated. Then, 20 μL of reverse transcription reagent was added to each well, and the plate was sealed with sealing film. The plate was incubated at 25° C. for 10 minutes on a PCR instrument, then at 37° C. for 2 hours, followed by 85° C. for 5 minutes, and finally cooled to 4° C. to complete the reverse transcription process.

Real-time fluorescent quantitative PCR (qPCR): After reverse transcription, the 96-well plate was placed on a magnetic stand until the beads were completely adsorbed to the bottom. The reverse transcription reagent was then aspirated, and the prepared qPCR reaction mixture was added to the 96-well PCR plate. The plate was sealed with a sealing film and subjected to qPCR analysis using the StepOnePlus Real-Time PCR System (Applied Biosystems).

The ΔΔCt method was used to analyze the data, and the test was normalized using cells transfected with a 1 nM negative control sequence.

Table 28 describes the activity results of different modified double-stranded sequences in Hep3B cells.

| | HEP3B(n = 3) | | | |
|---|---|---|---|---|
| siRNA duplex | BOTTOM | Top | SLOPE | IC₅₀ (nM) |
| TMPRSS6-hcm-9- | 27.6 | 93.9 | −1.1 | 0.442 |
| Galnac | | | | |
| BBD-2083.415-Galnac | 24.0 | =100.0 | −1.1 | 0.07841 |
| BBD-2051.210-Galnac | 18.8 | =100.0 | −1.4 | 0.03669 |

### Example 5: Evaluation of the Effect of Different Modified BBD-2047, BBD-2051, and BBD-2083 Sequences on hTMPRSS6 Expression in the Liver of hTMPRSS6-Expressing Mice

1. Integration of hTMPRSS6 via Adenovirus

Mice were infected with 1×10¹¹ to 10×10¹¹ purified recombinant AAV8 viral particles to generate transgenic mice with stable hTMPRSS6 expression.

### 2. Drug Administration

Fourteen days after viral injection, the mice were evenly divided into groups, with four mice per group. The siRNA was dissolved in saline and administered via subcutaneous injection at doses of 1 mg/kg and 3 mg/kg.

### 3. Liver Sampling and Detection

At different time points after siRNA injection, liver samples were collected. Total RNA was extracted from liver tissue using TRI REAGENT (MRC, Cat.No. TR118), and the extracted RNA was reverse transcribed into cDNA using the PrimeScript RT Reagent Kit (Takara, Cat. RR047A). The prepared qPCR reaction mixture was added to a 96-well PCR plate, sealed, and subjected to qPCR analysis using the StepOnePlus Real-Time PCR System (Applied Biosystems) to quantify hTMPRSS6 expression.

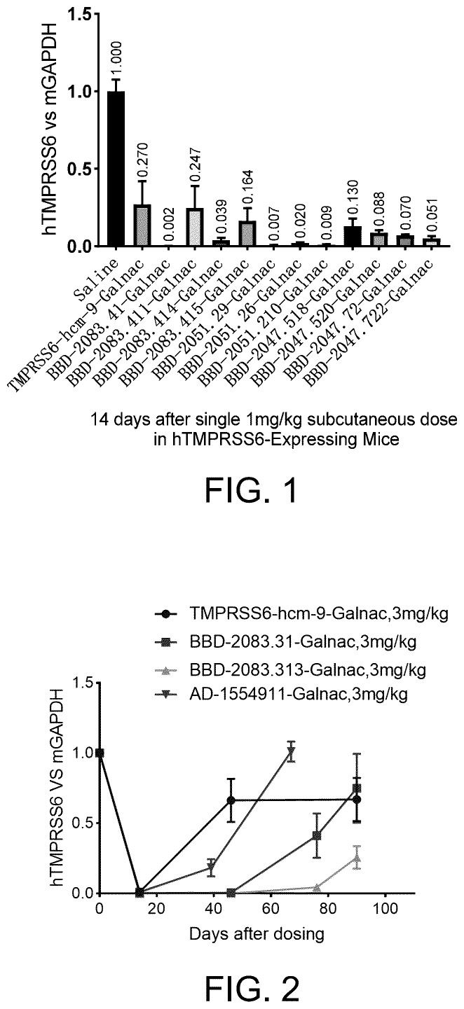

Fourteen days after siRNA injection, hTMPRSS6 expression levels were measured, and the results are shown in FIG. 1. At a dose of 1 mg/kg, different modified sequences exhibited varied in vivo activity, with multiple sequences demonstrating significant gene silencing effects in mice, showing greater activity of TMPRSS6-hcm-9-GalNAc.

At different time points after siRNA injection, hTMPRSS6 expression levels were measured, and the results are shown in FIG. 2. At a dose of 3 mg/kg, BBD-2083.31-GalNAc and BBD-2083.313-GalNAc exhibited a longer duration of pharmacodynamic effects compared to TMPRSS6-hcm-9-GalNAc and AD-1554911-GalNAc. Additionally, we observed that incorporating a VP modification into BBD-2083 further enhanced its in vivo activity (BBD-2083.313-GalNAc vs. BBD-2083.31-GalNAc).

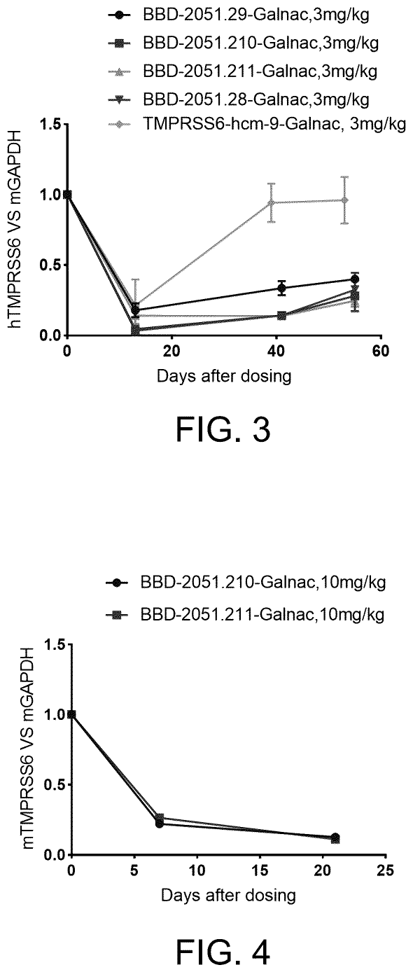

At different time points after siRNA injection, hTMPRSS6 expression levels were measured, and the results are shown in FIG. 3. At a dose of 3 mg/kg, all modified BBD-2051 sequences exhibited high activity, with a longer duration of pharmacodynamic effect compared to TMPRSS6-hcm-9-GalNAc.

### Example 6: Evaluation of the Effects of Different Modified BBD-2051 Sequences on mTMPRSS6 and mHepcidin1 Expression in the Liver and Serum Iron Levels in Wild-Type Mice

Wild-type mice were subcutaneously administered 10 mg/kg of siRNA, and liver samples were collected on days 7 and 21. Total RNA was extracted from liver tissue using TRI REAGENT (MRC, Cat.No. TR118), and the extracted RNA was reverse transcribed into cDNA using the PrimeScript RT reagent Kit (Takara, Cat. RR047A). The prepared qPCR reaction mixture was added to a 96-well PCR plate, sealed, and subjected to qPCR analysis using the StepOnePlus Real-Time PCR System (Applied Biosystems) to quantify mTMPRSS6 expression levels. The results are shown in FIG. 4. The findings indicate that BBD-2051.210 and BBD-2051.211 effectively reduced endogenous TMPRSS6 expression in wild-type mice.

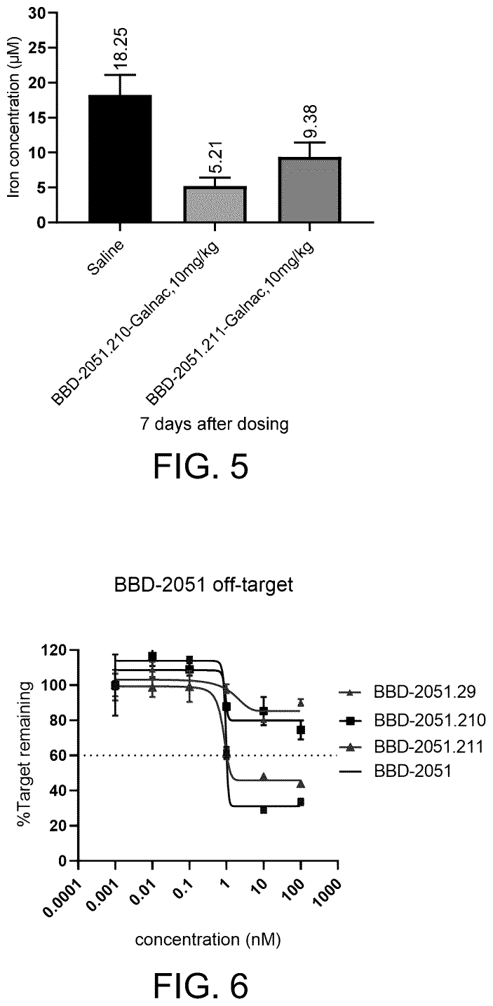

Wild-type mice were subcutaneously administered 10 mg/kg of siRNA. On day 7, a small amount of blood was collected from the tail. The blood was allowed to clot at room temperature for 30 minutes, then centrifuged at 1000×g for 10 minutes, and the supernatant serum was collected. The serum was diluted and processed using the Iron Assay Kit-Colorimetric according to the manufacturer's instructions. Reducer solution was added, and the samples were incubated at 37° C. for 15 minutes, followed by the addition of 100 μL of Probe Solution, and further incubation at 37° C. for 1 hour. The absorbance at 593 nm was measured, and serum iron concentration was calculated based on a standard curve. The results are shown in FIG. 5. The findings indicate that BBD-2051.210 and BBD-2051.211 effectively reduced serum iron levels in wild-type mice.

### Example 7: Evaluation of Off-Target Activity of Different Modified BBD-2051 and BBD-2083 Sequences

siRNA-specific off-target sequences were designed, synthesized, and cloned into the psiCheck2 vector. The psiCheck2-Off-target plasmid was co-transfected with siRNA in 24-well plates. Prior to transfection, cells were seeded in 0.5 mL of growth medium per well for 6-8 hours. Using a transfection reagent, 50 ng of psiCheck2-Off-target plasmid and siRNA at concentrations ranging from 0.0001 nM to 100 nM were transfected and incubated at 37° C. for 24 hours.

Fluorescence signal values of Renilla luciferase and firefly luciferase were measured using the Dual-Luciferase Reporter Assay System (Promega, Cat #E1980). The normalized value was calculated as follows:

Normalized value=Renilla luciferase reading/Firefly luciferase reading

Relative expression fold change=Normalized value of the experimental group/Normalized value of the control group

Experimental results, as shown in FIG. 6, indicate that introducing ISOGNA at positions 5, 6, and 7 of the antisense strand significantly reduced off-target activity. In vitro experiments demonstrated that the modified BBD-2051 sequences (particularly BBD-2051.29 and BBD-2051.210) exhibited minimal off-target activity and a favorable safety profile.

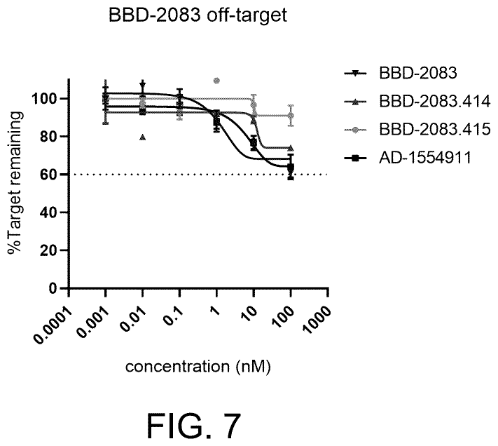

As shown in FIG. 7, in vitro experimental results demonstrated that the modified BBD-2083 sequences exhibited minimal off-target activity and a favorable safety profile.

### Example 8: Evaluation of the In Vitro Metabolic Stability of BBD-2051.210 in Serum and Liver S9 Fractions

BBD-2051.210-GalNAc was incubated in vitro at 37° C. for 24 hours with serum or liver S9 fractions derived from mice, rats, dogs, monkeys, and humans to evaluate its metabolic stability. Experimental data, as shown in Table 29, indicate that in serum from different species, the stability ranking of the antisense strand was mouse>monkey>rat>human>dog, while the stability ranking of the sense strand was monkey>rat>mouse>human>dog. As shown in Table 30, in liver S9 fractions from different species, the stability ranking of the antisense strand was dog>human>monkey>mouse>rat, while the stability ranking of the sense strand was dog>mouse>human>monkey>rat.

TABLE 29
Serum Stability Results of BBD-2051.210-GalNAc

| 24 h residual | | | | | |
|---|---|---|---|---|---|
| rate(%) serum | mouse | rat | dog | monkey | human |
| Antisense | 102.99 | 91.03 | 71.30 | 96.87 | 87.35 |
| sense | 94.63 | 95.28 | 71.76 | 98.94 | 87.92 |

TABLE 30
Liver S9 Stability Results of BBD-2051.210-GalNAc

| 24 h residual | | | | | |
|---|---|---|---|---|---|
| rate(%) liver S9 | mouse | rat | dog | monkey | human |
| Antisense | 83.47 | 74.37 | 104.55 | 95.17 | 95.31 |
| sense | 99.47 | 85.95 | 105.21 | 95.40 | 96.02 |

### Example 9: Evaluation of the In Vitro Metabolic Stability of BBD-2051.210 in Liver Homogenate

BBD-2051.210-GalNAc was incubated in vitro at 37° C. for 72 hours with rat liver homogenate to evaluate its metabolic stability. After 72 hours of incubation, the remaining percentage of the antisense strand was 94.98%, while the remaining percentage of the sense strand was 54.24%. These data indicate that BBD-2051.210-GalNAc exhibits stability in liver homogenate.

The embodiments described above represent only certain implementations of the present invention. While provided in detail, they should not be interpreted as limiting the scope of the invention. Those skilled in the art may make various modifications, adaptations, or improvements without departing from the spirit and scope of the invention. Accordingly, the protection scope of the present invention shall be defined solely by the appended claims.

What is claimed is:

1. An siRNA or a pharmaceutically acceptable salt thereof for inhibiting the expression of TMPRSS6 in a cell, comprising a sense strand and an antisense strand, wherein the nucleotide sequence is selected from any one of (a) to (e) as set forth herein:
(a) BBD-2051, wherein the sense strand sequence is as set forth in SEQ ID NO:103, and the antisense strand sequence is as set forth in SEQ ID NO:104;
(b) BBD-2083, wherein the sense strand sequence is as set forth in SEQ ID NO:167, and the antisense strand sequence is as set forth in SEQ ID NO:168;
(c) BBD-2047, wherein the sense strand sequence is as set forth in SEQ ID NO:95, and the antisense strand sequence is as set forth in SEQ ID NO:96;
(d) BBD-2086, wherein the sense strand sequence is as set forth in SEQ ID NO:173, and the antisense strand sequence is as set forth in SEQ ID NO:174;
(e) BBD-2087, wherein the sense strand sequence is as set forth in SEQ ID NO:175, and the antisense strand sequence is as set forth in SEQ ID NO:176.

2. The siRNA or the pharmaceutically acceptable salt thereof of claim 1, wherein the sense strand comprises no more than 3, 2, 1, or 0 unmodified nucleotides, wherein the modified nucleotides in the sense strand are independently selected from 2′-O-methyl-modified nucleotides, 2′-deoxynucleotides, and 2′-fluoro-modified nucleotides; wherein the sense strand comprises 1, 2, or 3 phosphorothioate linkages at the 5′-terminus; and wherein the 2′-deoxynucleotide is selected from at least one of the following structures: 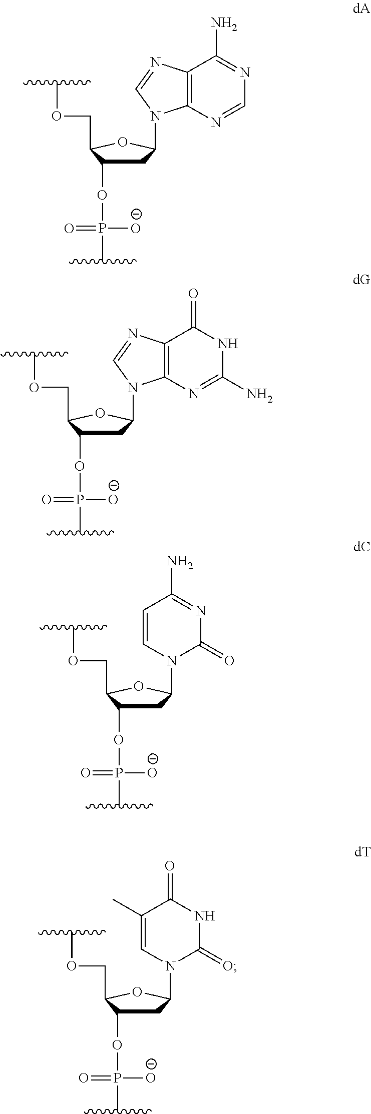
wherein the antisense strand comprises no more than 3, 2, 1, or 0 unmodified nucleotides, wherein the modified nucleotides in the antisense strand are independently selected from 2′-O-methyl-modified nucleotides, 2′-deoxynucleotides, 2′-fluoro-modified nucleotides, 5′-(E)-vinylphosphonate nucleotides, and isomer of glycerol nucleic acid (isoGNA); wherein the antisense strand comprises 1 to 3 phosphorothioate linkages at both the 5′-terminus and the 3′-terminus; and wherein isomer of glycerol nucleic acid (isoGNA) is selected from one of the following structures: 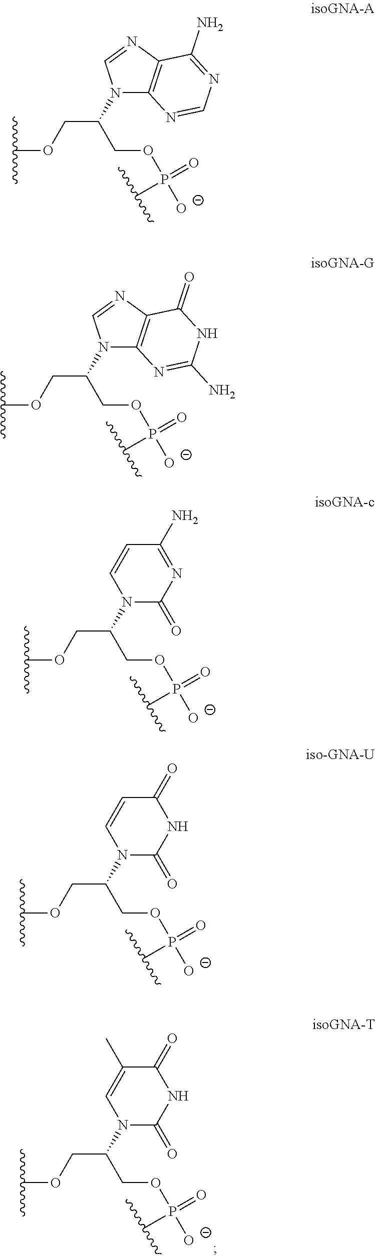
wherein, the structure of the uridine-5′-(E)-vinylphosphonate nucleotides is as follows:

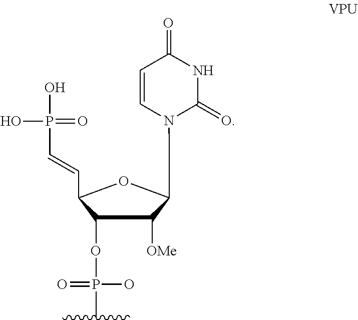

3. The siRNA or the pharmaceutically acceptable salt thereof of claim 2, wherein the isoGNA is located in at least one position between position 4 and 8 counted from the 5′-terminus of the antisense strand.

4. The siRNA or the pharmaceutically acceptable salt thereof of claim 1, wherein at least one strand of the siRNA comprises a 3′-overhang of at least 1 to 3 nucleotides; and wherein antisense strand of the complementary siRNA at positions 11 to 13 of the 5′-terminus and/or the sense strand of the complementary siRNA at positions 9 to 11 of the 5′-terminus together comprise a total of 1 to 6 2′-deoxynucleotides.

5. The siRNA or the pharmaceutically acceptable salt thereof of claim 1, wherein the modified siRNA is selected from the group consisting of:
BBD-2051.210:
Sense strand: mU*mG*mGmGmAmAfCmUfUfAfCmUmAmCmAmAmCmUmCmCmA,
Antisense strand: VPU*fG*mGmAmGisoGNA-TmUmGmUfAmGdTmAfAmGfUmUmCmCmCmA*mG*mG,
BBD-2051.211:
Sense strand: mU*mG*mGmGmAmAfCmUfUfAfCmUmAmCmAmAmCmUmCmCmA,
Antisense strand: VPU*fG*mGmAmGmUisoGNA-TmGmUfAmGdTmAfAmGfUmUmCmCmCmA*mG*mG,
BBD-2051.28:
Sense strand: mU*mG*mGmGmAmAfCmUfUfAfCmUmAmCmAmAmCmUmCmCmA,
Antisense strand: VPU*fG*mGmAmGmUmUmGmUfAmGdTmAfAmGfUmUmCmCmCmA*mG*mG,
BBD-2083.313:
Sense strand: mU*mG*mCmUmAmCfUmCfUfGfGmUmAmUmUmUmCmCmUmAmA,
Antisense strand: VPU*fU*mAmGmGmAmAmAmUfAmCdCmAfGmAfGmUmAmGmCmA*mC*mC,
BBD-2083.413:
Sense strand: mU*mG*mCmUmAmCfUmCfUfGdGmUmAmUmUmUmCmCmUmAmA,
Antisense strand: VPU*fU*mAmGmGmAmAmAmUfAmCdCmAfGmAfGmUmAmGmCmA*mC*mC,
BBD-2051.25:
Sense strand: mU*mG*mGmGmAmAfCmUfUfAfCmUmAmCmAmAmCmUmCmCmA,
Antisense strand: mU*fG*mGmAisoGNA-GmUmUmGmUfAmGdTmAfAmGfUmUmCmCmCmA*mG*mG,
BBD-2051.26:
Sense strand: mU*mG*mGmGmAmAfCmUfUfAfCmUmAmCmAmAmCmUmCmCmA,
Antisense strand: mU*fG*mGmAmGisoGNA-TmUmGmUfAmGdTmAfAmGfUmUmCmCmCmA*mG*mG,
BBD-2051.27:
Sense strand: mU*mG*mGmGmAmAfCmUfUfAfCmUmAmCmAmAmCmUmCmCmA,
Antisense strand: mU*fG*mGmAmGmUisoGNA-TmGmUfAmGdTmAfAmGfUmUmCmCmCmA*mG*mG,
BBD-2051.29:
Sense strand: mU*mG*mGmGmAmAfCmUfUfAfCmUmAmCmAmAmCmUmCmCmA,
Antisense strand: VPU*fG*mGmAisoGNA-GmUmUmGmUfAmGdTmAfAmGfUmUmCmCmCmA*mG*mG,
BBD-2051.213:
Sense strand: mU*mG*mGmGmAmAfCmUfUfAfCmUmAmCmAmAmCmUmCmCmA,
Antisense strand: mU*dG*mGmAisoGNA-GmUmUmGmUmAmGdTmAfAmGfUmUmCmCmCmA*mG*mG,
BBD-2051.214:
Sense strand: mU*mG*mGmGmAmAfCmUfUfAfCmUmAmCmAmAmCmUmCmCmA,
Antisense strand: mU*dG*mGmAmGisoGNA-TmUmGmUmAmGdTmAfAmGfUmUmCmCmCmA*mG*mG,
BBD-2051.215:
Sense strand: mU*mG*mGmGmAmAfCmUfUfAfCmUmAmCmAmAmCmUmCmCmA,
Antisense strand: mU*dG*mGmAmGmUisoGNA-TmGmUmAmGdTmAfAmGfUmUmCmCmCmA*mG*mG,
BBD-2051.217:
Sense strand: mU*mG*mGmGmAmAfCmUfUfAfCmUmAmCmAmAmCmUmCmCmA,
Antisense strand: VPU*dG*mGmAisoGNA-GmUmUmGmUmAmGdTmAfAmGfUmUmCmCmCmA*mG*mG,
BBD-2051.218:
Sense strand: mU*mG*mGmGmAmAfCmUfUfAfCmUmAmCmAmAmCmUmCmCmA,
Antisense strand: VPU*dG*mGmAmGisoGNA-TmUmGmUmAmGdTmAfAmGfUmUmCmCmCmA*mG*mG,
BBD-2051.219:
Sense strand: mU*mG*mGmGmAmAfCmUfUfAfCmUmAmCmAmAmCmUmCmCmA,
Antisense strand: VPU*dG*mGmAmGmUisoGNA-TmGmUmAmGdTmAfAmGfUmUmCmCmCmA*mG*mG,
BBD-2083.41:
Sense strand: mU*mG*mCmUmAmCfUmCfUfGdGmUmAmUmUmUmCmCmUmAmA,
Antisense strand: mU*fU*mAmGmGmAmAmAmUfAmCdCmAfGmAfGmUmAmGmCmA*mC*mC,
BBD-2083.410:
Sense strand: mU*mG*mCmUmAmCfUmCfUfGdGmUmAmUmUmUmCmCmUmAmA,
Antisense strand: mU*fU*mAmGisoGNA-GmAmAmAmUfAmCdCmAfGmAfGmUmAmGmCmA*mC*mC,
BBD-2083.411:
Sense strand: mU*mG*mCmUmAmCfUmCfUfGdGmUmAmUmUmUmCmCmUmAmA,
Antisense strand: mU*fU*mAmGmGisoGNA-AmAmAmUfAmCdCmAfGmAfGmUmAmGmCmA*mC*mC,
BBD-2083.412:
Sense strand: mU*mG*mCmUmAmCfUmCfUfGdGmUmAmUmUmUmCmCmUmAmA,
Antisense strand: mU*fU*mAmGmGmAisoGNA-AmAmUfAmCdCmAfGmAfGmUmAmGmCmA*mC*mC,
BBD-2083.414:
Sense strand: mU*mG*mCmUmAmCfUmCfUfGdGmUmAmUmUmUmCmCmUmAmA,
Antisense strand: VPU*fU*mAmGisoGNA-GmAmAmAmUfAmCdCmAfGmAfGmUmAmGmCmA*mC*mC,
BBD-2083.415:
Sense strand: mU*mG*mCmUmAmCfUmCfUfGdGmUmAmUmUmUmCmCmUmAmA,
Antisense strand: VPU*fU*mAmGmGisoGNA-AmAmAmUfAmCdCmAfGmAfGmUmAmGmCmA*mC*mC,
BBD-2083.416:
Sense strand: mU*mG*mCmUmAmCfUmCfUfGdGmUmAmUmUmUmCmCmUmAmA,
Antisense strand: VPU*fU*mAmGmGmAisoGNA-AmAmUfAmCdCmAfGmAfGmUmAmGmCmA*mC*mC,
BBD-2083.418:
Sense strand: mU*mG*mCmUmAmCfUmCfUfGdGmUmAmUmUmUmCmCmUmAmA,
Antisense strand: mU*dT*mAmGisoGNA-GmAmAmAmUmAmCdCmAfGmAfGmUmAmGmCmA*mC*mC,
BBD-2083.419:
Sense strand: mU*mG*mCmUmAmCfUmCfUfGdGmUmAmUmUmUmCmCmUmAmA,
Antisense strand: mU*dT*mAmGmGisoGNA-AmAmAmUmAmCdCmAfGmAfGmUmAmGmCmA*mC*mC,
BBD-2083.420:
Sense strand: mU*mG*mCmUmAmCfUmCfUfGdGmUmAmUmUmUmCmCmUmAmA,
Antisense strand: mU*dT*mAmGmGmAisoGNA-AmAmUmAmCdCmAfGmAfGmUmAmGmCmA*mC*mC,
BBD-2083.422:
Sense strand: mU*mG*mCmUmAmCfUmCfUfGdGmUmAmUmUmUmCmCmUmAmA,
Antisense strand: VPU*dT*mAmGisoGNA-GmAmAmAmUmAmCdCmAfGmAfGmUmAmGmCmA*mC*mC,
BBD-2083.423:
Sense strand: mU*mG*mCmUmAmCfUmCfUfGdGmUmAmUmUmUmCmCmUmAmA,
Antisense strand: VPU*dT*mAmGmGisoGNA-AmAmAmUmAmCdCmAfGmAfGmUmAmGmCmA*mC*mC,
BBD-2083.424:
Sense strand: mU*mG*mCmUmAmCfUmCfUfGdGmUmAmUmUmUmCmCmUmAmA,
Antisense strand: VPU*dT*mAmGmGmAisoGNA-AmAmUmAmCdCmAfGmAfGmUmAmGmCmA*mC*mC.

6. The siRNA or the pharmaceutically acceptable salt thereof of claim 1, wherein the pharmaceutically acceptable salt is selected from a sodium salt or a potassium salt.

7. A biological agent or pharmaceutical composition for inhibiting the expression of TMPRSS6, wherein, the biological agent or pharmaceutical composition comprises the siRNA or the pharmaceutically acceptable salt thereof of claim 1, and a targeting delivery ligand or delivery vehicle conjugated to or encapsulating the siRNA.

8. The biological agent or pharmaceutical composition of claim 7, wherein the ligand is an N-acetylgalactosamine derivative; or the delivery vehicle is a liposome, peptide, or antibody specifically targeted to liver cells.

9. The biological agent or pharmaceutical composition of claim 8, wherein the structure of the N-acetylgalactosamine derivative is as follows:

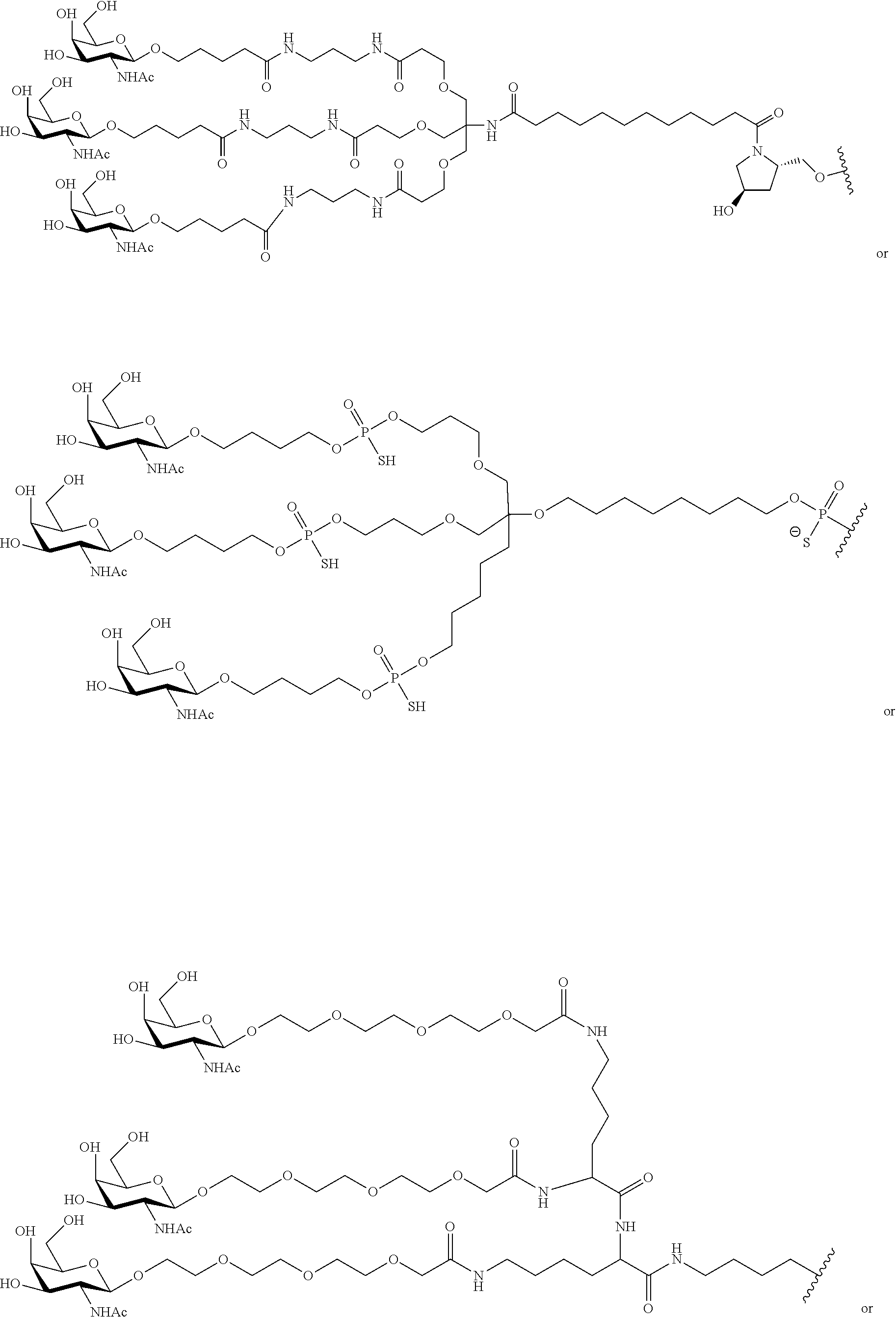

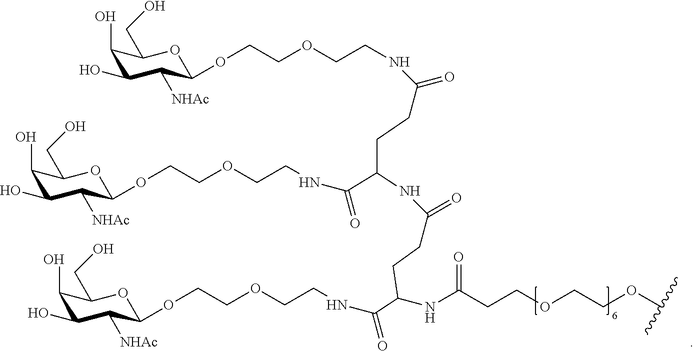

10. The biological agent or pharmaceutical composition of claim 9, wherein the N-acetylgalactosamine derivative is conjugated to the 3′-terminus of the sense strand of the siRNA, as illustrated in the following schematic:

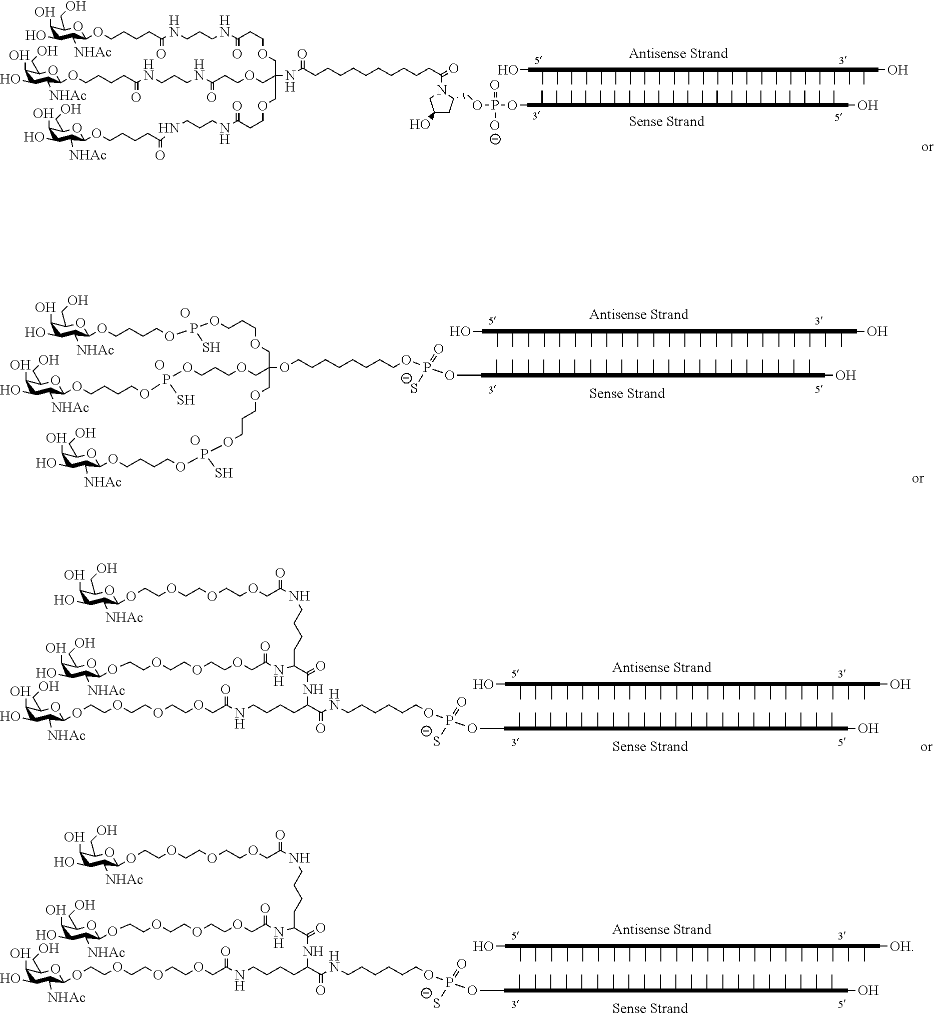

11. A method of inhibiting TMPRSS6 expression in a cell, the method comprising: (a) contacting the cell with the biological agent or pharmaceutical composition of claim 7; and (b) maintaining the cell produced in step (a) for a time sufficient to obtain
degradation of the mRNA transcript of a TMPRSS6 gene, thereby inhibiting expression of the TMPRSS6 gene in the cell.

12. A method of treating a subject suffering from a TMPRSS6-related disorder or a disease associated with iron excess or iron overload, comprising administering to the subject a therapeutically effective amount of the biological agent or pharmaceutical composition of claim 7 thereby treating the subject.

13. The method of claim 11, wherein the disorder associated with TMPRSS6 expression or diseases associated with iron excess or iron overload are selected from polycythemia, thalassemia, hemochromatosis, myelodysplastic syndrome, porphyria cutanea tarda, aplastic anemia, sideroblastic anemia, iron-refractory iron deficiency anemia, hereditary anemia, severe chronic hemolytic anemia, Parkinson's disease, Alzheimer's disease, Friedreich's ataxia, or microcytic anemia, transfusion-induced iron overload, and iron overload associated with non-alcoholic fatty liver disease (NAFLD).

14. The method of claim 13, wherein the thalassemia is selected from α-thalassemia, β-thalassemia, or 8-thalassemia.

15. The method of claim 12, wherein the polycythemia is polycythemia vera.

16. The method of claim 13, wherein the hereditary anemia is selected from sickle-cell anemia, thalassemia, Fanconi anemia, Diamond-Blackfan anemia, Shwachman-Diamond syndrome, red blood cell membrane disorders, glucose-6-phosphate dehydrogenase deficiency, and hereditary hemorrhagic telangiectasia.


SEQUENCE LISTING (188 sequences)

SEQ ID NO: 1 - RNA, 19 nt, synthetic construct
  source 1..19: mol_type = other RNA
tcacctgcttcttctggtt

SEQ ID NO: 2 - RNA, 19 nt, synthetic construct
  source 1..19: mol_type = other RNA
aaccagaagaagcaggtga

SEQ ID NO: 3 - RNA, 21 nt, synthetic construct
  source 1..21: mol_type = other RNA
gccaaagcccagaagatgcta

SEQ ID NO: 4 - RNA, 23 nt, synthetic construct
  source 1..23: mol_type = other RNA
tagcatcttctgggctttggcgg

SEQ ID NO: 5 - RNA, 21 nt, synthetic construct
  source 1..21: mol_type = other RNA
tcctttggggagggaccccta

SEQ ID NO: 6 - RNA, 23 nt, synthetic construct
  source 1..23: mol_type = other RNA
taggggtccctccccaaaggaat

SEQ ID NO: 7 - RNA, 21 nt, synthetic construct
  source 1..21: mol_type = other RNA
gtgatcctggaagccagtgta

SEQ ID NO: 8 - RNA, 23 nt, synthetic construct
  source 1..23: mol_type = other RNA
tacactggcttccaggatcacta

SEQ ID NO: 9 - RNA, 21 nt, synthetic construct
  source 1..21: mol_type = other RNA
tgatcctggaagccagtgtga

SEQ ID NO: 10 - RNA, 23 nt, synthetic construct
  source 1..23: mol_type = other RNA
tcacactggcttccaggatcacc

SEQ ID NO: 11 - RNA, 21 nt, synthetic construct
  source 1..21: mol_type = other RNA
gatcctggaagccagtgtgaa

SEQ ID NO: 12 - RNA, 23 nt, synthetic construct
  source 1..23: mol_type = other RNA
ttcacactggcttccaggatcac

SEQ ID NO: 13 - RNA, 21 nt, synthetic construct
  source 1..21: mol_type = other RNA
ccctctctggactacggctta

SEQ ID NO: 14 - RNA, 23 nt, synthetic construct
  source 1..23: mol_type = other RNA
taagccgtagtccagagagggca

SEQ ID NO: 15 - RNA, 21 nt, synthetic construct
  source 1..21: mol_type = other RNA
gatccagaacaggaggctgta

SEQ ID NO: 16 - RNA, 23 nt, synthetic construct
  source 1..23: mol_type = other RNA
tacagcctcctgttctggatcgt

SEQ ID NO: 17 - RNA, 21 nt, synthetic construct
  source 1..21: mol_type = other RNA
atccagaacaggaggctgtgt

SEQ ID NO: 18 - RNA, 23 nt, synthetic construct
  source 1..23: mol_type = other RNA
acacagcctcctgttctggatcg

SEQ ID NO: 19 - RNA, 21 nt, synthetic construct
  source 1..21: mol_type = other RNA
tccagaacaggaggctgtgta

SEQ ID NO: 20 - RNA, 23 nt, synthetic construct
  source 1..23: mol_type = other RNA
tacacagcctcctgttctggatc

SEQ ID NO: 21 - RNA, 21 nt, synthetic construct
  source 1..21: mol_type = other RNA
ccagaacaggaggctgtgtga

SEQ ID NO: 22 - RNA, 23 nt, synthetic construct
  source 1..23: mol_type = other RNA
tcacacagcctcctgttctggat

SEQ ID NO: 23 - RNA, 21 nt, synthetic construct
  source 1..21: mol_type = other RNA
cagaacaggaggctgtgtgga

SEQ ID NO: 24 - RNA, 23 nt, synthetic construct
  source 1..23: mol_type = other RNA
tccacacagcctcctgttctgga

SEQ ID NO: 25 - RNA, 21 nt, synthetic construct
  source 1..21: mol_type = other RNA
gaacaggaggctgtgtggctt

SEQ ID NO: 26 - RNA, 23 nt, synthetic construct
  source 1..23: mol_type = other RNA
aagccacacagcctcctgttctg

SEQ ID NO: 27 - RNA, 21 nt, synthetic construct
  source 1..21: mol_type = other RNA
tcaccatcaacttcacctcca

SEQ ID NO: 28 - RNA, 23 nt, synthetic construct
  source 1..23: mol_type = other RNA
tggaggtgaagttgatggtgatc

SEQ ID NO: 29 - RNA, 21 nt, synthetic construct
  source 1..21: mol_type = other RNA
caccatcaacttcacctccca

SEQ ID NO: 30 - RNA, 23 nt, synthetic construct
  source 1..23: mol_type = other RNA
tgggaggtgaagttgatggtgat

SEQ ID NO: 31 - RNA, 21 nt, synthetic construct
  source 1..21: mol_type = other RNA
accatcaacttcacctcccaa

SEQ ID NO: 32 - RNA, 23 nt, synthetic construct
  source 1..23: mol_type = other RNA
ttgggaggtgaagttgatggtga

SEQ ID NO: 33 - RNA, 21 nt, synthetic construct
  source 1..21: mol_type = other RNA
ccatcaacttcacctcccaga

SEQ ID NO: 34 - RNA, 23 nt, synthetic construct
  source 1..23: mol_type = other RNA
tctgggaggtgaagttgatggtg

SEQ ID NO: 35 - RNA, 21 nt, synthetic construct
  source 1..21: mol_type = other RNA
catcaacttcacctcccagat

SEQ ID NO: 36 - RNA, 23 nt, synthetic construct
  source 1..23: mol_type = other RNA
atctgggaggtgaagttgatggt

SEQ ID NO: 37 - RNA, 21 nt, synthetic construct
  source 1..21: mol_type = other RNA
atcaacttcacctcccagata

SEQ ID NO: 38 - RNA, 23 nt, synthetic construct
  source 1..23: mol_type = other RNA
tatctgggaggtgaagttgatgg

SEQ ID NO: 39 - RNA, 21 nt, synthetic construct
  source 1..21: mol_type = other RNA
tcaacttcacctcccagatct

SEQ ID NO: 40 - RNA, 23 nt, synthetic construct
  source 1..23: mol_type = other RNA
agatctgggaggtgaagttgatg

SEQ ID NO: 41 - RNA, 21 nt, synthetic construct
  source 1..21: mol_type = other RNA
caacttcacctcccagatcta

SEQ ID NO: 42 - RNA, 23 nt, synthetic construct
  source 1..23: mol_type = other RNA
tagatctgggaggtgaagttgat

SEQ ID NO: 43 - RNA, 21 nt, synthetic construct
  source 1..21: mol_type = other RNA
aacttcacctcccagatctca

SEQ ID NO: 44 - RNA, 23 nt, synthetic construct
  source 1..23: mol_type = other RNA
tgagatctgggaggtgaagttga

SEQ ID NO: 45 - RNA, 21 nt, synthetic construct
  source 1..21: mol_type = other RNA
acttcacctcccagatctcca

SEQ ID NO: 46 - RNA, 23 nt, synthetic construct
  source 1..23: mol_type = other RNA
tggagatctgggaggtgaagttg

SEQ ID NO: 47 - RNA, 21 nt, synthetic construct
  source 1..21: mol_type = other RNA
cttcacctcccagatctccct

SEQ ID NO: 48 - RNA, 23 nt, synthetic construct
  source 1..23: mol_type = other RNA
agggagatctgggaggtgaagtt

SEQ ID NO: 49 - RNA, 21 nt, synthetic construct
  source 1..21: mol_type = other RNA
ttcacctcccagatctcccta

SEQ ID NO: 50 - RNA, 23 nt, synthetic construct
  source 1..23: mol_type = other RNA
tagggagatctgggaggtgaagt

SEQ ID NO: 51 - RNA, 21 nt, synthetic construct
  source 1..21: mol_type = other RNA
tcacctcccagatctccctca

SEQ ID NO: 52 - RNA, 23 nt, synthetic construct
  source 1..23: mol_type = other RNA
tgagggagatctgggaggtgaag

SEQ ID NO: 53 - RNA, 21 nt, synthetic construct
  source 1..21: mol_type = other RNA
cacctcccagatctccctcaa

SEQ ID NO: 54 - RNA, 23 nt, synthetic construct
  source 1..23: mol_type = other RNA
ttgagggagatctgggaggtgaa

SEQ ID NO: 55 - RNA, 21 nt, synthetic construct
  source 1..21: mol_type = other RNA
ccagtgtgaggaccggagcta

SEQ ID NO: 56 - RNA, 23 nt, synthetic construct
  source 1..23: mol_type = other RNA
tagctccggtcctcacactggaa

SEQ ID NO: 57 - RNA, 21 nt, synthetic construct
  source 1..21: mol_type = other RNA
cactgtgactgtggcctccaa

SEQ ID NO: 58 - RNA, 23 nt, synthetic construct
  source 1..23: mol_type = other RNA
ttggaggccacagtcacagtgct

SEQ ID NO: 59 - RNA, 21 nt, synthetic construct
  source 1..21: mol_type = other RNA
ttcggggtcgacacatctgta

SEQ ID NO: 60 - RNA, 23 nt, synthetic construct
  source 1..23: mol_type = other RNA
tacagatgtgtcgaccccgaacc

SEQ ID NO: 61 - RNA, 21 nt, synthetic construct
  source 1..21: mol_type = other RNA
tcggggtcgacacatctgtga

SEQ ID NO: 62 - RNA, 23 nt, synthetic construct
  source 1..23: mol_type = other RNA
tcacagatgtgtcgaccccgaac

SEQ ID NO: 63 - RNA, 21 nt, synthetic construct
  source 1..21: mol_type = other RNA
cggggtcgacacatctgtgga

SEQ ID NO: 64 - RNA, 23 nt, synthetic construct
  source 1..23: mol_type = other RNA
tccacagatgtgtcgaccccgaa

SEQ ID NO: 65 - RNA, 21 nt, synthetic construct
  source 1..21: mol_type = other RNA
ccaggaggacagcatggccta

SEQ ID NO: 66 - RNA, 23 nt, synthetic construct
  source 1..23: mol_type = other RNA
taggccatgctgtcctcctggaa

SEQ ID NO: 67 - RNA, 21 nt, synthetic construct
  source 1..21: mol_type = other RNA
cttccaggaggacagcatgga

SEQ ID NO: 68 - RNA, 23 nt, synthetic construct
  source 1..23: mol_type = other RNA
tccatgctgtcctcctggaagca

SEQ ID NO: 69 - RNA, 21 nt, synthetic construct
  source 1..21: mol_type = other RNA
gcttccaggaggacagcatga

SEQ ID NO: 70 - RNA, 23 nt, synthetic construct
  source 1..23: mol_type = other RNA
tcatgctgtcctcctggaagcag

SEQ ID NO: 71 - RNA, 21 nt, synthetic construct
  source 1..21: mol_type = other RNA
tgcttccaggaggacagcata

SEQ ID NO: 72 - RNA, 23 nt, synthetic construct
  source 1..23: mol_type = other RNA
tatgctgtcctcctggaagcagt

SEQ ID NO: 73 - RNA, 21 nt, synthetic construct
  source 1..21: mol_type = other RNA
ctgcttccaggaggacagcat

SEQ ID NO: 74 - RNA, 23 nt, synthetic construct
  source 1..23: mol_type = other RNA
atgctgtcctcctggaagcagtg

SEQ ID NO: 75 - RNA, 21 nt, synthetic construct
  source 1..21: mol_type = other RNA
gtgtccttcaaggtgagccga

SEQ ID NO: 76 - RNA, 23 nt, synthetic construct
  source 1..23: mol_type = other RNA
tcggctcaccttgaaggacacct

SEQ ID NO: 77 - RNA, 21 nt, synthetic construct
  source 1..21: mol_type = other RNA
ggtgtccttcaaggtgagcca

SEQ ID NO: 78 - RNA, 23 nt, synthetic construct
  source 1..23: mol_type = other RNA
tggctcaccttgaaggacacctc

SEQ ID NO: 79 - RNA, 21 nt, synthetic construct
  source 1..21: mol_type = other RNA
aggtgtccttcaaggtgagca

SEQ ID NO: 80 - RNA, 23 nt, synthetic construct
  source 1..23: mol_type = other RNA
tgctcaccttgaaggacacctct

SEQ ID NO: 81 - RNA, 21 nt, synthetic construct
  source 1..21: mol_type = other RNA
agccatgactacgacgtggca

SEQ ID NO: 82 - RNA, 23 nt, synthetic construct
  source 1..23: mol_type = other RNA
tgccacgtcgtagtcatggctgt

SEQ ID NO: 83 - RNA, 21 nt, synthetic construct
  source 1..21: mol_type = other RNA
cagccatgactacgacgtgga

SEQ ID NO: 84 - RNA, 23 nt, synthetic construct
  source 1..23: mol_type = other RNA
tccacgtcgtagtcatggctgtc

SEQ ID NO: 85 - RNA, 21 nt, synthetic construct
  source 1..21: mol_type = other RNA
acagccatgactacgacgtga

SEQ ID NO: 86 - RNA, 23 nt, synthetic construct
  source 1..23: mol_type = other RNA
tcacgtcgtagtcatggctgtcc

SEQ ID NO: 87 - RNA, 21 nt, synthetic construct
  source 1..21: mol_type = other RNA
gacagccatgactacgacgta

SEQ ID NO: 88 - RNA, 23 nt, synthetic construct
  source 1..23: mol_type = other RNA
tacgtcgtagtcatggctgtcct

SEQ ID NO: 89 - RNA, 21 nt, synthetic construct
  source 1..21: mol_type = other RNA
ggacagccatgactacgacgt

SEQ ID NO: 90 - RNA, 23 nt, synthetic construct
  source 1..23: mol_type = other RNA
acgtcgtagtcatggctgtcctc

SEQ ID NO: 91 - RNA, 21 nt, synthetic construct
  source 1..21: mol_type = other RNA
aggacagccatgactacgaca

SEQ ID NO: 92 - RNA, 23 nt, synthetic construct
  source 1..23: mol_type = other RNA
tgtcgtagtcatggctgtcctct

SEQ ID NO: 93 - RNA, 21 nt, synthetic construct
  source 1..21: mol_type = other RNA
gaggacagccatgactacgaa

SEQ ID NO: 94 - RNA, 23 nt, synthetic construct
  source 1..23: mol_type = other RNA
ttcgtagtcatggctgtcctctt

SEQ ID NO: 95 - RNA, 21 nt, synthetic construct
  source 1..21: mol_type = other RNA
cgcctgggaacttactacaat

SEQ ID NO: 96 - RNA, 23 nt, synthetic construct
  source 1..23: mol_type = other RNA
attgtagtaagttcccaggcggg

SEQ ID NO: 97 - RNA, 21 nt, synthetic construct
  source 1..21: mol_type = other RNA
gcctgggaacttactacaact

SEQ ID NO: 98 - RNA, 23 nt, synthetic construct
  source 1..23: mol_type = other RNA
agttgtagtaagttcccaggcgg

SEQ ID NO: 99 - RNA, 21 nt, synthetic construct
  source 1..21: mol_type = other RNA
cctgggaacttactacaacta

SEQ ID NO: 100 - RNA, 23 nt, synthetic construct
  source 1..23: mol_type = other RNA
tagttgtagtaagttcccaggcg

SEQ ID NO: 101 - RNA, 21 nt, synthetic construct
  source 1..21: mol_type = other RNA
ctgggaacttactacaactca

SEQ ID NO: 102 - RNA, 23 nt, synthetic construct
  source 1..23: mol_type = other RNA
tgagttgtagtaagttcccaggc

SEQ ID NO: 103 - RNA, 21 nt, synthetic construct
  source 1..21: mol_type = other RNA
tgggaacttactacaactcca

SEQ ID NO: 104 - RNA, 23 nt, synthetic construct
  source 1..23: mol_type = other RNA
tggagttgtagtaagttcccagg

SEQ ID NO: 105 - RNA, 21 nt, synthetic construct
  source 1..21: mol_type = other RNA
gggaacttactacaactccaa

SEQ ID NO: 106 - RNA, 23 nt, synthetic construct
  source 1..23: mol_type = other RNA
ttggagttgtagtaagttcccag

SEQ ID NO: 107 - RNA, 21 nt, synthetic construct
  source 1..21: mol_type = other RNA
ggaacttactacaactccaga

SEQ ID NO: 108 - RNA, 23 nt, synthetic construct
  source 1..23: mol_type = other RNA
tctggagttgtagtaagttccca

SEQ ID NO: 109 - RNA, 21 nt, synthetic construct
  source 1..21: mol_type = other RNA
gaacttactacaactccagct

SEQ ID NO: 110 - RNA, 23 nt, synthetic construct
  source 1..23: mol_type = other RNA
agctggagttgtagtaagttccc

SEQ ID NO: 111 - RNA, 21 nt, synthetic construct
  source 1..21: mol_type = other RNA
aacttactacaactccagcta

SEQ ID NO: 112 - RNA, 23 nt, synthetic construct
  source 1..23: mol_type = other RNA
tagctggagttgtagtaagttcc

SEQ ID NO: 113 - RNA, 21 nt, synthetic construct
  source 1..21: mol_type = other RNA
acttactacaactccagctca

SEQ ID NO: 114 - RNA, 23 nt, synthetic construct
  source 1..23: mol_type = other RNA
tgagctggagttgtagtaagttc

SEQ ID NO: 115 - RNA, 21 nt, synthetic construct
  source 1..21: mol_type = other RNA
cttactacaactccagctcca

SEQ ID NO: 116 - RNA, 23 nt, synthetic construct
  source 1..23: mol_type = other RNA
tggagctggagttgtagtaagtt

SEQ ID NO: 117 - RNA, 21 nt, synthetic construct
  source 1..21: mol_type = other RNA
ttactacaactccagctccgt

SEQ ID NO: 118 - RNA, 23 nt, synthetic construct
  source 1..23: mol_type = other RNA
acggagctggagttgtagtaagt

SEQ ID NO: 119 - RNA, 21 nt, synthetic construct
  source 1..21: mol_type = other RNA
tactacaactccagctccgta

SEQ ID NO: 120 - RNA, 23 nt, synthetic construct
  source 1..23: mol_type = other RNA
tacggagctggagttgtagtaag

SEQ ID NO: 121 - RNA, 21 nt, synthetic construct
  source 1..21: mol_type = other RNA
actacaactccagctccgtct

SEQ ID NO: 122 - RNA, 23 nt, synthetic construct
  source 1..23: mol_type = other RNA
agacggagctggagttgtagtaa

SEQ ID NO: 123 - RNA, 21 nt, synthetic construct
  source 1..21: mol_type = other RNA
ctacaactccagctccgtcta

SEQ ID NO: 124 - RNA, 23 nt, synthetic construct
  source 1..23: mol_type = other RNA
tagacggagctggagttgtagta

SEQ ID NO: 125 - RNA, 21 nt, synthetic construct
  source 1..21: mol_type = other RNA
tacaactccagctccgtctat

SEQ ID NO: 126 - RNA, 23 nt, synthetic construct
  source 1..23: mol_type = other RNA
atagacggagctggagttgtagt

SEQ ID NO: 127 - RNA, 21 nt, synthetic construct
  source 1..21: mol_type = other RNA
acaactccagctccgtctatt

SEQ ID NO: 128 - RNA, 23 nt, synthetic construct
  source 1..23: mol_type = other RNA
aatagacggagctggagttgtag

SEQ ID NO: 129 - RNA, 21 nt, synthetic construct
  source 1..21: mol_type = other RNA
caactccagctccgtctatta

SEQ ID NO: 130 - RNA, 23 nt, synthetic construct
  source 1..23: mol_type = other RNA
taatagacggagctggagttgta

SEQ ID NO: 131 - RNA, 21 nt, synthetic construct
  source 1..21: mol_type = other RNA
aactccagctccgtctattca

SEQ ID NO: 132 - RNA, 23 nt, synthetic construct
  source 1..23: mol_type = other RNA
tgaatagacggagctggagttgt

SEQ ID NO: 133 - RNA, 21 nt, synthetic construct
  source 1..21: mol_type = other RNA
actccagctccgtctattcct

SEQ ID NO: 134 - RNA, 23 nt, synthetic construct
  source 1..23: mol_type = other RNA
aggaatagacggagctggagttg

SEQ ID NO: 135 - RNA, 21 nt, synthetic construct
  source 1..21: mol_type = other RNA
ctccagctccgtctattcctt

SEQ ID NO: 136 - RNA, 23 nt, synthetic construct
  source 1..23: mol_type = other RNA
aaggaatagacggagctggagtt

SEQ ID NO: 137 - RNA, 21 nt, synthetic construct
  source 1..21: mol_type = other RNA
tccaggcctgtgaagtgaaca

SEQ ID NO: 138 - RNA, 23 nt, synthetic construct
  source 1..23: mol_type = other RNA
tgttcacttcacaggcctggaag

SEQ ID NO: 139 - RNA, 21 nt, synthetic construct
  source 1..21: mol_type = other RNA
ccaggcctgtgaagtgaacct

SEQ ID NO: 140 - RNA, 23 nt, synthetic construct
  source 1..23: mol_type = other RNA
aggttcacttcacaggcctggaa

SEQ ID NO: 141 - RNA, 21 nt, synthetic construct
  source 1..21: mol_type = other RNA
caggcctgtgaagtgaaccta

SEQ ID NO: 142 - RNA, 23 nt, synthetic construct
  source 1..23: mol_type = other RNA
taggttcacttcacaggcctgga

SEQ ID NO: 143 - RNA, 21 nt, synthetic construct
  source 1..21: mol_type = other RNA
aggcctgtgaagtgaacctga

SEQ ID NO: 144 - RNA, 23 nt, synthetic construct
  source 1..23: mol_type = other RNA
tcaggttcacttcacaggcctgg

SEQ ID NO: 145 - RNA, 21 nt, synthetic construct
  source 1..21: mol_type = other RNA
ggcctgtgaagtgaacctgaa

SEQ ID NO: 146 - RNA, 23 nt, synthetic construct
  source 1..23: mol_type = other RNA
ttcaggttcacttcacaggcctg

SEQ ID NO: 147 - RNA, 21 nt, synthetic construct
  source 1..21: mol_type = other RNA
gcctgtgaagtgaacctgaca

SEQ ID NO: 148 - RNA, 23 nt, synthetic construct
  source 1..23: mol_type = other RNA
tgtcaggttcacttcacaggcct

SEQ ID NO: 149 - RNA, 21 nt, synthetic construct
  source 1..21: mol_type = other RNA
ctgtgaagtgaacctgacgct

SEQ ID NO: 150 - RNA, 23 nt, synthetic construct
  source 1..23: mol_type = other RNA
agcgtcaggttcacttcacaggc

SEQ ID NO: 151 - RNA, 21 nt, synthetic construct
  source 1..21: mol_type = other RNA
tgtgaagtgaacctgacgcta

SEQ ID NO: 152 - RNA, 23 nt, synthetic construct
  source 1..23: mol_type = other RNA
tagcgtcaggttcacttcacagg

SEQ ID NO: 153 - RNA, 21 nt, synthetic construct
  source 1..21: mol_type = other RNA
gtgaagtgaacctgacgctga

SEQ ID NO: 154 - RNA, 23 nt, synthetic construct
  source 1..23: mol_type = other RNA
tcagcgtcaggttcacttcacag

SEQ ID NO: 155 - RNA, 21 nt, synthetic construct
  source 1..21: mol_type = other RNA
atcgctgaccgctgggtgata

SEQ ID NO: 156 - RNA, 23 nt, synthetic construct
  source 1..23: mol_type = other RNA
tatcacccagcggtcagcgatga

SEQ ID NO: 157 - RNA, 21 nt, synthetic construct
  source 1..21: mol_type = other RNA
tcgctgaccgctgggtgataa

SEQ ID NO: 158 - RNA, 23 nt, synthetic construct
  source 1..23: mol_type = other RNA
ttatcacccagcggtcagcgatg

SEQ ID NO: 159 - RNA, 21 nt, synthetic construct
  source 1..21: mol_type = other RNA
cgctgaccgctgggtgataat

SEQ ID NO: 160 - RNA, 23 nt, synthetic construct
  source 1..23: mol_type = other RNA
attatcacccagcggtcagcgat

SEQ ID NO: 161 - RNA, 21 nt, synthetic construct
  source 1..21: mol_type = other RNA
gctgaccgctgggtgataaca

SEQ ID NO: 162 - RNA, 23 nt, synthetic construct
  source 1..23: mol_type = other RNA
tgttatcacccagcggtcagcga

SEQ ID NO: 163 - RNA, 21 nt, synthetic construct
  source 1..21: mol_type = other RNA
gaccgctgggtgataacagct

SEQ ID NO: 164 - RNA, 23 nt, synthetic construct
  source 1..23: mol_type = other RNA
agctgttatcacccagcggtcag

SEQ ID NO: 165 - RNA, 21 nt, synthetic construct
  source 1..21: mol_type = other RNA
accgctgggtgataacagcta

SEQ ID NO: 166 - RNA, 23 nt, synthetic construct
  source 1..23: mol_type = other RNA
tagctgttatcacccagcggtca

SEQ ID NO: 167 - RNA, 21 nt, synthetic construct
  source 1..21: mol_type = other RNA
tgctactctggtatttcctaa

SEQ ID NO: 168 - RNA, 23 nt, synthetic construct
  source 1..23: mol_type = other RNA
ttaggaaataccagagtagcacc

SEQ ID NO: 169 - RNA, 21 nt, synthetic construct
  source 1..21: mol_type = other RNA
gtgctactctggtatttccta

SEQ ID NO: 170 - RNA, 23 nt, synthetic construct
  source 1..23: mol_type = other RNA
taggaaataccagagtagcaccc

SEQ ID NO: 171 - RNA, 21 nt, synthetic construct
  source 1..21: mol_type = other RNA
ggtgctactctggtatttcct

SEQ ID NO: 172 - RNA, 23 nt, synthetic construct
  source 1..23: mol_type = other RNA
aggaaataccagagtagcacccc

SEQ ID NO: 173 - RNA, 21 nt, synthetic construct
  source 1..21: mol_type = other RNA
gggtgctactctggtatttca

SEQ ID NO: 174 - RNA, 23 nt, synthetic construct
  source 1..23: mol_type = other RNA
tgaaataccagagtagcaccccc

SEQ ID NO: 175 - RNA, 21 nt, synthetic construct
  source 1..21: mol_type = other RNA
ggggtgctactctggtattta

SEQ ID NO: 176 - RNA, 23 nt, synthetic construct
  source 1..23: mol_type = other RNA
taaataccagagtagcacccccg

SEQ ID NO: 177 - RNA, 21 nt, synthetic construct
  source 1..21: mol_type = other RNA
gggggtgctactctggtattt

SEQ ID NO: 178 - RNA, 23 nt, synthetic construct
  source 1..23: mol_type = other RNA
aaataccagagtagcacccccgc

SEQ ID NO: 179 - RNA, 21 nt, synthetic construct
  source 1..21: mol_type = other RNA
cgggggtgctactctggtatt

SEQ ID NO: 180 - RNA, 23 nt, synthetic construct
  source 1..23: mol_type = other RNA
aataccagagtagcacccccgcc

SEQ ID NO: 181 - RNA, 21 nt, synthetic construct
  source 1..21: mol_type = other RNA
gctactctggtatttcctagg

SEQ ID NO: 182 - RNA, 23 nt, synthetic construct
  source 1..23: mol_type = other RNA
cctaggaaataccagagtagcac

SEQ ID NO: 183 - RNA, 21 nt, synthetic construct
  source 1..21: mol_type = other RNA
ctactctggtatttcctaggg

SEQ ID NO: 184 - RNA, 23 nt, synthetic construct
  source 1..23: mol_type = other RNA
ccctaggaaataccagagtagca

SEQ ID NO: 185 - RNA, 21 nt, synthetic construct
  source 1..21: mol_type = other RNA
tactctggtatttcctagggt

SEQ ID NO: 186 - RNA, 23 nt, synthetic construct
  source 1..23: mol_type = other RNA
accctaggaaataccagagtagc

SEQ ID NO: 187 - RNA, 21 nt, synthetic construct
  source 1..21: mol_type = other RNA
tgctactctggtatttcctat

SEQ ID NO: 188 - RNA, 23 nt, synthetic construct
  source 1..23: mol_type = other RNA
ataggaaataccagagtagcacc